SEQUENCE LISTING (58 sequences)

SEQ ID NO: 1 - protein, 4 aa, Artificial sequence
  Synthetic construct
KDEL

SEQ ID NO: 2 - protein, 4 aa, Artificial sequence
  Synthetic construct
HDEL

SEQ ID NO: 3 - DNA, 2629 bp, Unknown
  Pestivirus genus of the Flaviviridae
atgaaagccctattggcatgggcagtgatagcaatcgcgctgtaccagcctgtagcagccgagaatataactcaatggaacctgagtgacaacggcacaagcggcatccagcgagctatgtatctcagaggggtcaataggagcttacatgggatctggcctgaaaaaatatgcaagggggtccccactcatctggccactgatacggaactgacagagatacgcgggatgatggacgccagtgagaggacgaactatacgtgttgtaggttgcagagacacgaatggaataaacatggatggtgtaactggtacaacatagacccttggattcagttaatgaacaggacccaagcaaatttgacagaaggccctccaaataaagagtgcgctgtgacttgcaggtatgacaacaataccgacgttaacgtggttacccaggccaggaatagaccaaccactctgactggctgcaagaaagggaaaaatttttcatttgcaggtacggtcatagagggcccatgcaatttcaacgtatccgtggaggacatcttatatggagaccatgagtgtggcagcctgtttcaggacacggctctgtacctattagatggaatgaccaacactatagagaaagccaggcagggtgcggcaagagtcacatcttggctagggaggcaactcagtaccacagggaagaagttggagagaggaagcaaaacctggttcggtgcctatgccctgtcaccttattgcaatgtaacaaggaaaatagggtacatatggtacacgaacaattgcactccggcatgcctcccaaaaaacacaaaaataataggcccaggaaaatttgataccaacgcagaagacgggaagattcttcatgaaatgggggccacctatcagagttcttgttcgtttctctggtaatcctgtctgacttcgccccagaaacagccagtacgttatacctaatcttacactatgcaatccctcagtcctatgaagaacctgagggttgcgatatgaaccagctcaacctaacggtgggacttaggacagaagatgtggtaccatcatcagtctggaatattggtaaatatgtgtgtgttagaccagattggtggccgtatgaaactaaggtggctctactgtttgaagaggtaggacaggttataaagctagctctacgggcactgagggatttaactagagtctggaacagtgcatcaaccactgcattcctcatctgcttgataaaaatattaagaggacaggttgtgcaaggtataatatggctgctgctggtgaccggggcacaagggcggttgtcctgcaaggaagactacaggtatgcgatatcatcaaccaatgagatagggccgctaggggctgaaggtctcactaccacctggaaagaatacaaccacggtttgcagctggatgacgggacagtcagggccatctgcactgcagggtcctttaaagttatagcacttaatgtggtcagtaggagatacctggcatcattacacaagagggctttgcccacctcagtaacatttgaactcctatttgatgggactagtccagcaatagaggagatgggagatgactttggatttgggctgtgcccttttgacacaacccccgtggtaaaagggaagtacaataccactttgttaaacggtagtgctttctacctagtctgcccaataggatggacgggtgtcatagagtgcacggcagtaagcctcacaaccttgagaacagaagtggtgaagaccttcaagagagagaagcctttcccgcacagagtggattgcgtgaccactatagtagaaaaagaagacctgttctactgcaagttgggggtaattggacatgtgtgaaaggcaacccggtgacctacatgggggggcaagtaaaacaatgcaggtggtgcggttttgacttcaaggagcccgatgggctcccacactaccccataggcaagtgcatcctagcaaatgagacgggttacagggtagtggattccacagactgcaacagagatggcgtcgttatcagcactgaaggagaacacgagtgcttgattggcaacaccaccgtcaaggtacacgcgttggatggaagactgggccccatgccgtgcagacccaaagaaattgtctctagtgcgggacctgtaaggaaaacttcctgcactttcaattacacaaagacactaagaaataagtactatgagcccaaggacagctatttccagcaatatatgcttaagggcgagtaccaatactggtttgatctggacgtgactgaccaccacacagactactttgccgaatttgttgtcttggtggtagtggcactattaggggggaggtacgttctgtggctaatagtgacctatatagttctaacagagcaactcgctgctggtttacagctaggccaaggcgaggcggtactgatagggaacttaatcacccacacggacaatgaggtggtggtatacttcttactgctctacctgataataagagacgagcccataaagaaatggatactactgctgtttcatggaattctgcagatatccagcacagtggcggccgctcgagtctag

SEQ ID NO: 4 - DNA, 1308 bp, Unknown
  Pestivirus genus of the Flaviviridae
atgcggctagcctgcaaggaagattacaggtatgcgatatcatcaaccaatgagatagggccgctaggggctgaaggtctcactaccacctggaaagaatacaaccatggtttgcagctggatgacgggacagtcagggccatctgcactgcagggtcctttaaagttatagcacttaatgtggtcagtaggagatacctggcatcattacacaagagggctttgcccacctcagtaacatttgaactcctatttgatgggactagtccagcaatagaggagatgggagatgactttggatttgggctgtgcccttttgacacaacccccgtggtaaaagggaagtacaataccactttgttaaacggtagtgctttctacctagtctgcccaataggatggacgggtgtcatagagtgcacggcagtaagcctcacaaccttgagaacagaagtggtgaagaccttcaagagagagaagcctttcccgcacagagtggattgcgtgaccactatagtagaaaaagaagacctgttctactgcaagttggggggtaattggacatgtgtgaaaggcaacccggtgacctacatgggggggcaagtaaaacaatgcaggtggtgcggttttgacttcaaggagcccgatgggctcccacactaccccataggcaagtgcatcctagcaaatgagacgggttacagggtagtggattccacagactgcaacagagatggcgtcgttatcagcactgaaggagaacacgagtgcttgattggcaacaccaccgtcaaggtacacgcgttggatggaagactgggccccatgccgtgcagacccaaagaaattgtctctagtgcgggacctgtaaggaaaacttcctgcactttcaattacacaaagacactaagaaataagtactatgagcccaaggacagctatttccagcaatatatgcttaagggcgagtaccaatactgatttgatctggacgtgactgaccaccacacagactactttgccgaatttgttgtcttggtggtagtggcactattaggggggaggtacgttctgtggctaatagtgacctatatagttctaacagagcaactcgctgctggtttacagctaggccaaggcgaggcggtactgatagggaacttaatcacccacacggacaatgaggtggtggtatacttcttactgctctacctgataataagagacgagcccataaagaaatggatactactgctgtttcatggaattctgcagatatccagcacagtggcggccgctcgagtctag

SEQ ID NO: 5 - DNA, 1266 bp, Unknown
  Pestivirus genus of the Flaviviridae
atgcggctagcctgcaaggaagattacaggtatgcgatatcatcaaccaatgagatagggccgctaggggctgaaggtctcactaccacctggaaagaatacaaccatggtttgcagctggatgacgggacagtcaaggccatctgcactgcagggtcctttaaagttatagcacttaatgtggtcagtaggagatacctggcatcattacacaagagggctttgcccacctcagtaacatttgaactcctatttgatgggactagtccagcaatagaggagatgggagatgactttggatttgggctgtgcccttttgacacaacccccgtggtaaaagggaagtacaataccactttgttaaacggtagtgctttctacctagtctgcccaataggatggacgggtgtcatagagtgcacggcagtaagcctcacaaccttgagaacagaagtggtgaagaccttcaagagagagaagcctttcccgcacagagtggattgcgtgaccactatagtagaaaaagaagacctgttctactgcaagttggggggtaattggacatgtgtgaaaggcaacccggtgacctacatgggggggcaagtaaaacaatgcaggtggtgcggttttgacttcaaggagcccgatgggctcccacactaccccataggcaagtgcatcctagcaaatgagacgggttacagggtagtggattccacagactgcaacagagatggcgtcgttatcagcactgaaggagaacacgagtgcttgattggcaacaccaccgtcaaggtacacgcgttggatggaagactgggccccatgccgtgcagacccaaagaaattgtctctagtgcgggacctgtaaggaaaacttcctgcactttcaattacacaaagacactaagaaataagtactatgagcccaaggacagctatttccagcaatatatgcttaagggcgagtaccaatactggtttgatctggacgtgactgaccaccacacagactactttgccgaatttgttgtcttggtggtagtggcactattaggggggaggtacgttctgtggctaatagtgacctatatagttctaacagagcaactcgctgctggtttacagctaggccaaggcgaggcggtactgatagggaacttaatcacccacacggacaatgaggtggtggtatacttcttactgctctacctgataataagagacgagcccataaagaaatggatactactgctgtttcattag

SEQ ID NO: 6 - DNA, 1266 bp, Unknown
  Pestivirus genus of the Flaviviridae
atgcggcttgcctgtaaggaagattacaggtatgcgatatcatcaaccaatgagatagggccgctaggggctgaaggtctcactaccacctggaaagaatacaaccatggtttgcagctggatgacgggacagtcagggccatctgcactgcagggtcctttaaagttatagcacttaatgtggtcagtaggagatacctggcatcattacacaagagggctttgcccacctcagtaacatttgaactcctatttgatgggactagtccagcaatagaggagatgggagatgactttggatttggactttgtccttttgacacaaccccagtggttaaaggtaagtacaataccactttgttaaacggtagtgctttctacctagtctgcccaataggatggacgggtgtcatagagtgcacggcagtaagcctcacaaccttgagaacagaagtggtgaagaccttcaagagagagaagcctttcccgcacagagtggattgcgtgaccactatagtagaaaaagaagacctgttctactgcaagttggggggtaattggacatgtgtgaaaggcaacccggtgacctacatgggtggacaagttaaacaatgtaggtggtgtggttttgacttcaaggagcccgattggctctcacactaccctattggtaagtgcatcctagcaaatgagacgggttacagggtagtggattccacagactgcaacagagatggcgtcgttatcagcactgaaggagaacacgagtgcttgattggcaacaccaccgtcaaggtacacgcgttggatggaagacttggtccaatgccgtgtagaccgaaagaaattgtctctagtgcgggacctgtaaggaaaacttcctgcactttcaattacacaaagacactaagaaataagtactatgagcccaaggacagctatttccagcaatatatgcttaagggcgagtaccaatactggtttgatctggacgtgactgaccaccacacagactactttgccgaatttgttgtcttggtggttgtggcacttttaggaggtaggtacgttctttggctaatagtgacctatatagttctaacagagcaactcgctgctggtttacagctaggccaaggcgaggcggtactgatagggaacttaatcacccacacggacaatgaggtggtggtatacttcttactgctctacctgataataagagacgagcccataaagaaatggattcttcttctctttcattag

SEQ ID NO: 7 - DNA, 20 bp, Artificial Sequence
  Synthetic oligonucleotide derived from Arabidopsis thaliana
atcttccatgtcgcaactcc

SEQ ID NO: 8 - DNA, 20 bp, Artificial Sequence
  Synthetic oligonucleotide derived from Arabidopsis thaliana
caaatgtagcgaccagaagc

SEQ ID NO: 9 - DNA, 28 bp, Unknown
  Pestivirus genus of the Falviviridae
gtaggatccatgcggcttgcctgtaagg

SEQ ID NO: 10 - DNA, 40 bp, Unknown
  Pestivirus genus of the Falviviridae
aaaaccacaccacctacattgtttaacttgtccacccatg

SEQ ID NO: 11 - DNA, 39 bp, Unknown
  Pestivirus genus of the Falviviridae
ataggatccatgaaagccctattggcatgggcagtgatg

SEQ ID NO: 12 - DNA, 39 bp, Unknown
  Pestivirus genus of the Falviviridae
cccgggaattccatgaaacagcagtagtatccatttctt

SEQ ID NO: 13 - DNA, 36 bp, Unknown
  Pestivirus genus of the Falviviridae
attggatccatgcggctagcctgcaaggaagattac

SEQ ID NO: 14 - DNA, 22 bp, Unknown
  Pestivirus genus of the Falviviridae
gacagcatcattacacaagagg

SEQ ID NO: 15 - DNA, 48 bp, Unknown
  Pestivirus genus of the Falviviridae
gtacttacctttaaccactggggttgtgtcaaaaggacaaagtccaaa

SEQ ID NO: 16 - DNA, 33 bp, Unknown
  Pestivirus genus of the Falviviridae
tctttcggtctacacggcattggaccaagtctt

SEQ ID NO: 17 - DNA, 18 bp, Unknown
  Genus Anterivirus of Arteriviridae
gtatgaacttgcaggatg

SEQ ID NO: 18 - DNA, 20 bp, Unknown
  Genus Anterivirus of Arteriviridae
gccgacaataccatgtgctg

SEQ ID NO: 19 - DNA, 18 bp, Unknown
  Genus Anterivirus of Arteriviridae
ggcgcagtgactaagaga

SEQ ID NO: 20 - DNA, 21 bp, Unknown
  Genus Anterivirus of Arteriviridae
gtaactgaacaccatatgctg

SEQ ID NO: 21 - DNA, 18 bp, Unknown
  Genus Anterivirus of Arteriviridae
cctcctgtatgaacttgc

SEQ ID NO: 22 - DNA, 20 bp, Unknown
  Genus Anterivirus of Arteriviridae
aggtcctcgaacttgagctg

SEQ ID NO: 23 - DNA, 89 bp, Unknown
  Genus Anterivirus of Arteriviridae
ggcctgtcgtccggcgacccgatcacctctgtgtctaacaccatttacagtttggtgatctacgcacagcatatggtgttcagttacat

SEQ ID NO: 24 - DNA, 88 bp, Unknown
  Genus Anterivirus of Arteriviridae
ggcctgtcgtctggcgacccgatcacctctgtgtctaacaccatttatagtttggtgatatatgcacagcatatggtgttcagttaca

SEQ ID NO: 25 - DNA, 89 bp, Unknown
  Genus Anterivirus of Arteriviridae
ggcctgtcgtccggcgacccgatcagctctgtgtctaacaccatttacagtttggtgatctacgcacagcatatcctcttcagttacat

SEQ ID NO: 26 - DNA, 88 bp, Unknown
  Genus Anterivirus of Arteriviridae
gttttcataaaaaaaggttagaaaataaaaataaaatctccatagctggtggttattttaaaagttagaatgagactataagcaatgg

SEQ ID NO: 27 - DNA, 89 bp, Unknown; identical to SEQ ID NO: 23
  Genus Anterivirus of Arteriviridae

SEQ ID NO: 28 - DNA, 89 bp, Unknown
  Genus Anterivirus of Arteriviridae
ggcctgtcgtctggcgacccgatcacctctgtgtctaacaccatttacagtttggtgatctacgcacagcatatggtgctcagttactt

SEQ ID NO: 29 - DNA, 28 bp, Unknown
  Chicken Infectious Bronchitis Virus (IBV)
ccgaattcgctatgaaaactgaacaaaa

SEQ ID NO: 30 - DNA, 28 bp, Unknown
  Chicken Infectious Bronchitis Virus (IBV)
gggtcgacatccataactaacacaaggg

SEQ ID NO: 31 - DNA, 20 bp, Unknown
  Chicken Infectious Bronchitis Virus (IBV)
tcaaagcttcanggngcgta

SEQ ID NO: 32 - DNA, 22 bp, Unknown
  Chicken Infectious Bronchitis Virus (IBV)
ctcgaattccngtrtaytgrca

SEQ ID NO: 33 - DNA, 18 bp, Unknown
  Chicken Infectious Bronchitis Virus (IBV)
gtattctgctttaaaaag

SEQ ID NO: 34 - DNA, 18 bp, Unknown
  Chicken Infectious Bronchitis Virus (IBV)
agctcaccactataaaca

SEQ ID NO: 35 - protein, 5 aa, Unknown
  Chicken Infectious Bronchitis Virus (IBV)
HGGAY

SEQ ID NO: 36 - protein, 14 aa, Unknown
  Chicken Infectious Bronchitis Virus (IBV)
SENNAGTAPSCTAG

SEQ ID NO: 37 - protein, 32 aa, Unknown
  Chicken Infectious Bronchitis Virus (IBV)
KSGSNSCPLTGLIPKGQIRISAMRSVNSRLHI

SEQ ID NO: 38 - protein, 6 aa, Unknown
  Chicken Infectious Bronchitis Virus (IBV)
CQYNTG

SEQ ID NO: 39 - protein, 13 aa, Unknown
  Chicken Infectious Bronchitis Virus (IBV)
TISYNAGTACTAG

SEQ ID NO: 40 - protein, 27 aa, Unknown
  Chicken Infectious Bronchitis Virus (IBV)
KAGSNGLIPKGQIRISAMRSVNSRLHL

SEQ ID NO: 41 - protein, 14 aa, Unknown
  Chicken Infectious Bronchitis Virus (IBV)
SENNAGTAPSCTAG

SEQ ID NO: 42 - protein, 32 aa, Unknown
  Chicken Infectious Bronchitis Virus (IBV)
KSGSNSCPLTGLIPKGQIRISAMRSVNSRLHF

SEQ ID NO: 43 - protein, 13 aa, Unknown
  Chicken Infectious Bronchitis Virus (IBV)
SENNAGAPSCTAG

SEQ ID NO: 44 - protein, 31 aa, Unknown
  Chicken Infectious Bronchitis Virus (IBV)
KSGSNSCPLTGLIPKGQIRIRMASVNSRLTI

SEQ ID NO: 45 - protein, 14 aa, Unknown
  Chicken Infectious Bronchitis Virus (IBV)
SENNAGTAPSCTAG

SEQ ID NO: 46 - protein, 32 aa, Unknown
  Chicken Infectious Bronchitis Virus (IBV)
KAGSNSCPLTGLIPKGQIRISAMRSVNSRLHI

SEQ ID NO: 47 - protein, 14 aa, Unknown
  Chicken Infectious Bronchitis Virus (IBV)
SENNAPTQYSCTAG

SEQ ID NO: 48 - protein, 30 aa, Unknown
  Chicken Infectious Bronchitis Virus (IBV)
KSGSNCPLTGLIPKGQIRISAMRSVNSRLF

SEQ ID NO: 49 - protein, 12 aa, Unknown
  Chicken Infectious Bronchitis Virus (IBV)
TISYTAPSCTAG

SEQ ID NO: 50 - protein, 28 aa, Unknown
  Chicken Infectious Bronchitis Virus (IBV)
ISGSNSCPLTGLIPRISAMRNVNSRLLI

SEQ ID NO: 51 - protein, 11 aa, Unknown
  Chicken Infectious Bronchitis Virus (IBV)
SEINAGSCTAG

SEQ ID NO: 52 - protein, 28 aa, Unknown
  Chicken Infectious Bronchitis Virus (IBV)
KAGSNSCLIPKGQIRISAMRSVNSRLHA

SEQ ID NO: 53 - DNA, 27 bp, Unknown
  Primer with NaeI restriction enzyme site to amplify HA gene
tctgccggcgagaaaatagtgcttctt

SEQ ID NO: 54 - DNA, 34 bp, Unknown
  Primer with HindIII restriction enzyme site to amplify HA gene
cccaagctttaaatgcaaattctgcattgtaacg

SEQ ID NO: 55 - DNA, 33 bp, Unknown
  Primer with SpeI restriction enzyme site to amplify pgsA gene from chromosomal DNA of Bacillus subtilis
ggactagtatgaaaaaagaactgagctttcatg

SEQ ID NO: 56 - DNA, 36 bp, Unknown
  Primer to amplify pgsA gene from chromosomal DNA of Bacillus subtilis
tcctcctggggatccagattttagtttgtcactatg

SEQ ID NO: 57 - DNA, 33 bp, Unknown
  Primer to amplify HA1 gene from A/chicken/ Henan/12/2004(H5N1)
ggatccccaggaggagatcagatttgcattggt

SEQ ID NO: 58 - DNA, 23 bp, Unknown
  Primer to amplify HA1 gene from A/chicken/ Henan/12/2004(H5N1)
cccaascttttatctcttttttc

(19) United States
(12) Patent Application Publication
(10) Pub. No.: US 2013/0004547 A1
(43) Pub. Date: Jan. 3, 2013

(54) ORAL VACCINES PRODUCED AND ADMINISTERED USING EDIBLE MICRO-ORGANISMS INCLUDING LACTIC ACID BACTERIAL STRAINS

(75) Inventors: Dominic Man-Kit LAM, Hong Kong (CN); Yuhong XU, Shanghai (CN)

(73) Assignee: VAXGENE CORPORATION, Hong Kong (CN)

(21) Appl. No.: 13/530,803

(22) Filed: Jun. 22, 2012

Related U.S. Application Data
(63) Continuation-in-part of application No. 13/383,864, filed on Jan. 13, 2012.
Application No. 13/383,864 is a continuation-in-part of application No. PCT/US10/62034, filed on Dec. 23, 2010.
(60) Provisional application No. 61/224,973, filed on Jul. 13, 2009.
(60) Provisional application No. 61/263,215, filed on Nov. 20, 2009.
(60) Provisional application No. 61/289,663, filed on Dec. 23, 2009.

(51) Int. Cl. A61K 39/00; A61P 37/04; A61K 9/50; A61K 39/39; A61K 39/12; A61K 39/145

(52) U.S. Cl. 424/400; 424/200.1

(57) ABSTRACT

The invention provides for anti-pathogen vaccines produced in recombinant bacteria and/or transgenic plants and administered through standard vaccine introduction methods or oral administration. In one embodiment, the vaccine is administered through the consumption of the edible plant as food, or the bacteria administered orally. The present invention also provides a method of using genetically modified microorganisms, such as lactic acid bacteria, including Lactococcus lactis strains, as oral vaccines. In one embodiment, Lactococcus lactis expressing the avian influenza HA gene can be used as an oral vaccine for protection against H5N1 virus infection. In another embodiment, said Lactococcus lactis is administered as an oral vaccine in conjunction with an adjuvant such as cholera toxin B.

This application is a continuation-in-part application of International Application No. PCT/US10/41792, filed Jul. 13, 2010, which claims the benefit of U.S. Ser. No. 61/263,215, filed Nov. 20, 2009, and U.S. Ser. No. 61/224,973, filed Jul. 13, 2009. This application is also a continuation-in-part application of International Application No. PCT/US10/62034, filed Dec. 23, 2010, which claims the benefit of U.S. Ser. No. 61/289,663, filed Dec. 23, 2009. The entire contents and disclosures of the preceding applications are incorporated by reference into this application.

Throughout this application, various references or publications are cited. Disclosures of these references or publications in their entireties are hereby incorporated by reference into this application in order to more fully describe the state of the art to which this invention pertains.

### FIELD OF THE INVENTION

This invention pertains to vaccines against animal viruses, bacteria, other pathogenic organisms and/or antigenic agents. This invention also concerns methods of preparing such vaccines. In one embodiment, the invention relates to edible plants expressing exogenous antigens and use of such plants as a vaccine. The invention further concerns expression of exogenous antigens in microorganisms such as bacteria, and use of such microorganisms as a vaccine. In one embodiment, the present invention provides compositions and methods of using genetically modified Lactococcus lactis strains as an oral vaccine. In another embodiment, the present invention provides for a method of using genetically modified Lactococcus lactis strains expressing the avian influenza HA gene as an oral vaccine for protection against H5N1 virus infection. In a further embodiment, the present invention provides for methods of using the aforementioned oral vaccine Lactococcus lactis strains for protection against H5N1 with a selected adjuvant, such as cholera toxin B. The invention further pertains to methods of preparing and administering such plant or microorganism derived vaccines.

### BACKGROUND OF THE INVENTION

Domestic animal diseases annually cause reductions in livestock of substantial proportions and have a drastic commercial impact. In developed countries where veterinary services and injected vaccines are more readily obtained, such diseases, while remaining a cause for concern, are often ameliorated. This is especially true due to larger herd/flock sizes and excess food-producing capability in these countries. In the lesser developed countries, the lack of veterinary services and drugs for such diseases and the reduced food-producing capacity has a much more substantial impact on the human population, leading to food shortages and human health problems.

The usefulness of antibiotics to effectively control bacterial pathogens is becoming increasingly difficult, because of the increased occurrence of antibiotic-resistant pathogens. Because of this, the voluntary reduction of antibiotic additives to animal feeds is practiced by many producers. However, since prevention of infectious diseases is more cost effective than the ultimate treatment of the disease once it has occurred, increased attention is being focused on the development of vaccines.

Vaccines are administered to animals to induce their immune systems to produce antibodies against viruses, bacteria, and other pathogenic organisms. In economically advanced countries, vaccines have brought many diseases under control. In particular, many viral diseases are now prevented due to the development of immunization programs.

But many vaccines for such diseases as rabies, foot and mouth disease, etc. are still too expensive for the lesser developed countries to provide to their large herd/flock animal populations. Lack of these preventative measures for animal populations routinely worsens the human condition by creating food shortages in these countries.

Microbial pathogens infect a host by: entering through a break in the integument induced by trauma; introduced by vector transmission; or by interacting with a mucosal surface.

The majority of animal pathogens initiate disease by the last mechanism, i.e., following interaction with mucosal surfaces. Bacterial and viral pathogens that act through this mechanism first make contact with the mucosal surface where they may attach and then colonize, or be taken up by specialized absorptive cells (M cells) in the epithelium that overlay Peyer's patches and other lymphoid follicles. Organisms that enter the lymphoid tissues may be readily killed within the lymphoid follicles, thereby provoking a potentially protective immunological response as antigens are delivered to immune cells within the follicles. Alternatively, pathogenic organisms capable of surviving local defense mechanisms may spread from the follicles and subsequently cause local or systemic disease (e.g. Salmonella spp.).

Most pathogens enter on or through a mucosal surface, with the exception of insect-borne pathogens or pathogens entering the body through wounds. Pathogens that enter through mucosal surfaces include, without limitation, Actinomyces, Aeromonas, Bacillus, Bacteroides, Bordetella, Brucella, Compylobacter, Capnbocylophaga, Clanrdia, Clostridium, Corynebacteriurn, Eikenella, Erysipelothriz, Escherichia, Fusobacterium, Hemophilus, Klebsiella, Legionella, Leptospira, Lisleria, Mycobacterium, Mcoplasma, Neisseria, Nocardia, Pasteurella, Proteus, Pseudomonas, Rickettsia, Salmonella, Selenomonas, Shigelia, Staphylococcus, Streptococcus, Treponema, Bibro, and Yersinia, pathogenic viral strains from the groups Adetiovirus, Coronavirus, Herpesvirus, Orthomyxovirus, Picornovirus, Poxvirus, Reovirus, Retrovirus, and Rotavirus, pathogen fungi from the general Aspercillus, Blastomyces, Candida, Coccoidiodes, Cryptococcus Histoplasma and Phycomyces, and pathogenic parasites in the general Eimeria, Entamoeba, and Trichomonas.

Mammalian hosts infected by a pathogen mount an immune response in an attempt to overcome the pathogen. The immune system consists of three branches: mucosal, humoral, and cellular. Mucosal immunity results from the production of secretory (sIgA) antibodies in secretions that bathe all mucosal surfaces including the respiratory tract, gastrointestinal tract, and the genitourinary tract and which are present in secretions from all secretory glands. Secretory IgA antibodies prevent colonization of pathogens on the mucosal surfaces and are a first line of defense against colonization and invasion of a pathogen through the mucosal surfaces. The production of sIgA can be stimulated whether by local immunization of the secretory gland or tissue or by presentation of an antigen to either the gut-associated lymphoid tissue (GALT or Peyer's patches) or the bronchial-associated lymphoid tissue (BALT).

Membranous microfold cells, otherwise known as M cells, cover the surface of the GALT and BALT and may be associated with other secretory mucosal surfaces. M cells act to sample antigens from the lumenal space adjacent to the mucosal surface and transfer such antigens to antigen-presenting cells (dendritic cells and macrophages), which in turn present the antigen to T lymphocytes (in the case of T-dependent antigens), which process the antigen for presentation to committed B cells. B cells are then stimulated to proliferate, migrate, and ultimately transform into antibody-secreting plasma cells producing IgA against the presented antigen.

When the antigen is taken up by M cells overlying the GALT and BALT, a generalized mucosal immunity results with sIgA against the antigen being produced by all secretory tissues in the body. Because most pathogens enter through mucosal surfaces and such surfaces make up the first line of defense to infection and facilitate the body's immune response, vaccines that can be orally administered represent an important route to stimulating a generalized mucosal immune response leading to local stimulation of a secretory immune response in the oral cavity and in the gastrointestinal tract.

Secretory IgA antibodies directly inhibit the adherence of microorganisms to mucosal epithelial cells and to the teeth of the host. This inhibition may be the result of agglutination of microorganisms, reduction of hydrophobicity or negative charge, and blockage of microbial adhesions. These anti-adherence effects are amplified by other factors such as secretory glycoproteins, continuous desquamation of surface epithelium and floral competition.

Clinical experience with human peroral poliovirus vaccine and several peroral or intranasal virus vaccines applied in veterinary medicine show that sIgA plays a decisive role in the protective effect by the mucosal immune system against respiratory and enteric viral infections. The effect of sIgA appears to be that of inhibiting the entry of viruses into host cells rather than prevention of attachment.

Secretory IgA antibodies directed against specific virulence determinants of infecting organisms play an important role in overall mucosal immunity. In many cases, it is possible to prevent the initial infection of mucosal surfaces by stimulating production of mucosal sIgA levels directed against relevant virulence determinants of an infecting organism. Secretory IgA may prevent the initial interaction of the pathogen with the mucosal surface by blocking attachment and/or colonization, neutralizing surface acting toxins, or preventing invasion of the host cells.

Parenterally administered inactivated whole-cell and whole-virus preparations are effective at eliciting protective serum IgG and delayed type hypersensitivity reactions against organisms that have a significant serum phase in their pathogenesis (e.g, human and animal pathogens such as Salmonella typhi and Hepatitis B). However, parenteral vaccines are not effective at eliciting mucosal sIgA responses and are ineffective against bacteria that interact with mucosal surfaces and do not invade (e.g., human and animal pathogens such as Vibrio cholerae).

Oral immunization can be effective for induction of specific sIgA responses if the antigens are presented to the T and B lymphocytes and accessory cells contained within the Peyer's patches where preferential IgA B-cell development is initiated. The Peyer's patches contain helper T cells (TH) that mediate B-cell isotype switching directly from IgM cells to IgA. B cells then migrate to the mesentric lymph nodes and undergo differentiation, enter the thoracic duct, then the general circulation, and subsequently seed all of the secretory tissues of the body, including the lamina propria of the gut and respiratory tract. IgA is then produced by the mature plasma cells, complexes with membrane-bound Secretory Component, and transported onto the mucosal surface where it is available to interact with invading pathogens. The existence of this common mucosal immune system explains in part the potential of live oral vaccines and oral immunization for protection against pathogenic organisms that initiate infection by first interacting with mucosal surfaces.

Because of the simplicity of oral delivery, there is great current interest in discovering new oral vaccine technology. Appropriately delivered oral immunogens can stimulate both humoral and cellular immunity and have the potential to provide cost-effective, safe vaccines for use in developing countries where large-scale parenteral immunization of herd or other commercially produced animals is not practical or extremely difficult to implement. Such vaccines may be based upon bacterial or viral vector systems expressing protective epitopes from diverse pathogens (multivalent vaccines) or may be based upon purified antigens delivered singularly or in combination with relevant antigens or other pathogens.

A number of strategies have been developed for oral immunization, including the use of attenuated mutants of bacteria (e.g., Salmonella spp.) as carriers of heterologous antigens, encapsulation of antigens into microspheres composed of poly-DL-lactide-glycolide (PGL), protein-like polymer-proteinoids, gelatin capsules, different formulations of liposomes, adsorption onto nanoparticles, use of lipophilic immune stimulating complexes, and addition of bacterial products with known adjuvant properties.

Underlying the development of most current vaccines is the ability to grow the disease-causing agent in large quantities. At present, vaccines are usually produced from killed or live attenuated pathogens. If the pathogen is a virus, large amounts of the virus must be grown in an animal host or cultured animal cells. If a live attenuated virus is utilized, it must be clearly proven to lack virulence while retaining the ability to establish infection and induce humoral and cellular immunity. If a killed virus is utilized, the vaccine must demonstrate the lack of capacity of surviving antigens to induce immunization. Additionally, surface antigens, the major viral particles that induce immunity, may be isolated and administered to induce immunity in lieu of utilizing live attenuated or killed viruses.

Vaccine manufacturing often employs complex technologies entailing high costs for both the development and production of the vaccine. Concentration and purification of the vaccine is required, whether it is made from cell cultures, whole bacteria, viruses, other pathogenic organisms or sub-units thereof. Even after these precautions, problems can and do arise. With killed bacterial cells, viruses or other pathogenic organisms, there is always a chance that live pathogens survive and vaccination may lead to isolated cases of the disease. Moreover, the vaccines may sometimes be contaminated with cellular material from the culture material from which it was derived. These contaminates can cause adverse reactions in the vaccine recipient animal and sometimes even death.

Direct injection of plasmid DNA has been used as a vaccine strategy, with reports of protective immunity and cytotoxic T lymphocyte (CTL) induction in mice after i.m. injection of a DNA plasmid. The use of DNA vaccines in preclinical studies has become well established, with reports of protective immunity in many different independent studies. In recent studies, both antibody and CTL responses were induced in non-human primates, although 1-2 mg of DNA was immunized on multiple occasions in these studies. However, the use of very high doses of DNA is less favorable from a process economics standpoint; therefore, there is a clear need to induce effective immunity in veterinary medicine with lower and fewer doses of DNA, as well as to increase the magnitude of the immune responses obtained.

A number of strategies available that have the potential to improve the potency of DNA vaccines include vector modification to enhance antigen expression, improvements in DNA delivery, or the inclusion of adjuvants. It has been reported that the monophoryl lipid A could enhance both humoral and cell-mediated immune responses to DNA vaccination against human immunodefciency virus type 1 (Shin et al., 1997). DNA vaccines formulated with QS-21 saponin adjuvant administered via intramuscular and intranasal routes could also induce systemic and mucosal immune responses. (Shin S, et al., 1998). Manmohan S had developed a delivery system for DNA vaccines-cationic microparticles. Vitamin D3 also plays an important role in immunization via DNA vaccine.

It is also reported that bacterial DNA-sequences called immunostimulatory sequences can be a potent adjuvant. Non-methylated, palindrome DNA-sequences containing CpG-oligodinucleotides (CpG-ODN) can activate an innate immune response by activating monocytes, NK cells, dendritic cells and B-cells in an antigen-independent manner (immunostimulatory DNA sequences, ISS). Methylation of the CpG-ODN reportedly abrogates the immunogenicity of the DNA vaccine. The use of large amounts of plasmid for immunization might only overcome the low transfection efficiency in vivo, as well as serve as an adjuvant, driving a Th1-type response.

The propagation of highly pathogenic avian influenza (HPAI) H5N1 virus remains a major concern globally. In addition to the many outbreaks reported annually in various bird populations all over the world, more than 385 human cases of H5N1 infection have also been reported. Subbarao, K. et al., 1998, Science 279, 393-396. This virus strain is highly susceptible to antigen drift and has already caused several outbreaks in human subjects with a very high mortality rate. Subbarao, K. et al., 1998, Science 279, 393-396. Vaccination is considered the most desirable counteraction to prevent the spreading and rapid mutation of the virus. It is also highly preferable to develop vaccines for all the species affected to slow down cross-species spreading. Therefore it is of prominent importance to develop vaccines to help contain viral contagion in animals and deter further development into a major threat and health crisis for human beings.

There have been many approaches taken to make influenza viral vaccines. The most commonly used strategy is to administer heat inactivated whole viruses grown in embryonated eggs. But the production of egg-derived vaccines against the deadly H5N1 viruses has not proven very effective; moreover, a protective immune response has only been elicited upon the administration of large doses of inactivated whole viruses produced in this fashion. Seroconversion rates and the magnitude of immune response were suboptimum after administration of egg-derived vaccines. See, e.g. Johansson, B. E. et al., 2006, N. Engl. J. Med. 354:2724-2725; Kilbourne, E. D. et al., 2002, Proc. Natl. Acad. Sci. USA 99:10748-52; Wei, C. J. et al., 2008, J. Virol. 82:6200-6208.

Other more advanced approaches have included using recombinant subunit vaccines produced in a baculovirus expressing system (Chen, M. W., et al., 2008, Proc. Natl. Acad. Sci. USA 105:7235-7240), plasmid DNA vaccines (Kong, W. P., et al., 2006, Proc. Natl. Acad. Sci. USA 103:15987-15991; Hoelscher, M., et al., 2006, Lancet 367:475-481; Gao, W., et al., 2006, J. Virol. 80:1959-1964) and replication-incompetent adenovirus vector vaccines (Mason, H. S., et al. 1992, Proc. Natl. Acad. Sci. USA 89:11745-11749; Lam, D. M. K. & Shi, J., 1996, Agro-Food-Industry high-tech March/April, 7-12; Lam, D. M. K., et al., 1997, Foreign Medical Sciences China 20: 145-149; Nochi, T., et al., 2007, Proc. Natl. Acad. Sci. USA 104, 10986-10991).

Each showed promise in protecting mice against lethal viral challenges (Kong, W. P., et al., 2006, Proc. Natl. Acad. Sci. USA 103:15987-15991; Hoelscher, M., et al., 2006, Lancet 367, 475-481). However, all such vaccines were designed to be administered intramuscularly, presenting practical difficulties in respect of administration to large populations of animals.

Therefore a practical and effective influenza vaccine that can be easily applied to humans and animals is more highly desirable if the vaccine can be administered with food. The use of edible plants as expression and delivery vehicles for vaccines offers many advantages for a variety of infectious diseases, especially those whose pathogens are relatively conserved. This is because it normally takes over 1 year to produce stably expressed transgenic plants. In contrast, for diseases in which the infective viruses mutate or shift rapidly, such as the influenza viruses, expression vehicles that are both edible and can be transformed in a matter of weeks are much more desirable. It is mainly for this reason that the lactic acid bacteria (LAB) was chosen.

Towards this end, the present invention uses the Lactococcus lactis (L. lactis) vector system which can be safely administered orally. Lactococcus lactis is a Gram-positive lactic acid bacterium that is widely used for the production and preservation of fermented milk products, which is generally regarded as safe (GRAS). It can be engineered to express various proteins, including bacterial and viral antigens (Xin, K. Q., et al., 2003, Blood 102, 223-228; Robinson, K. et al., 1997, Nat. Biotechnol. 15:653-657; Gilbert, C. et al., 2000, Infect. Immun 68:3251-3260; Cho, H. J. et al., 2007, Vaccine 17:607-616; Klijn, N. et al., 1995, Appl. Environ. Microbiol. 61:2771-2774).

Mice given these vectors generated antigen specific mucosal as well as systemic immune responses (Xin, K. Q. et al., 2003, Blood 102:223-228; Robinson, K. et al., 1997, Nat. Biotechnol. 15:653-657; Klijn, N. et al., 1995, Appl. Environ. Microbiol. 61: 2771-2774). This vaccine approach has been reported in studies using recombinant L. lactis encoding various antigens for oral administration (Benfield D. A., et al., 1992, J Veterinary Diagnostic Investigation 4:127-133; Benfield, D. A., et al., 1992, Diseases of Swine 7th Ed. Ames, Iowa: Iowa State University Press: 756-762; Bresson J. L., et al. Safety and immunogenicity of an inactivated split-virion influenza A/Vietnam/1194/2004 (H5N1) vaccine: phase I randomised trial. Lancet 2006, 367:1657-64; Cavanagh, D. 1997, Arch of Virol. 142: 629-633), resulting in a variety of immune responses thought to be related to the type of antigen, the amount of antigen expressed, and the duration of antigen expression in the gut (Klijn, N. et al., 1995, Appl. Environ. Microbiol. 61:2771-2774; Gruzza, M. et al., 1994, Microb. Releases 2:183-189). However, antigen inoculation efficiency remains low because most of the organisms cannot survive the harsh acidic environment of the stomach and protease degradation in the GI tract (Rowe, T. et al., 1999, J. Clin. Microbiol. 37, 937-943).

### Plant Genetic Engineering

Various methods are known in the art to accomplish the genetic transformation of plants and plant tissues so that foreign DNA is introduced into the plant's genetic material in a stable manner, i.e., a manner that will allow the foreign DNA to be passed on to the plant's progeny. Two such transforming procedures are Agrobacterium-mediated transformation and direct gene transfer.

Agrobacterium-mediated transformation utilizes A. tumefaciens, the etiologic agent of crown gall, a disease of a wide range of dicotyledons and gymnosperms that results in the formation of tumors or galls in plant tissue at the site of infection. Agrobacterium, which normally infects the plant at wound sites, carries a large extrachromosomal element called Ti (tumor-inducing) plasmid.

Ti plasmids contain two regions required for tumor induction. One region is the T-DNA (transferred-DNA) which is the DNA sequence that is ultimately found stably transferred to plant genomic DNA. The other region is the vir (virulence) region which has been implicated in the transfer mechanism. Although the vir region is absolutely required for stable transformation, the vir DNA is not actually transferred to the infected plant. Transformation of plant cells mediated by infection with A. tumefaciens and subsequent transfer of the T-DNA alone have been documented. See, e.g., Bevan, M. W. et al. Int. Rev. Genet. 16, 357 (1982).

After several years of intense research in many laboratories, the Agrobacterium system has been developed to permit routine transformation of a variety of plant tissues. Representative tissues transformed by this technique include, but are not limited to, tobacco, tomato, sunflower, cotton, rapeseed, potato, poplar, and soybean.

A. rhizogenes has also been used as a vector for plant transformation. That bacterium, which incites root hair formation in many dicotyledonous plant species, carries a large extrachromosomal element called a Ri (root-inducing) plasmid which functions in a manner analogous to the Ti plasmid of A. tumefaciens. Transformation using A. rhizogenes has developed analogously to that of A. tumefaciens and has been successfully utilized to transform plants that include but are not limited to alfalfa and poplar.

In the case of direct gene transfer, foreign genetic material is transformed into plant tissue without the use of the Agrobacterium plasmids. Direct transformation involves the uptake of exogenous genetic material into plant cells or protoplasts. Such uptake may be enhanced by use of chemical agents or electric fields. The exogenous material may then be integrated into the nuclear genome. The early work with direct transfer was conducted in the dicot Nicotiana tabacum (tobacco) where it was shown that the foreign DNA was incorporated and transmitted to progeny plants. Several monocot protoplasts have also been transformed by this procedure including maize and rice.

Liposome fusion has also been shown to be a method for transforming plant cells. Protoplasts are brought together with liposomes carrying the desired gene. As membranes merge, the foreign gene is transferred to the protoplast.

In addition, direct gene transfer can be accomplished by polyethylene glycol (PEG) mediated transformation. PEG mediated transformation has been successfully used to transform dicots such as tobacco and monocots such as lolium multiflorum. This method relies on chemicals to mediate the DNA uptake by protoplasts and is based on synergistic interactions between Mg+z, PEG, and possibly Ca+z. See, e.g., Negrutiu, R. et al., Plant Mal. Biol. 8, 363 (1987).

Alternatively, exogenous DNA can be introduced into cells or protoplasts by microinjection. In this technique, a solution of the plasmid DNA or DNA fragment is injected directly into the cell with a finely pulled glass needle. This technique has been used to transform alfalfa.

A more recently developed procedure for direct gene transfer involves bombardment of cells by micro-projectiles carrying DNA. In this procedure, commonly called particle bombardment, tungsten or gold particles coated with the exogenous DNA are accelerated toward the target cells. The particles penetrate the cells carrying with them the coated DNA. Microparticle acceleration has been successfully demonstrated to lead to both transient expression and stable expression in cells suspended in cultures, protoplasts, immature embryos of plants including but not limited to onion, maize, soybean, and tobacco.

Once plant cells have been transformed, there are a variety of methods for regenerating plants. The particular method of regeneration will depend on the starting plant tissue and the particular plant species to be regenerated. In recent years, it has become possible to regenerate many species of plants from callus tissue derived from plant explants. The plants that can be regenerated from callus include monocots, such as but not limited to com, rice, barley, wheat, and rye, and dicots, such as but not limited to sunflower, soybean, cotton, rapeseed and tobacco.

Regeneration of plants from tissue transformed with A. tumefaciens has been demonstrated for several species of plants. These include but are not limited to sunflower, tomato, white clover, rapeseed, cotton, tobacco, potato, maize, rice, and numerous vegetable crops.

Plant regeneration from protoplasts is occasionally a useful technique. When a plant species can be regenerated from protoplasts, then direct gene transfer procedures can be utilized, and transformation is not dependent on the use of A. tumefaciens. Regeneration of plants from protoplasts has been demonstrated for plants including but not limited to tobacco, potato, poplar, corn, and soybean.

The technology developed for the creation of transgenic plants has led many investigators to study the expression of genes derived from dissimilar plant species or from non-plant genomes. In many cases, it has been desirable to characterize the expression of recombinant proteins encoded by genes derived from viruses or bacteria. The construction of chimeric genes for expression of foreign coding sequences in plants involves ligation of non-coding regulatory elements which function in plants 5′ to the DNA sequence encoding the desire protein, and ligation of a polyadenylation signal which is active in plant cells 3′ to the DNA sequence encoding the desired protein.

The 5′ regulatory sequences which are often used in the creation of chimeric genes for plant transformation may cause either nominally constitutive expression in all cells of the transgenic plant, or regulated gene expression where only specific cells or tissues show expression of the introduced genes. The CaMV 35-S promoter, which was derived from the Cauliflower Mosaic Virus that causes a plant disease, has frequently been used to drive nominally constitutive expression of foreign genes in plants. A regulatory DNA element which was found to control the tuber-specific expression of the patatin protein is an example of developmentally specific gene expression; this patatin promoter element is known to cause the tuber-specific expression of at least some foreign genes. See, e.g., H. C. Wenzler, et al., 1989, Plant Mol. Biol. 12:41-50.

Chimeric gene constructions may also include modifications of the amino acid coding sequence of the structural gene being introduced into transgenic plants. For example, it may be desirable to add or delete amino acids in the protein to be expressed to influence the cellular localization of foreign gene product in the cells of transgenic plants.

It has been shown that the inclusion of KDEL (SEQ ID NO: 1) and HDEL (SEQ ID NO:32) amino acid sequences at the carboxy-terminus of at least one protein enhanced the recognition for that protein by the plant endoplasmic reticulum retention machinery. S. Munro and H. R. B. Pelham, 1987, Cell 48:988-997; J. Denecke, et al., 1992, EMBO-J 11:2345; E. M. Herman, et al., 1991, Planta 182:305; C. Wandelt, et al., 1992, The Plant Journal 2:181. However, such modifications are problematic at best because other factors such as protein conformation or protein folding in the transformed cells may interfere with the availability of this carboxy terminus signal by the plant endoplasmic reticulum retention machinery. S. M. Haugejorden, et al., 1991, J Biol. Chem. 266:6015.

### Oral Vaccine Methodologies Using Transgenic Plants

The high cost of production and purification of synthetic peptides manufactured by chemical or fermentation based processes may prevent their broad scale use as oral vaccines. The production of immunogenic proteins in transgenic plants and the adjuvant effect of such proteins in transgenic plants offer an economical alternative.

While oral vaccines may be an effective and inexpensive procedure for inducing secretory immune responses in animals including humans, there is a need for proven techniques that yield transgenic plants or plant tissue that can, upon direct ingestion, cause a desired immune response to a given antigen without significant side effects.

Attempts to produce transgenic plants expressing bacterial antigens of E. coli and of Streptococcus mutants have been made. Curtiss and lhnen, WO 90/0248, published Mar. 22, 1990. Transgenic plants that express the Hepatitis B surface antigen (HBsAg) have also been made. H. S. Mason, et al., Proc. Nat. Acad. Sci. USA, 89:11745-749 (1992).

A series of patents issued to at least certain of the present inventors in the United States have been directed at producing vaccines in plants. In U.S. Pat. No. 5,484,719 (Lam, Arntzen) issued Jan. 16, 1996, a plasmid vector was described comprising recombinant hepatitis B viral surface antigen protein DNA and a plant-functional promoter capable of directing the synthesis of the cloned protein in the plant. That patent also disclosed the use of the disclosed plasmid for constructing a transgenic tobacco plant cell. In U.S. Pat. No. 5,612,487 (Lam, Arntzen) issued Mar. 18, 1997, a transgenic tobacco plant was described comprising a recombinant hepatitis B viral surface antigen protein in which the plant was capable of synthesizing the viral protein into antigenic particles. That patent also disclosed a method for producing an antigenic composition using the transformed tobacco plant from which the antigenic particles were recovered to be used as a vaccine. In both of these disclosures, the inventors recognized the limitations imposed with using the tobacco plant as a host for the recombinant vaccine. Tobacco alkaloids and other toxic substances require substantial purification of the vaccine.

In U.S. Pat. No. 5,914,123 (Arntzen, Lam) issued Jun. 22, 1999, foods were disclosed comprising transgenic plant material capable of being ingested for its nutritional value, expressing a recombinant immunogen from Hepatitis virus or from Transmissible Gastroenteritis Virus.

In U.S. Pat. No. 6,034,298 (Lam, Arntzen, Mason) issued Mar. 7, 2000, a transgenic plant, as well as plasmids and methods for producing same, was disclosed expressing a recombinant viral antigenic protein from Transmissible Gastroenteritis virus. Also disclosed in that patent was a vaccine, as well as methods, for producing a pharmaceutical vaccine composition against Transmissible Gastroenteritis virus.

In U.S. Pat. No. 6,136,320 (Arntzen, Lam/Prodigene) issued Oct. 24, 2000, the following was disclosed: an orally acceptable immunogenic composition comprising unpurified or partially purified recombinant viral immunogen expressed in a plant, wherein said immunogen is expressed in the plant at a level such that upon oral administration of said composition to an animal, an immunogenic response is observed, and particularly said viral immunogen being an immunogenic protein from a virus selected from the group consisting of transmissible gastroenteritis virus and hepatitis virus, and more particularly a vaccine comprising an immunogen of hepatitis virus expressed in a plant, wherein said immunogen is capable of binding a glycosylated molecule on a surface of a membrane mucosal cell. Also disclosed was a plant composition comprising a viral antigen which triggers production of antibodies and which is derived from a hepatitis B virus surface antigen or transmissible gastroenteritis virus spike protein, and plant material; said antigen being a product produced by the method of expressing said immunogen in a transgenic plant, said plant material being in a form chosen from the group consisting of a whole plant, plant part, or a crude plant extract. Further disclosed was an anti-transmissible gastroenteritis vaccine comprising the composition of claim 8 wherein said antigen is derived from transmissible gastroenteritis virus spike protein.

In U.S. Pat. No. 6,194,560 (Arntzen, Mason, Haq/TAMU) 02.27.01, a synthetic E. coli gene which encodes LT-B, wherein said gene comprises the DNA sequence optimized for plant codon usage, was disclosed. As noted in that patent, those studies have not yielded orally immunogenic plant material. They have not demonstrated that it is, in fact, possible to orally immunize animals with antigens produced in transgenic plants.

### Animal Diseases

Hog cholera (HC), also known as classical swine fever, is a severe systemic and hemorrhagic disease in swine caused by Hog Cholera Virus (HCV). Classical swine fever or hog cholera represents an economically important disease of swine in many countries worldwide. Under natural conditions, the pig is the only animal known to be susceptible to HC. Hog cholera is a highly contagious disease that causes degeneration in the walls of capillaries, resulting in hemorrhages and necrosis of the internal organs. In the first instance hog cholera is characterized by fever, anorexia, vomiting and diarrhea which can be followed by a chronic course of the disease characterized by infertility, abortion and weak off-springs of sows. Nearly all pigs die within 2 weeks after the first symptoms appear.

HC can be transmitted from the infected swine to the healthy one by direct contact. The disease can also be transmitted through contact with body secretions and excrement from infected animals. Flies, birds and human can act as vectors in transmitting the virus.

While hog cholera does not cause food-borne illness in people, it causes serious economic losses to the pig industry since it can result in widespread deaths in pigs. Traditionally, HC syndrome is an acute disease of high morbidity and mortality. From time to time, evolution of the virus has led to a higher incidence of subacute and chronic forms.

Virulent strains induce an acute disease that is characterized by persistent fevers that can raise body temperatures as high as 107° F. Other signs of the acute form include convulsions, anorexia, leukopenia, tonsillar necrosis and lack of appetite. At day 3-4 post-infection, there is a generalized viremia with the virus replicating in epithelial cells, endothelial cells and cells of the mononuclear phagocyte system. Degeneration and necrosis of endothelial cells leads to vascular compromise, ischemia, and the induction of disseminated intravascular coagulation. These vascular changes result in petechial hemorrhage of the kidneys, urinary bladder and gastric mucosa, splenic infarction and lymph node hemorrhage. Death usually occurs within 5 to 14 days following the onset on illness.

The chronic form of hog cholera causes similar clinical signs in affected swine, but there is less hemorrhage associated. Discoloration of the abdominal skin and red splotches around the ears and extremities often occur. Pigs with chronic hog cholera can live for more than 100 days after the onset of the infection.

The mild or clinically unapparent form of hog cholera seldom results in noticeable clinical signs. Affected pigs suffer short periods of illness often followed by periods of recovery. The mild strain may cause small litter size, stillbirths and other reproductive failure. High morality during weaning may also indicate the presence of this mild strain of hog cholera.

When pregnant sows are infected with strains of lesser virulence, transplacental infection may occur. Depending on the stage of gestation, congenital infection can result in abortion, fetal mummification, stillbirths and embryonic malformations. The most frequent outcome with low virulence strains is the birth of persistently infected piglets in a state of immunological tolerance and shedding large quantities of virus.

Hog Cholera Virus (HCV) is a member of the Pestivirus genus of the Flaviviridae family (Francki, R. I. B. et al., 1991, Arch of Virol Supp 2:223-233; Horzinek, M., 1991, Arch of Virol Supp 3:1-5; Collett, M. S., 1992, Comparative Immunology, Microbiology and Infectious Diseases 15: 145-154). It replicates principally in lymphocytes and vascular endothelium. Replication of most of the HC strains is restricted to the cytoplasm of the cell and does not result in cytopathic effect.

Hog cholera virus has been shown to be structurally and serologically related to bovine viral diarrhea virus (BVDV) of cattle and to border disease virus (BDV) of sheep, which also belongs to the genus pestivirus within the family togaviridae. HCV is a small single positive-stranded RNA virus with a genome of approximately 12.3 kb (Vanderhallen, H., et al., 1999, Arch Vii al 144: 1669-1677). Its genome contains a single long open reading frame (ORF) that is flanked by a 5′ and 3′ nontranslated region (NTR). Meanwhile, it lacks both 5′ cap and a significant 3′ poly(A) sequences, if it possesses any polyadenylation. The HCV is believed to encode 3-5 structural proteins of which two are possibly glycosylated. The number of non-structural viral proteins is not known. Modified HCV vaccines (comprising attenuated or killed viruses) for combating hog cholera infection have been developed and are presently used. However, infection of tissue culture cells to obtain HCV material to be used in modified virus vaccines leads to low virus yields and the virions are very difficult to purify. Modified live virus vaccines always involve the risk of inoculating animals with partially attenuated pathogenic HCV which is still pathogenic and can cause disease in the inoculated animal or offspring and of contamination by other viruses in the vaccine. In addition the attenuated virus may revert to a virulent state. There are also several disadvantages using inactivated vaccines, e.g., the risk of only partial inactivation of viruses, the problem that only a low level of immunity is achieved requiring additional immunizations and the problem that antigenic determinants are altered by the inactivation treatment leaving the inactivated virus less immunogenic. The usage of modified HCV vaccines is not suited for eradication programs.

Vaccines containing only the necessary and relevant HCV immunogenic material that is capable of eliciting an immune response against the pathogen do not have the disadvantages of modified vaccines. Classically derived and administered recombinant HCV vaccines have been disclosed that contain only certain immunogenic portions of HCV. See, e.g., U.S. Pat. Nos. 5,935,582 (issued Aug. 10, 1999), 5,925,360 (issued Jul. 20, 1999), and 5,811,103 (issued Sep. 22, 1998) to Meyers et al.

The cDNA sequence derived from the genomic RNA of HCV is a continuous sequence about 12,500 nucleotides in length. It contains one long open reading frame (ORF), starting with the ATG codon at position 364 to 366 and ending with a TGA codon as a translational stop codon at position 12058 to 12060. This ORF consists of 3898 codons capable of encoding 435 kDa of protein.

In vivo, during HCV replication in an infected cell, this protein is synthesized as a polyprotein precursor molecule that is subsequently processed to fragment polypeptides by (enzymatic) cleavage of the precursor molecule. These fragments form after possible post-translational modifications of the structural and non-structural proteins of the virus. It is possible to derive a sequence that contains the genetic information for such a fragment with immunizing properties against HCV or immunological properties characteristic for HCV or containing the genetic information for a portion of such a fragment that still has the immunizing properties or the immunological properties characteristic for HCV.

Fragment polypeptides are located within the amino acid position at about 1-249, 263-487, 488-688 or 689-1067. The 1-249 region essentially represents the core protein whereas the 263-487, 488-688 and 689-1067 regions essentially represent glycoproteins of 44/48 kD, 33 kD and 55 kD respectively.

HCV is 40-50 nm in diameter. It has a nucleocapsid of about 29 nm. There are fringelike projections of 6-8 nm on the surface of the virion. The buoyant density, depending on the gradient material and on the cells used to propagate the virus, has been reported between 1.12 g/ml and 1.17 g/ml.

HCV is stable at pH 5-10; but above and below these pH values, infectivity is rapidly destroyed. HCV is quickly made inactive by lipid solvents, such as ether, chloroform and deoxycholate. Although its infectivity is lost in cell culture medium at 60° C. after 10 minutes, the virus is still active in defibrinated blood at 68° C. after 30 minutes. Moreover, the virus can survive in frozen carcasses for long periods of time and it can remain infective in pork and pork product for months, so it is of great epizootiologic importance.

All HC strains discovered so far were clustered into two main groups and five subgroups when genomic elements of the 5′ nontranslated region (Hofmann, M. A. et al., 1994, Arch Virol 139: 217-229), E₂ gene (Lowings, J. P. et al., 1994, J. Gen Virol 75: 3461-3468; Lowings, P. et al., 1996, J Gen Virol 77:1311-1321) or the NS5B gene (Vilcek, S. et al., 1996, Virus Res 43: 137-147) were compared.

The 5′ to 3′ genomic organization of HCV includes a nonstructural protein designated N, an encoded nucleocapsid protein designated C, a structural envelope associated glycoproteins (E) designated EO, E1 and E₂, nonstructural proteins (NS), designated NS2, N83, NS4A, NS4B, NSSA and NSSB.

The resulting polyprotein of about 3900 amino acids is co- and post-translationally processed by viral as well as host cellular proteases to yield four structural and seven to eight nonstructural viral proteins (Thiel, H-J. et al., 1996, Fundamental Virology, 3rd ed. Raven Press New York, 1059-1079).

Nucleocapsid protein C and the three envelope-associated glycoprotein ED (gp44/48), E1 (gp33) and E₂ (gp55) are the structural components of HCV. They are located within the N-terminal third of the polyprotein (Stark, R., et al., 1990, Virol 174: 286-289). The pestiviral capsid protein is preceded by a nonstructural protein, p23, in the polyprotein.

This non-structural core protein is a putative protease which exhibits autoproteolytic activity (Thiel et al., 1991, J. Virol 65: 4705-4712; Wiskerchen, M. A., et al., 1991, J Virol 65: 4508-4514).

EO lacks a typical membrane anchor and is secreted in considerable amounts from the infected cells (Rumenapf, T. et al., 1993, J Vrol, 67:3288-3294). Although this protein exhibits RNase activity, its enzymatic action for the viral lifecycle is still unknown.

E₂ and to a lesser extent, EO were found to be the targets for triggering neutralizing antibodies against the virus. On the other hand, E1 is believed to be buried in the viral envelope (Weiland, E. et al., 1990, J Virol 64: 3563-3569), and hardly any anti-E1 antibodies have been described. In the virions and in the infected cells, the glycoproteins form disulfide-linked complexes, such as: EO homodimer with a size of 100 kDa; E1-E₂ heterodimer with a size of 75 kDa; and E₂ homodimer with a size of 100 kDa (Thiel, H-J, et al., 1991, J Virol 65:4705-4712).

Infectious bronchitis (IB) is an acute, highly contagious viral respiratory disease of chickens characterized by tracheal rates, coughing and sneezing. The poultry industry in Southern China has experienced severe outbreaks of IB every year, particular in recent years; other viral respiratory diseases have also had a high incidence in the Pearl River delta region of China, and consequently, accurate and rapid genotyping is an important factor in controlling infectious bronchitis. The causative agent of IB is infectious bronchitis virus (IBV)5 which is classified in the coronaviridae family, genus coronaviras, with more than 20 serotypes identified in the world. Although vaccination with Mass-typed vaccine is widely used in China, outbreaks were still reported each year. IBV usually damages the respiratory tract, but strains of IBV replicating in the kidney, oviduct, intestine and glands have also been reported in China.

The IBV encodes three major structural proteins: the nucleocapsid protein (N), the membrane glycoprotein and S protein. The S protein can be cleaved post-translationally to release the N-terminal S-1 and C-terminal S-2 protein. The N-terminal subunit (S-I) is responsible for cell attachment, and determines tissue tropism and virus-neutralizing antibody induction, whereas the C-terminal subunit (S-2) anchors S-I to the viral envelope. The N-terminal part of the S-1 protein is variable between different serotypes and between different strains of the same serotype. Keeler and Kingham had identified two hypervariable regions (HVRs) and two conserved regions in the N-terminal part of the S-I gene in Mass sera-typed IBV. The HVRs that contain neutralization epitopes may be located in amino acid regions 56-69 and 117-137. The two conserved regions are located at 43-47 and 229-236. Previously HI and VN were often adopted in the diagnosis of IBV, but these methods had many drawbacks, such as being time and labor consuming; furthermore HI is not so reliable.

Since Jungher et al. first described antigenic differences between the Mass and Conn, the extensive antigenic diversity of IBV is well recognized. Due to immune selective pressures associated with intensive IBV vaccination and other poor bio-security practice, over 20 serotypes of the virus had been reported, and additional variant serotypes continue to emerge and cause disease.

Due to farm practice, mass-typed vaccine is widely used in China. In recent years, intensive industrial poultry farming industry in Southern China increased the immune selective pressure of vaccination; at the same time, other sources of IBV vaccine have also been brought into China due to an open door policy. The recombination between field strains and vaccine strains will also cause outbreaks of IBV.

Another commercially expensive disease is Infectious Bursal Disease (IBD), also known as Gumboro disease. IBD is caused by Infectious Bursal Disease Virus (IBDV). Apart from direct contact with infected birds, it is possible that rats and mosquitoes can transmit the disease.

During the 63rd General Session of the Office International des Epizooties (OIE Paris, 15 to 19 May 1995), it was estimated that IBD has considerable socio-economic importance at the international level, as the disease is present in more than 95% of the Member Countries (Eterradossi, N. et al., 1995; Paris OIE).

Although IBDV does not infect humans, it can cause severe economic loss. The economic importance of the disease can be categorized into two main aspects. First, some virus strains may cause up to 20% mortality in chickens three weeks of age or older; secondly, the virus may cause prolonged immunosuppression of chickens infected at an early age.

Chickens from 3 to 6 weeks of age are most susceptible to IBDV infection and mortality may be high. The syndrome of IBD can be clinical or subclinical. The age susceptibility is broader in the case of very virulent IBDV strains (Van den Berg T P, 1991, Avian Pathol. 20:133-143; Nunoya T. et al., 1992, Avian Drs. 36:597-609). After a short period of time (within 24 hours after infection), the bursa of Fabricius shows lesions. There is gelatinous yellowish transudate covering the surface of the bursa.

At day 3 post-infection, due to edema and hyperemia, the bursa increases in size and weight. At day 4 of post-infection, the size of the bursa has usually doubled. Later, the bursa recedes in size and the transudate disappears in the subsequent days. At day 8 of post-infection, the bursa usually becomes one-third of the original weight. There are also necrotic foci and petechial hemorrhages on the mucosal surface.

Due to impairment of clotting mechanisms, the pectoral muscle of the infected subject becomes dehydrated with darkened dislocations. Lesions and hemorrhage of other major organs, for instance, kidney, spleen, thymus and the harderian gland, can also be observed.

Instead of exhibiting clinical signs, prolonged immunosuppression arises in chickens at an early age (usually below three weeks of age) after catching IBD. As a result, the infected subject is more susceptible to other diseases. This predisposes the birds to other diseases such as colisepticaemia coccidiosis, infectious laryngotracheitis, infectious bronchitis and salmonellosis and colibacillosis. Moreover, lower antibody responses to vaccinations to other pathogen would result. In addition to the dominant suppression of the humoral immunity of infected chickens, cell-mediated immunity is also suppressed transiently.

IBDV is a member of the genus Birnavirus of the Birnaviridae family. It primarily infects lymphoid cells, especially precursor B cells. The primary target organ of the virus, the bursa of Fabricus, is the most severely affected.

IBDV is a single shelled, non-enveloped virion with icosahedral symmetry composed of 32 capsomers; it is 60 nm-70 nm in diameter. The capsid symmetry is askew. Buoyant density of the complete IBDV particle in cesium chloride gradient range from 1.31 g/ml to 1.34 g/ml.

IBDV resists treatment with ether and chloroform. It would be unaffected at pH 2. It is still viable when exposed at 56° C. for five hours. Moreover, the virus is unaffected by exposure to 0.5% phenol and 0.125% thimerosal at 30° C. for one hour. On the other hand, it would be inactivated at pH 12. Its infectivity would be reduced considerably when exposed to 0.5% formalin for six hours. It would even be killed when incubating at 70° C. for 30 minutes.

Two serotypes of IBDV were recognized and there were several strains within each serotype. Although its presence will stimulate antibodies, type II virus does not cause clinical disease. Hence, IBD vaccines have only been made from type I IBDV nowadays. Type II antibodies do not confer protection against type I infection, neither do they interfere with the response to type I vaccine.

The high mutation rate of the RNA polymerase of IBDV leads to antigenic variation (that is, variant strains) and modification in virulence in vivo (for example, the very virulent strain). These may require special vaccines for maximum protection. Cross protection studies have shown that inactivated vaccines prepared from “classical” type I virus require a high antigenic content to provide good protection against some of these variants.

Moreover, very virulent strains of IBDV have also emerged and caused serious disease in many countries over the past decade.

IBDV consists of two segments, designated segment A and segment B, of double stranded RNA shown by polyacrylamide gel electrophoresis. The larger segment A is approximately 3.4 kb containing two open reading frames (ORF). The larger ORF is monocistronic and encodes a polyprotein which would later be auto-processed into structural protein VP2 (40 kDa-45 kDa), VP3 (30 kDa-32 kDa) and protease VP4 (28 kDa) of IBDV (Muller & Becht, 1982 J. Virol. 1982 Oct. 44(1):384-392; Azad, A. A. et al., 1985, Virol 143: 35-44; Azad A. A. at al., 1987, Virol 161: 145-152; Hudson, P. J., et al, 1986, Nucleic Acids Res 14: 5001-5012; Kibenge F S, et al., 1997, Arch Virol 142:2401-2419). Meanwhile, the shorter, partially VP2 overlapping ORF encodes VP5 protein (17 kDa). The smaller segment B (approximately 218 kb) encodes the 90 kDa functional protein VP1 (Muller H, Nitschke R. 1987, Virology 159:174-177; Speis et al., 1987, Virus Res 8: 127-140). The genome of IBDV contains two restriction sites of VP2, namely Accl and Spel. Overlapping occurs between VP5 and VP2. VPI functions as both RNA-dependent RNA polymerase and capping enzyme for in vivo replication of the virus. It presents as a free polypeptide and as a genome-linked protein (Muller H, Nitschke R. 1987, Virology 159:174-177; Kibenge F S, Dhama V. 1997. Arch. Virol. 142:1227-1236).

Sequences of VP2 in various strains are highly conserved, except the central Accl-SpeI restriction fragment which is designated as the hypervariable region (Bayliss et al., 1990, J. Gen Virol 71, 1303-1312). The sequences that are important for the virulence and attenuation of the virus were also identifed in this region (Yamaguchi T, et al., 1996, Virology 223:219-223). VP2 is the major host-protective immunogen of IBDV that contains the antigenic sites responsible for the induction of neutralizing antibodies (Azad et al., 1987, Virol 161: 145-152) while VP3 protein is recognized by non-neutralizing antibodies.

VP2 and VP3 form the capsid of virus. VP2 is likely to be exposed on the outer surface of the capsid while VP3 is laid inside, interacting with the viral RNA.

VP4 protease is a non-structural polypeptide. It is responsible for cleavage of the polyprotein on segment A, but it is not included in the mature virion. The presence of serine-lysine catalytic dyad accounts for its proteolytic activity (Birghan et al., 2000, EMBO Journal 4: 114-123).

VP5 is not essential for viral replication in cell culture but it has a regulatory function and could play a key role in virus release and dissemination (Mundt et al., 1997, J. Virol 71:5647-51). Another disease that affects livestock is porcine reproductive and respiratory syndrome (PRRS) is caused by porcine reproductive and respiratory syndrome virus (PRRSV). Porcine Reproductive and Respiratory Syndrome (PRRS) is considered to be the most economically important viral disease of intensive swine farms in Europe and North America. The syndrome first began causing swine herd problems in the late 1980's in Unites States and prior to isolation of the causative agent, was often referred to as mystery swine disease. But in the time since the virus was identified in Europe (Leiystad Virus [LV]) in 1991 (Wensvoort G., et al., 1991, Veterinary Quarterly 13, 121-130) and in the United States (VR-2332) in 1992 (Benfield D. A., et al., 1992, J Vet Diagn Invest 4, 127-133). PRRSV has become a significant pathogen of swine herds worldwide, with new disease phenotypes continuing to emerge.

PRRS can result in losses in neonates and nurseries from respiratory disease and reproductive losses in breeding stock. As a consequence, it causes dramatic financial consequences in the swine industry. However, the inherent variability in clinical signs translates into highly variable economic losses. On a herd basis, most acute outbreaks are estimated to decrease annual production by 5%-20%.

Due to the differences in the prevailing health status of swine, virus strains and management strategies of different farms, clinical signs and production losses vary widely among herds. Moreover, many cases are often complicated with secondary infections that increase the severity of this disease (De Jong, M. F. et al., 1991, European Comm Seminar on the New Pig Disease, 4:29-30 #4; Benfield, D. A. et al., 1992, J Vet Diagn Invest. 4:127-133).

There are three phases in the acute form of PRRSV infection: the initial, climax and final phases (Raymakers, J. M. L., 1991, European Comm Seminar on PRRS 11:4-5, Brussels, #16). In the initial phase of the disease, clinical signs such as inappetance, lethargy, depression and pyrexia can be seen in the breeding/gestating, farrowing or grow/finish areas of pig farms. During this phase, respiratory disorders, such as dyspnea and polypnea, may or may not be observed in adult pigs but these syndromes are usually prominent in younger animals. It typically lasts 1-3 weeks.

During the climax phase, premature farrowings, increased stillbirths, mummifed, and weakborn pigs, and an increase in preweaning mortality can be observed. At the same time, most of the growth in reduction and mortality is due to secondary infections. Increased secondary infections occur in growing pigs, especially in the nursery (De Jong, M. F. et al, 1991, European Comm Seminar on the New Pig Disease 4:29-30 #4; Benfield, D. A., et al., 1992, Diseases of Swine 7th Ed, Ames, IA: Iowa State University Press: 756-762). This phase typically lasts 8-12 weeks.

In the final phase, reproductive parameters return to near-normal pre-PRRS levels and there are variable respiratory diseases in nursery or grow-finish pigs. This phase may either be the prelude to chronic disease or to a return to normal pre-PRRS production level.

During chronic PRRS, a long term reduction in the number of pigs born alive can be seen (Dial, D., et al., 1990, MSD Corn Mtg. Denver: Livestock Conservation Institute, 3.6) and an extended duration of reduced farrowing rates has also been observed (Benfield, D. A., et al., 1992, Diseases of Swine 7th Ed. Ames, IA: Iowa State University Press: 756-762; Benfield, D. W., et al., 1992, J Vet Diagn Invest. 4: 127-133). Pigs in chronic PRRS herds have higher feed gain ratios in addition to slower growth. Meanwhile, the increased numbers of secondary infections are probably responsible for the continued rhinitis and pneumonia observed in chronically affected herds.

Although the mechanism of reproductive failure is still unknown, transplacental infection of porcine fetuses is common in late gestation. Moreover, semen can transmit PRRSV though no virus can be isolated from the testicles or accessory sex glands of mature boars (Oblinger, V., 1992, Pig Dis Info Centre).

Porcine reproductive and respiratory syndrome virus (PRRSV) is a member of the genus Anterivirus of Arteriviridae family (Cavanagh, D. 1997, Arch of Virol. 142: 629-633). Alveolar macrophages are the target cells of PRRSV in vivo.

PRRSV is a spherical, enveloped virus 45 nm-70 nm in size (Benfield, D. A., et al., 1992, Diseases of Swine 7th Ed. Ames, IA: Iowa State University Press: 756-762; Benfield, D. W., et al., 1992, J Vet Diagn Invest. 4: 127-133) and it contains an icosahedral nucelocapsid core of 20 nm-30 nm. The lipid bilayer that surrounds the nucleocapsid contains two major envelope components, GP5 and M, and two minor envelope components, GP2 and ON. Small surface projections are also apparent. The PRRSV has a buoyant density of 1.18 g/ml-1.19 g/ml in CsCI and 1.13 g/ml-1.14 g/ml in sucrose. Peak infectivities are greater in CsCI purifed preparations than in sucrose preparations (Benfield, D. A., et al:, 1992, Diseases of Swine 7th Ed. Ames, IA: Iowa State University Press: 756-762; Benfield, D. A., et al., 1992, J Vet Diagn Invest. 4: 127-133).

Although the infectivity titer of the virus is stable for more than four months at −70° C., it is reduced 10 times when maintained at 56° C. for 15-20 minutes or at 37° C. for 10-24 hours (Benfield, D. A., et al., 1992, Diseases of Swine 7th Ed. Ames, IA: Iowa State University Press: 756-762; Benfield, D. A., et al., 1992, J Vet Diagn Invest. 4: 127-133). Virus infectivity titers are reduced over 90% at pH levels less than 5 or greater than 7. In addition, virus replication is inactivated after treatment with chloroform or ether. PRRSV is made up of a polyadenylated, single-stranded positive sense RNA molecule of 15.1 kb (Meulenberg, J. J., et al., 1993, Virol 192: 62-72), which consists of eight open reading frames (ORFs), designated Is, Ib, 2, 3, 4, 5, 6 & 7 (Conzelmann, K. K. et al., 1993, Virol 193: 329-339; Meulenberg, et al., 1995, Virology 206:155-163). In PRRSV-infected cells, the ORFs of the virus are transcribed into 3′ nested sets of sex messenger RNAs. All six mRNAs have 3′ polyA tail and a common leader sequence obtained from the 5′ end of the genomic RNA.

ORP 1a and 1b comprise 75% of the genome at the 5′ end and code for protein with apparent replicase and RNA polymerase activities. ORFs 2 to 6 encode viral membrane-associated proteins. In addition, polypeptides encoded by ORFs 2-5 are glycosylated and have been designated GP2 (291W), GP3 (431cD), GP4 (31 kD) and GPS (251W).

GP2 is one of the minor components in the viral envelope. A portion of it is folded on itself via disulfide bonds, without forming homodimers, or heter-multimers with other viral protein (Meulenberg, J. J., et al., 1996, Virol 225: 44-51).

GP3 can provide protection for piglets against PRRSV infection in the absence of a noticeable neutralizing humoral response, as demonstrated in the North American and European strains. There are highly hydrophobic sequences at the N- and C-terminal regions of GP4. Although anti-GP5 antibodies can neutralize PRRSV infection, they are less effective than anti-GP5 antibodies (Weiland, E., at al., 1999, Vet Microbiol 66: 171-186).

GP5 that incorporated into the viral envelope contains N-linked oligosaccharides of the high mannose and complex type. GP5 is important for the infectivity of PRRSV as specific anti-GP5 antibodies can neutralize PRRSV infection of susceptible cells (Pirzadeh, B. et al., 1997, J. Gen. Virol, 78:1867-1873).

The polypeptide, designated as M protein, encoded by ORF6 (19 kD) is a non-glycosylated type III transmembrane protein. It forms disulfide-linked heterodimers with the GPS glycoprotein. In PRRSV infected cells, disulfide-linked M protein homodimers have also been observed but these were not incorporated into the virions (Mardassi, H. et al., 1996, Virol 221:98-112).

ORF 7 encodes a non-glycosylated polypeptide (151cD) that forms the nucleocapsid, designated N. N is a highly basic protein predominantly present as disufide-linked homodimer (Mardassi, H. et al., 1996, Virol 221: 98-112; Meulenberg, et al., 1995. Virology 206:155-163).

Vaccines against HCV, IBDV, PRRSV and H5N1

The vast economic importance of HC initiated various efforts to extinguish the disease. The live attenuated vaccine strains, for example the Chinese strain, which is the most extensively used vaccine, can effectively protect pigs against the disease.

Although vaccination with attenuated live virus is safe and effective, it interferes with serodiagnosis and does not discriminate between vaccinated and infected animals. Therefore, the current controls used to fight against large outbreaks of HC in Europe are based largely on quarantine restriction and slaughter policy. In order to avoid trade restrictions, the slaughter policy is mostly based on the destruction of infected and serologically positive (suspected) animals. The enormous cost of eradication programs stimulated the search for alternate methods to control the disease.

Due to the robustness of IBDV, hygienic measures alone are insufficient to control the disease. Hence, the currently favored practice to control IBD is to vaccinate the parent birds with an oil-emulsion vaccine just before laying in order to induce a high level of passive immunity in the offspring, which would protect them. The progeny are then vaccinated with a killed oil emulsion vaccine at seven days old to give a protection rate of around 85% to 90% (Wyeth P J, Chettle N J, 1990, Vet Rec 126:577-578). This may be followed by a live vaccine in the drinking water at around 2.5, 3.5 and 4.5 weeks of age although researchers have shown that this offers no extra protection (Wyeth & Chettle, 1990 Vet Rec. June 9, 126(23):577-578; Goddard, et al., 1994 Pet Rec. September 17; 135(12):273-274).

However, injected vaccines will be neutralized in the presence of high level of maternal antibodies if administered too early. Hence, serological monitoring is usually necessary to determine the optimal timing for vaccination (Van den Berg T P, et al., 1991, Avian Patho 120:133-143).

With the occurrence of variant strains (with different antigenic properties) and very virulent strains (can breakthrough even high levels of maternal antibodies), classical IBDV vaccine becomes ineffective in defeating IBD.

Current strategies for the control of PRRSV depend largely on immunization with modified-live vaccines that have inherent drawbacks. Firstly, the live vaccine has the intrinsic risk of reversion to a virulent phenotype. Secondly, it is not possible to discriminate between vaccinated and infected animals in a herd. It has been reported that a DNA vaccine against PRRSV trigger both cellular mediated immunity and humoral immunity (Kwang, I., et al., 1999, Res in Vet Sci, 67:199-201).

However, despite these advances, there still exists a need for effective and easy to produce and administer vaccines for HCV, IBDV and PRRSV and other animal diseases.

### SUMMARY OF THE INVENTION

In order to provide a clear and consistent understanding of the present invention, the following list of terms and their definitions are provided.

An animal is defined as any vertebrate or invertebrate, including, but not limited to humans, birds and fish.

An antigen is defined as a macromolecule that is capable of stimulating the production of antibodies upon introduction into a mammal or other animal including humans. As used in this application, antigen means an antigen per se, an antigenic determinant of the antigen, or a fusion protein containing the antigen or antigenic determinant sometimes referred to a native epitope.

An antigenic determinant is defined as a small chemical complex that determines the specificity of an antigen-antibody reaction. Colonization and/or virulence antigens of a pathogen contain one or more antigenic determinants.

An amino acid domain is defined as an amino acid sequence within a protein that can be associated with a particular function or sequence homology.

A colonization or virulence antigen is defined as an antigen on the surface of a pathogenic microorganism that is associated with the ability of the microorganism to colonize or invade its host. Discussion and claims may refer to colonization or virulence antigens or antigenic determinants thereof. A pathogen may contain antigens of either colonization or virulence or both and one or more DNA sequences for each or both may be transferred to a vector and used to transform a plant such that it expresses the antigen or antigens.

An immunogenic agent is defined as any antigen that is capable of causing an immune response in animals such as upon oral ingestion of plants carrying vectors that express the antigen.

A chimeric sequence or gene is defined as a DNA sequence containing at least two heterologous parts, i.e., parts derived from, or having substantial sequence homology to pre-existing DNA sequences which are not associated in their pre-existing states. The pre-existing DNA sequences may be of natural or synthetic origin.

A coding DNA sequence is defined as a DNA sequence from which the information for making a peptide molecule, mRNA or tRNA is transcribed. A DNA sequence may be a gene, combination of genes, or a gene fragment.

A foreign DNA is defined as a DNA that is exogenous to or not naturally found in the microorganisms or plants to be transformed. Such foreign DNA includes viral, prokaryotic, and eukaryotic DNA, and may be naturally occurring, chemically synthesized, cDNA, mutated, or any combination of such DNAs. The foreign DNA of this invention is derived from or has substantial sequence homology to DNA of pathogenic microorganisms and viruses, or is a synthetic gene that encodes a protein that is of similar amino acid sequence to prokaryotic genes.

A fusion protein is defined as a protein containing at least two different amino acid sequences linked in a polypeptide where the sequences were not natively expressed as a single protein. Fusion proteins are frequently the result of genetic engineering whereby DNA sequences from different genes are joined together to encode a single protein composed of amino acid sequences from the originally separate genes.

A gene is defined as a discrete chromosomal region that codes for a discrete cellular product.

A microorganism is defined as a member of one of the following classes: bacteria, fungi, protozoa, or viruses.

A plant tissue is defined as any tissue of a plant in its native state or in culture. This term includes, without limitation, whole plants, plant cells, plant organs, plant seeds, protoplasts, callus, cell cultures, and any group of plant cells organized into structural and/or functional units.

The use of this term in conjunction with, or in the absence of any specific type to plant tissue as listed above or otherwise embraced by this definition is not intended to be exclusive of any other type of plant tissue. Plants suitable for transformation according to the processes of this invention included, without limitation, monocots such as corn, wheat, barley, sorghum, rye, rice, banana, and plantains, and dicots such as potato, tomato, alfalfa, soybean, beans in general, canola, apple, pears, fruits in general, and other vegetables.

A plant transformation vector is defined as a plasmid or viral vector that is capable of transforming plant tissue such that the plant tissue contains and expresses DNA not pre-existing in the plant tissue.

A foodstuff or edible plant material is defined as any plant material that can be directly ingested by animals or humans as a nutritional source or dietary complement.

A pre-existing DNA sequence is defined as a DNA sequence that exits prior to its use, in toto or in part, in a product of method according to this invention. While such pre-existence typically reflects a natural origin, pre-existing sequences may be of synthetic or other origin.

An immune response involves the production of antibodies, which are proteins called immunoglobulins. The antibodies circulate in the bloodstream and permeate other body fluids, where they bind specifically to the type of foreign antigen that induced them. Binding by antibodies frequently inactivates viruses and bacterial toxins (such as tetanus or botulinum toxin) by blocking their ability to bind to receptors on target cells. Antibody binding also marks invading microorganisms for destruction, either by making it easier for a phagocytic cell to ingest them or by activating a system of blood proteins, collectively called complement, which kills the invaders. Cell-mediated immune responses, the second class of immune responses, involve the production of specialized cells that react with foreign antigens on the surface of other host cells. The reacting cell can kill a virus-infected host cell that has viral proteins on its surface, thereby eliminating the infected cell before the virus has replicated. In other cases the reacting cell secretes chemical signals that activate macrophages to destroy invading microorganisms.

A secretory immune response (SIR) is defined as a specific type of immune response. It involves the formation and production of secretory IgA antibodies in secretions that bathe the mucosal surfaces of human and other animals and in secretions form secretory glands. An agent that causes the formation and production of such antibodies is considered to stimulate secretory immunity or to elicit a SIR. Secretory immunity is also sometimes referred to as mucosal immunity.

A substantial sequence homology is defined as a functional and/or structural equivalence between sequences of nucleotides or amino acids. Functional and/or structural differences between sequences having substantial sequence homology are frequently de minimus.

A transgenic plant is defined as a plant that contains and expresses DNA that was not pre-existing in the plant prior to the introduction of the DNA into the plant.

Transgenic plant material is any plant matter, including, but not limited to cells, protoplasts, tissues, leaves, stems, fruit and tubers both natural and processed, containing and expressing DNA that was not pre-existing in the plant prior to the introduction of the DNA into the plant. Further, plant material includes processed derivatives thereof including, but not limited to food products, food stuffs, food supplements, extracts, concentrates, pills, lozenges, chewable compositions, powders, formulas, syrups, candies, wafers, capsules and tablets.

An edible plant material includes a plant or any material obtained from a plant that is suitable for ingestion by mammal or other animals including humans. This term is intended to include raw plant material that may be fed directly to animals or any processed plant material that is fed to animals, including humans. Materials obtained from a plant are intended to include any component of a plant that is eventually ingested by a human or other animal.

An edible vaccine refers to vaccine formulations that are administered and effective via the peroral route.

Mucoadhesive polymers refers to synthetic or natural polymers that interact with the mucus layer covering the mucosal epithelial surface and mucin constituting a major part of the mucus. Recently, a drug delivery system has been developed with the use of mucoadhesive polymers that will attach to related tissue or to the surface coating of the tissue for the targeting of various absorptive mucosa such as ocular, nasal, pulmonary, buccal, vaginal mucosa etc. This system of drug delivery is called a mucoadhesive drug delivery system. Such a drug delivery system could be used to deliver the therapeutic agent of the present invention. Naturally occurring mucoadhesive polymers include, but are not limited to, hyaluronic acid and chitosan.

A protective immune response refers to immune responses resulting from vaccine administrations that can protect an animal from death when challenged within 10 times lethal dose.

The invention provides an orally acceptable immunogenic composition comprising unpurified or partially purified recombinant immunogens expressed in a plant or in bacteria, wherein the immunogens are expressed at a level such that upon oral administration of the composition to an animal, an immunogenic response is observed. Examples of the recombinant immunogens include, but are not limited to, HCV, IBDV, IBV, ILTV, and PRRSV. In one embodiment, the recombinant immunogen is a chimeric protein. In certain embodiments, the HCV, IBDV, IBV, ILTV or PRRSV immunogens are capable of generating an immunogenic response to HCV, IBDV, IBV, ILTV or PRRSV when the immunogen interacts with a mucosal membrane. In particular aspects, the HCV, IBDV, IBV, ILTV or PRRSV immunogens are capable of binding a glycosylated molecule on the surface of a membrane of a mucosal cell. In further aspects, the HCV, IBDV, IBV, ILTV or PRRSV immunogens form a chimeric protein.

The invention also provides an orally acceptable immunogenic composition comprising unpurified or partially purified recombinant immunogens expressed in bacteria, wherein the immunogens are expressed at a level such that upon oral administration of the composition to an animal, an immunogenic response is observed. An example of the recombinant immunogen may be H5N1. In one embodiment, the recombinant immunogen is a chimeric protein. In certain embodiments, the H5N1 immunogens are capable of generating an immunogenic response to H5N1 when the immunogen interacts with a mucosal membrane. In one embodiment, the H5N1 immunogen is capable of binding a glycosylated molecule on the surface of a membrane of a mucosal cell. In another embodiment, the HCV, IBDV, IBV, ILTV or PRRSV immunogens form a chimeric protein.

The present invention also provides for an orally acceptable immunogenic composition comprising unpurified or partially purified recombinant immunogens expressed in a plant or in bacteria for use as a medicament. Examples of the recombinant immunogens include, but are not limited to, HCV, IBDV, IBV, ILTV and PRRSV.

The present invention further provides for an orally acceptable immunogenic composition comprising unpurified or partially purified recombinant immunogen expressed in a bacteria for use as a medicament. An example of the recombinant immunogen includes H5N1.

The present invention also provides uses of an orally acceptable immunogenic composition comprising unpurified or partially purified recombinant immunogens expressed in a plant or bacteria for the manufacture of a medicament for the treatment of a disease caused by the immunogen. Examples of the recombinant immunogens include, but are not limited to, HCV, IBDV, IBV, ILTV and PRRSV.

The present invention further provides uses of an orally acceptable immunogenic composition comprising unpurified or partially purified recombinant immunogen expressed in a plant or bacteria for the manufacture of a medicament for the treatment of a disease caused by the immunogen. An example of the recombinant immunogen includes H5N1.

The invention also provides an orally acceptable immunogenic composition comprising unpurified or partially purified recombinant immunogens expressed in a plant, wherein the immunogens are expressed in the plant at a level such that upon oral administration of the composition to an animal, an immunogenic response is observed. Examples of the recombinant immunogens include, but are not limited to, an immunogenic protein from a cholera virus of swine, an immunogenic protein from a porcine reproductive and respiratory syndrome virus, and an immunogenic protein from an infectious bursal disease virus.

The invention further provides vaccines comprising an immunogen or immunogens of hog cholera virus, porcine reproductive and respiratory syndrome virus or infectious bursal disease virus expressed in a plant, wherein the immunogen is capable of binding a glycosylated molecule on a surface of a membrane mucosal cell. In certain embodiments, the immunogen is a hog cholera virus immunogen: in other embodiments, the immunogen is a porcine reproductive and respiratory syndrome virus immunogen. In additional embodiments, the immunogen is an infectious bursal disease virus.

The invention also provides a plant composition comprising a viral antigen which triggers production of antibodies and which is derived from a HCV, IBDV, IBV, ILTV or PRRSV antigen and plant material, the antigen being a product produced by the method of expressing the immunogen in a transgenic plant and the plant material being in a form chosen from the group consisting of a whole plant, plant part, or a crude plant extract.

The invention further provides a transgenic plant expressing a nucleotide sequence which encodes a recombinant viral antigenic protein, such as the recombinant protein derived from a cholera virus of swine, a porcine reproductive and respiratory syndrome virus or an infectious bursal disease virus. In certain aspects, the protein is chimeric. In other aspects the plant is Arabidopsis.

The invention additionally provides a vaccine composition comprising: a recombinant viral antigenic protein, the protein produced in a plant and derived from hog cholera virus, a porcine reproductive and respiratory syndrome virus or infectious bursal disease virus; and plant material, wherein the vaccine composition is capable of eliciting an immune response upon administration to an animal.

The invention further provides a food comprising transgenic plant material capable of being ingested for its nutritional value, the transgenic plant expressing a recombinant immunogen. Examples of recombinant immunogens include, but are not limited to, immunogens derived from hog cholera virus, a porcine reproductive and respiratory syndrome virus or an infectious bursal disease virus. In certain embodiments, the plant is Arabidopsis. In other embodiments, the plant is selected from the group consisting of tomato and potato. In additional aspects, the transgenic plant material is selected from the group consisting of edible fruit, leaves, juices, roots, and seed of the plant.

The invention further provides a method for constructing a transgenic plant cell comprising: constructing a DNA vector by operably linking a DNA sequence encoding a recombinant viral antigenic protein, for example, the recombinant protein derived from a hog cholera virus, a porcine reproductive and respiratory syndrome virus or an infectious bursal disease virus to a plant-functional promoter capable of directing the expression of the DNA sequence in the plant; and transforming a plant cell with the DNA vector. In certain embodiments, the transforming comprises Arabidopsis mediated transformation.

The invention additionally provides a method for producing a pharmaceutical vaccine composition, wherein the pharmaceutical vaccine composition consists of a recombinant viral antigenic protein, comprising the steps of: constructing a DNA vector by operably linking a DNA sequence encoding the recombinant viral antigenic protein, for example, the recombinant protein derived from a swine virus which is the causative agent of cholera, a porcine reproductive and respiratory syndrome virus or an infectious bursal disease virus, to a plant-functional promoter capable of directing the expression of the DNA sequence in a plant; transforming a plant with the DNA vector; and recovering the pharmaceutical vaccine composition expressed in the plant. In certain aspects, the plant is an Arabidopsis plant.

The invention additionally provides an immunogenic composition comprising unpurified or partially purified recombinant immunogen expressed in bacteria, wherein the immunogen is expressed in the bacteria at a level such that upon administration of the composition to an animal, an immunogenic response is observed. Examples of the recombinant immunogens include, but are not limited to, immunogens derived from HCV, IBDV, IBV, ILTV PRRSV or H5N1. In one embodiment, the immunogen is a chimeric protein. In certain aspects, the HCV, IBDV, IBV, ILTV, PRRSV or H5N1 immunogen is unpurified from the bacteria. In other aspects the HCV, IBDV, IBV, ILTV, PRRSV, or H5N1 immunogen is partly purified from the bacteria. In some embodiments, the HCV, IBDV, IBV, ILTV, PRRSV, or H5N1 immunogen is a chimeric protein. In other embodiments, the administration comprises injection. In further aspects, the administration comprises oral ingestion.

The invention also provides a vaccine comprising an immunogen of hog cholera virus, porcine reproductive and respiratory syndrome virus, infectious bursal disease virus, or H5N1 expressed in bacteria. In certain embodiments, the immunogen is a hog cholera virus immunogen. In other aspects, the immunogen is a porcine reproductive and respiratory syndrome virus immunogen. In additional aspects, the immunogen is an infectious bursal disease virus. In further aspects, the immunogen is an H5N1 virus.

The invention further provides a method for producing an immunogenic response to an immunogen in an animal, comprising the steps of expressing a recombinant immunogen in bacteria, wherein the immunogen is expressed in the bacteria at a level such that upon administration of the bacteria to an animal, an immunogenic response to the immunogen is observed. In certain aspects, the immunogen is derived from HCV, IBDV, IBV, ILTV, PRRSV, or H5N1. In other aspects, the immunogen is partly-purified from the bacteria.

The development of safe and efficient avian influenza vaccines for human and animal use is essential for preventing virulent outbreaks and pandemics worldwide. In the present invention, genetically modified lactic acid bacteria such as Lactococcus lactis strains expressing the avian influenza HA gene are presented as oral vaccines for the protection of H5N1 virus infection.

Several influenza H5N1 hemagglutinin (HA) antigen expression vectors were constructed based on a well-engineered nisinA-induced L. lactis expression strain. They either expressed the antigen in the cytoplasm (L. lactis-pNZ8150-HA), or secreted the antigens (L. lactis-pNZ8110-HA), or displayed the antigens on the cell wall (L. lactis-pNZ8110-pgsA-HA1). In one embodiment, these vectors were formulated with mucoadhesive polymers and surface enteric coating. After oral administration of the enteric-coated antigen displayed expression vector and antigen secreted expression vector respectively, the resultant immune responses were greatly improved, resulting in complete protection of the immunized mice from a lethal dose of viral challenge.

In one embodiment, oral administration of genetically modified Lactococcus lactis strains disclosed herein induced strong HA-specific humoral and mucosal immune responses in subjects which were able to withstand lethal doses of H5N1 virus infection.

In a further embodiment, the nisinA-induced L. lactis expression strain vector is a construct engineered to contain a plasmid expressing the pgsA gene from B. subtilis and the sequence encoding the globular domain of HA, that is, the HA1 protein. In certain embodiments, the HA1 protein is expressed on the surface of the bacteria via cell wall anchor.

The foregoing has outlined rather broadly the features and advantages of the present invention in order that the detailed description of the invention that follows may be better understood. Additional features and advantages of the invention will be described hereinafter which form the subject of the claims of the invention.

### BRIEF DESCRIPTION OF THE FIGURES

FIG. 1A shows a schematic for the construction of plasmid pNZ8110-HA. The pNZ8110-HA plasmid was created by cloning the HA gene into the pNZ8110 expression vector at the Nae1/HindIII sites so that the ssUSP sequence of empty expression vector plasmid pNZ8110 was included. FIG. 1B shows the restriction map of the recombinant plasmid construct pNZ8110-HA.

FIG. 2A shows a schematic for the construction of plasmid pNZ8150-HA. The pNZ8150-HA plasmid was created by cloning the HA gene into the pNZ8110 expression vector at the Nae1/HindIII sites so that the ssUSP sequence was included. FIG. 2B shows the restriction map of the recombinant plasmid construct pNZ8150-HA.

FIG. 3A shows a schematic for the construction of plasmid pNZ8110-PgsA-HA1. The pNZ8110-PgsA-HA1 plasmid was created by creating a fusion of a fragment containing the pgsA gene with a HA1 DNA fragment through a bridge PCR reaction, digestion with SpeI-HndIII and insertion into similarly digested pNZ8110. FIG. 3B shows the restriction map of the recombinant plasmid construct pNZ8110-PgsA-HA1.

FIG. 4 shows an agarose electrophoresis gel where Lane 1, Lane 2, and Lane 3 show, respectively, DNA marker DL15,000, pNZ8110 before being cut by double restriction endonuclease, and pNZ8110 after double restriction endonuclease cutting.

FIG. 5 shows a plasmid restriction map for the plasmid NZ9700(HA) from clone NZ9700(HA), where a 1704 bp HA gene fragment was fused into the secretion expression vector plasmid pNZ8110.

FIG. 6 shows growth curves of L. lactis-pNZ9700(HA), L. lactis-pNZ8110 and L. lactis NZ9700 (wild type). Culture samples were taken during the growth phase from 0 to 16 h. Growth of L. lactis was measured based on the optical density (OD) at 600 nm Analysis of variance (ANOVA) indicated Significance was defined as a P value less than 0.05.

FIG. 7 shows an agarose electrophoresis gel where Lane 1 and Lane 2 show respectively, DNA marker DL2,000 and PCR product showing detection of the HA insert in plasmid clone pNZ9700(HA) after electroporation into L. lactis.

FIG. 8A shows microcapsules for oral administration. FIG. 8B shows the protective effect on L. lactis by the alginate capsules.

FIG. 9 shows the timing protocol for Regimen 1 and Regimen 2 administration of L. lactis-pNZ9700(HA) and L. lactis-pNZ8110. D(↑) denotes for dosing; S (↓) for sampling.

FIG. 10 shows HA protein expression of HA of pNZ8110-HA after transformation into L. lactis strain 9700 (now labeled clone L. lactis-pNZ9700(HA)). Specific expression of HA protein was examined by SDS-PAGE (FIG. 9A) and Western blotting (FIG. 9B).

FIG. 11 shows HA-specific immune responses after oral administration of either L. lactis-pNZ9700(HA) or L. lactis-pNZ8110 for each given test group of mice. Each group of four mice was orally administered five (regimen 1) or eight (regimen 2) times over 8 weeks with 10⁹ CFU of L. lactis-pNZ9700(HA); L. lactis-pNZ8110 was used as a negative control. Antigen-specific antibody (serum IgG) titers were determined by ELISA. Antibody titers were calculated as the inverse of the dilution (2ⁿ) of serum that corresponds to two times the background absorbance. Each data point represents the mean serum IgG titer of at least four individual animals, and is representative of two experiments; error bars, standard deviation; *p<0.0001 compared to control group; **p<0.05 compared to control group. Results are expressed as the mean value (log 2)±S.D. (n=4).

FIG. 12 shows fecal IgA detected by ELISA. IgA antibody titers were monitored at the 10^{th} week after the first immunization. * Mean values significantly different between the groups. (p<0.05).

FIG. 13 shows survival rate after challenge with H5N1 virus. BALB/c mice (six per group) were administered orally with L. lactis-pNZ9700(HA) or L. lactis-pNZ8110 and challenged with H5N1 virus. The percentages of survival rate post-challenge are shown.

FIG. 14 shows the survival ratio after challenge with H1N1 virus. BALB/c mice were administered orally with L. lactis-pNZ8110-HA or wild type L. lactis NZ9700. The percentages of survival rate post-challenge are shown.

FIG. 15 shows HA-specific serum IgG was determined by ELISA. Mice were orally immunized with 150 ul 10¹⁰ CFUs of L. lactis-pNZ8110, L. lactis-pNZ8150-HA (HA protein expressed in cytoplasm), L. lactis-pNZ8110-HA (HA protein was secreted), or L. lactis-pNZ8110-pgsA-HA1 (HA protein was displayed on the surface of cell wall), on days 0˜3, 7˜10, and 21˜24. Immune sera were taken at day 34. * and ** represent statistically significant differences relative to the PBS control (*p<0.05, ** p<0.01). Data are expressed as the mean value (log 2) ±S.D. of duplicate experiments.

FIG. 16 shows HA-specific mucosal IgA was detected by ELISA. Fecal pellets were collected on day 34. * and ** represent statistically significant differences relative to the PBS control (*p<0.05, ** p<0.01). Data are given as mean±SD of duplicate experiments.

FIG. 17 shows western blot analysis of recombinant L. lacti vectors L. lactis-pNZ8110, L. lactis-pNZ8150-HA, L. lactis-pNZ8110-HA, and L. lactis-pNZ8110-pgsA-HA1 (A, B and C), and immunofluorescence analysis of L. lactis-pNZ8110-pgsA-HA1 (D) (X200).

FIG. 18 shows HA-specific antibody titers detected by ELISA. Mice (18A and 18B) were orally immunized with enteric capsules and non-enteric live bacteria. FIG. 18A shows that HA-specific serum IgG determined by ABS-ELISA using recombinant HA protein as a coating antigen. FIG. 18B showsHA-specific mucosal IgA determined from the fecal pellets.

FIG. 18C shows HA-specific IgG titers of chickens immunized orally or subcutaneously with L. lactis-pNZ8110-pgsA-HA1. Chickens sera IgG were assayed by ABS-ELISA. *Represents statistically significant differences relative to the PBS, L. lactis-pNZ8110 and capsule-L. lactis-pNZ8110 controls. (*p<0.05). Data are given as mean±SD of duplicate experiments.

FIG. 19 shows cell-mediated immune responses induced by enteric coated recombinant L. lactis. * Represents statistically significant differences relative to the PBS, L. lactis-pNZ8110 and capsule-L. lactis-pNZ8110 controls. Data are represented as mean±SD of triplicate experiments.

FIG. 20 shows immune protection against H5N1 virus lethal challenges after oral deliveries of different vaccine preparations. Mice were infected intranasally with H5N1′ virus 2 weeks after the last immunization. FIG. 20A shows mean weight loss as a percentage of body weight of mice 6 days after infection. FIG. 20B shows percent survival of mice 0-14 days after infection. n=5 mice per group.

FIG. 21 shows a plasmid construct comprising a 2189-bp fragment containing the pgsA gene and HA1 gene transcriptionally fused with pNZ8110 to generate pNZ8110-pgsA-HA1.

FIG. 22 shows a 1% agarose gel electrophoresis after SpeI-HindIII double digestion; Lane 1 is a DNA marker (500-15000 bp); Lane 2 is the pNZ8150-pgsA-HA1 plasmid construct digested with SpeI-HindIII; Lane 3 is pNZ8110 digested with SpeI-HindIII.

FIG. 23 shows a Western blot of a cell wall fraction, demonstrating that the HA1 protein is displayed on the surface of L. lactis-pNZ8110-pgsA-HA1 bacteria. Lane M is the pre-staining marker; Lane 1 is L. lactis-pNZ8110-pgsA-HA1; Lane 2 is L. lactis-pgsA. An ˜82 KDa protein band corresponding to the HA1-anchor protein while the protein band was not shown in L. lactis-pgsA.

FIG. 24 shows FACS analysis of recombinant L. lactis-pNZ8110-pgsA-HA1 cells. For each experiment, 10,000 cells were analyzed.

FIG. 25 shows immunofluorescence analysis of L. lactis-pgsA (on the left) and L. lactis-pNZ8110-pgsA-HA1 (on the right) under a fluorescent microscope (200 power).

FIG. 26 shows anti-H5N1 HA serum IgG antibody levels, as measured by ABS-ELISA using H5N1 HA recombinant protein, from mice immunized with control, L. lactis bacterial vectors, or L. lactis bacterial vector and CTB adjuvant. Serum was collected from mice orally immunized with PBS, L. lactis-pgsA, L. lactis-pNZ8110-pgsA-HA1 μL. lactis-pgsA (CTB), and L. lactis-pNZ8110-pgsA-HA1 (CTB). Data are given as mean±SD of duplicate experiments.

FIG. 27 shows anti-HGN1 HA mucosal IgA antibody levels, as measured by ABS-ELISA using H5N1 HA recombinant protein, from fecal pellets of mice immunized with control, L. lactis bacterial vectors, or L. lactis bacterial vector and CTB adjuvant. Data are given as mean±SD of duplicate experiments.

FIG. 28 shows quantification of HA-specific IFN-γ-secreting lymphocytes as detected by ELISpot assay. The lymphocytes were extracted from mice inoculated with the bacterial vaccine preparations shown along the x-axis. Data are given as mean±SD of triplicate experiments.

FIG. 29 shows percent survival of mice inoculated with different vaccine preparations 14 days after H5N1 virus lethal infection challenge. The bacterial vaccine preparations are identified in the figure legend.

### DETAILED DESCRIPTION OF THE INVENTION

This invention provides an orally acceptable immunogenic composition comprising unpurified or partially purified recombinant HCV, IBDV, IBV, ILTV or PRRSV immunogen expressed in a plant, wherein said immunogen is expressed in the plant at a level such that upon oral administration of said composition to an animal, an immunogenic response is observed.

This invention provides the above orally acceptable immunogenic composition wherein said HCV, IBDV, IBV, ILTV or PRRSV immunogen is capable of generating an immunogenic response to HCV, IBDV, IBV, ILTV or PRRSV when the immunogen interacts with a mucosal membrane.

This invention also provides the above orally acceptable immunogenic composition wherein the HCV, IBDV, IBV, ILTV or PRRSV immunogen is capable of binding a glycosylated molecule on the surface of a membrane of a mucosal cell. In an embodiment of the above invention, the said HCV, IBDV, IBV, ILTV or PRRSV immunogen is a chimeric protein.

This invention provides an orally acceptable immunogenic composition comprising unpurified or partially purified recombinant immunogens expressed in a plant, wherein said immunogens are expressed in the plant at a level such that upon oral administration of said composition to an animal, an immunogenic response is observed, said viral immunogens being an immunogenic protein or proteins from a cholera virus of swine.

This invention provides an orally acceptable immunogenic composition comprising unpurified or partially purified recombinant immunogens expressed in a plant, wherein said immunogens are expressed in the plant at a level such that upon oral administration of said composition to an animal, an-immunogenic response is observed, said viral immunogens being an immunogenic protein or proteins from a porcine reproductive and respiratory syndrome virus.

This invention provides an orally acceptable immunogenic composition comprising unpurified or partially purified recombinant immunogens expressed in a plant, wherein said immunogens are expressed in the plant at a level such that upon oral administration of said composition to an animal, an immunogenic response is observed, said viral immunogens being an immunogenic protein or proteins from an infectious bursal disease virus.

This invention provides a vaccine comprising an immunogen of hog cholera virus, porcine reproductive and respiratory syndrome virus or infectious bursal disease virus expressed in a plant, wherein said immunogen is capable of binding a glycosylated molecule on a surface of a membrane mucosal cell. In an embodiment of the above vaccine, the said immunogen is a hog cholera virus immunogen. In another embodiment, the said immunogen is a porcine reproductive and respiratory syndrome virus immunogen. In a separate embodiment, the said immunogen is an infectious bursal disease virus immunogen.

The invention also provides a plant composition comprising a viral antigen which triggers production of antibodies and which is derived from a HCV, IBDV, IBV, ILTV or RSV antigen and plant material; said antigen being a product produced by the method of expressing said immunogen in a transgenic plant, said plant material being in a form chosen from the group consisting of a whole plant, plant part, or a crude plant extract.

This invention also provides a transgenic plant expressing a nucleotide sequence which encodes a recombinant viral antigenic protein, said recombinant protein derived from a cholera virus of swine, a porcine reproductive and respiratory syndrome virus or an infectious bursal disease virus. In an embodiment, the said protein is chimeric. In another embodiment, the said plant is Arabidopsis.

This invention provides a vaccine composition comprising: a recombinant viral antigenic protein, said protein produced in a plant and derived from hog cholera virus, a porcine reproductive and respiratory syndrome virus or infectious bursal disease virus; and plant material, wherein said vaccine composition is capable of eliciting an immune response upon administration to an animal.

This invention provides a food comprising transgenic plant material capable of being ingested for its nutritional value, said transgenic plant expressing a recombinant immunogen derived from hog cholera virus, a porcine reproductive and respiratory syndrome virus or an infectious bursal disease virus. In an embodiment, the said plant is Arabidopsis.

This invention provides a food comprising transgenic plant material capable of being ingested for its nutritional value, said transgenic plant expressing a recombinant immunogen derived from hog cholera virus, a porcine reproductive and respiratory syndrome virus or an infectious bursal disease virus. In an embodiment, the said immunogen is derived from hog cholera virus. In another embodiment, the said immunogen is derived from a porcine reproductive and respiratory syndrome virus. In a separate embodiment, the said immunogen is derived from hog cholera virus. In a separate embodiment, the said plant is selected from the group consisting of: tomato and potato. In another embodiment, the said transgenic plant material is selected from the group consisting of: edible fruit, leaves, juices, roots, and seed of said plant.

This invention provides a method for constructing a transgenic plant cell comprising: constructing a DNA vector by operably linking a DNA sequence encoding a recombinant viral antigenic protein, said recombinant protein derived from a hog cholera virus, a porcine reproductive and respiratory syndrome virus or an infectious bursal disease virus to a plant-functional promoter capable of directing the expression of said DNA sequence in said plant; and transforming a plant cell with said DNA vector. In an embodiment, the said transforming comprises Arabidopsis mediated transformation.

This invention provides a method for producing a pharmaceutical vaccine composition, wherein the pharmaceutical vaccine composition consists of a recombinant viral antigenic protein, comprising the steps of constructing a DNA vector by operably linking a DNA sequence encoding said recombinant viral antigenic protein, said recombinant protein derived from a swine virus which is the causative agent of cholera, a porcine reproductive and respiratory syndrome virus or an infectious bursal disease virus, to a plant-functional promoter capable of directing the expression of said DNA sequence in a plant; transforming a plant with said DNA vector; and recovering said pharmaceutical vaccine composition expressed in said plant. In an embodiment, the said plant is an Arabidopsis plant.

This invention provides an immunogenic composition comprising unpurified or partially purified recombinant HCV, IBDV, IBV, ILTV, PRRSV, or H5N1 immunogen expressed in a bacteria, wherein said immunogen is expressed in the bacteria at a level such that upon administration of said composition to an animal, an immunogenic response is observed. In an embodiment, the said HCV, IBDV, IBV, ILTV, PRRSV, or H5N1 immunogen is unpurifed from said bacteria.

In a separate embodiment, the HCV, IBDV, IBV, ILTV, PRRSV, or H5N1 immunogen is partly purifed from said bacteria. In another embodiment, the said HCV, IBDV, IBV, ILTV, PRRSV, or H5N1 immunogen is a chimeric protein. In another separate embodiment, the said administration comprises injection. In still another embodiment, the said administration comprises oral ingestion.

This invention provides for a vaccine comprising an immunogen of hog cholera virus, porcine reproductive and respiratory syndrome virus, infectious bursal disease virus, or H5N1 virus expressed in a bacteria. In an embodiment, the said immunogen is a hog cholera virus immunogen. In a separate embodiment, the said immunogen is a porcine reproductive and respiratory syndrome virus immunogen. In another embodiment, the said immunogen is an infectious bursal disease virus. In another embodiment, the said immunogen is a an H5N1 virus.

This invention provides a method for producing an immunogenic response to an immunogen in an animal, comprising the steps of expressing a recombinant immunogen in a bacteria, wherein said immunogen is expressed in the bacteria at a level such that upon administration of said bacteria to an animal, an immunogenic response to said immunogen is observed. In an embodiment, the said immunogen is derived from HCV, IBDV, IBV, ILTV, PRRSV, or H5N1. In another embodiment, the said immunogen is partly-purified from said bacteria.

The present invention provides novel DNA vaccines and edible vaccines for veterinary infectious diseases. Traditionally, vaccines comprise attenuated and/or killed virus, sub-unit vaccine (protein based), and/or DNA vaccine (DNA based) compositions. In preferred embodiments, the present invention discloses DNA and sub-unit vaccine compositions, methods of preparation and administration. In another embodiment, disclosed herein are plant-based (e.g., edible) vaccine compositions, methods of preparation and administration, including oral vaccination.

Specifically disclosed herein are DNA and plant based vaccines and methods of preparing and administering such vaccines that combine efficacy, safety, and the opportunity for serological discrimination between vaccinated and infected animals.

Moreover, the vaccines and methods of using vaccines disclosed herein can elicit neutralizing antibodies that are regarded as an important specific defense against diseases including, but not limited to, HC, IBDV, PRRSV, and H5N1. The DNA vaccines disclosed herein further provides the advantages of chemical stability, as well as the ability to elicit both humoral and cell-mediated immunity. It is contemplated that certain embodiments of the present vaccine invention may provide better protection value than other vaccines against the same or like diseases. It has been demonstrated that using pcDNA3.1 inserted with VP5-5.2 and VP2-3.4 of IBDV HK46 was effective in fighting against IBDV.

As would be known to one of ordinary skill in the art, foreign antigenic agents for a vaccine may be produced using standard molecular biological techniques. For instance, in a well known use of this type of methodology, foreign DNA for human interferon was spliced into a plasmid vector, introduced into a bacterial cell, and then cloned. The gene for human interferon was excised from a human chromosome, and a bacterial plasmid linearized, through the use of the same restriction enzyme. The interferon was gene joined with the plasmid by sticky end ligation, and the plasmid containing the interferon gene taken up by a bacterium. Each daughter bacterium inherited the interferon gene, the interferon-producing bacterial population was grown and the interferon isolated from the bacteria for administration to an animal, specifically a human patient.

Often, such methods are clinically applied to produce bacterial antigens for components of vaccines. Note that these antigens are generally located at the surface of the bacteria or are secreted molecules. Such antigens include, but are not limited to, one or more virulence molecules, secreted proteins, processed surface proteins, outer membrane proteins, capsular antigens, toxins, pili, and/or flagella antigens.

Traditional DNA vaccines or “naked DNA” requires purification to separate the recombinant plasmid from the host cells (i.e., bacterial cells) to obtain the clinical grade quality of the DNA vaccine. This type of vaccine is generally administered by injection. The utility of naked DNA for mucosal vaccination may be limited by the lability of DNA in tissue fluids. Hence, it is likely that a barrier must be placed between DNA vaccines and extracellular digestive enzymes if effective mucosal delivery is to be achieved. In the presence of cationic lipids, transfection of various cell types (e.g., cells of stomach and colon) with DNA vaccine was facilitated via a non-specific mechanism or a physiologic pathway present (Schmid, R. M., et al., 1994, Gastroenterol, 32:665-670).

Disclosed herein, however, a DNA vaccine was complexed to E. coli. In a preferred aspect of a DNA vaccine, bacteria that are expressing a foreign immunogen expressed from a DNA vector is administered as a vaccine. Such a vaccine composition is known herein as a “DNA-Bac” vaccine. The DNA-Bac embodiment of the present invention may provide an advantage in simplicity of creation and utilization, relative to other specific types of vaccines (e.g, plant vaccines, recombinant DNA vaccines, purified protein based vaccines, live or attenuated antigen vaccines, etc.). Such a DNA-Bac vaccine of the present invention may provide the advantage of a reduced cost of vaccine production by lacking part or all of traditional purification processes, and thus be more cost-effective for use in the prevention or treatment of veterinary infectious diseases. Specifically demonstrated herein in a non-limiting embodiment, transformed bacterial host cells comprising a recombinant plasmid expressing an immunogen against a pig infectious virus (i.e., HCV) was used as a vaccine without a further purification process. The vaccine induced both humoral and cellular immunity responses. In certain embodiments, the DNA vaccine (e.g., DNA-Bac vaccine) targeted mucosal inductive sites.

In one aspect of the present invention, cultured bacteria may be prepared containing and expressing one or more genes for one or more foreign antigen(s) on one or more commercially available expression vectors, including but not limited to, plasmid, cosmid, BAC, PAC and/or P1 DNA vectors. The vector may be separated from the bacteria using methods and materials commonly utilized by those of skill in the art, including but not limited to, the use of pilot-scale plasmid preps, ultrapure 100 columns, contract CAN manufacture, large-scale plasmid preparations, EndoFree Plasmid Kits, QIAfilter Plasmid Kits, QIAGEN® Plasmid Kits, Large-scale BAC/PAC/PI/cosmid preparations, QIAGEN® Large-Construct Kit, High-throughput plasmid minipreps, QIAwell Plasmid Kits and the like. For example, bacteria containing the expression vector may be pelleted through centrifugation, undergo alkaline lysis and endotoxin removal steps, the vector further purified using, for example, a commercially available QIAGEN anion-exchange chromatography apparatus, as well as isopropanol precipitation to produce ultrapure plasmid DNA. Such methods can produce, for example, up to 100 mg high-copy plasmid DNA from 20 liters of LB culture ('60 g bacterial pellet).

An example of one type of DNA vaccine used beta-Gal as reporter construct. The DNA-Bac is active both in vitro and in vivo. A DNA-Bac vaccine against the hog cholera virus induced significant enhancement in serum antibody responses and cytotoxic T lymphocyte responses as compared with naked DNA. Specifically, neutralization titers against the hog cholera virus were compared using DNA-Bac low dose, DNA-Bac high dose, a DNA vaccine and commercial attenuated vaccine, and demonstrated the effectiveness of DNA-Bac vaccine preparations at both low and high dose administrations. A rabbit fever response assay was also conducted comparing the fever reductive ability of DNA-Bac low dose, DNA-Bac high dose, a DNA vaccine, a commercial vaccine and a control, and the efficacy of the DNA-Bac composition administered at low dose and high dose were demonstrated.

Another aspect of the present invention is a novel formulation of DNA vaccine for veterinary infection diseases. Advantages for a DNA vaccine composition, method of preparation or method of administration disclosed herein include relatively low cost and flexible routes of delivery. This is of particular usefulness in the developing world. For example, the Hong Kong and China veterinary vaccine market for chicken and pig farms are regionally focused, as most of the animal infectious viruses are geographically localized, and vaccines manufactured by foreign firms made based on foreign strains do not give full protection most of the time.

For example, since HCV and PRRSV are two devastating viral diseases of swine, they have already caused enormous financial losses worldwide. HC has already caused significant mortality and morbidity in commercial piggeries in many countries in Europe and Asia. PRRSV is now recognized throughout North America and Europe. To protect pigs against these two diseases, every individual pig has to be subjected to two sets of vaccination schemes. Such vaccination schemes suffer from the disadvantages of being time-consuming and/or money-consuming. In order to help overcome these disadvantages, and provide maximum protection against the disease, alternative vaccination methods and compositions are disclosed herein.

Additionally, the vaccines of the present invention may be used in protection for birds. For example, infectious bursal disease (IBD) is a worldwide economically important disease to poultry industry. Infectious bursal disease virus (IBDV) has been shown to be the causative agent and it is an avian lymphotropic virus that causes immunosuppression.

Thus, in certain aspects of the present invention, a DNA-Bac vaccine is contemplated for use in animals (including but not limited to pigs and chickens), for diseases including but not limited to IBD, HC and/or PRRS. It is contemplated that in certain embodiments, the invention comprises a DNA vaccine against PRRS, and thereby having use in the pig industry in protecting pigs against that disease.

Since the site and method of delivery will affect the nature of immune response (Feltquate, D. M., et al., 1997, J. Immunol, 158:2278.2284; Tones, C. A. T., et al., 1997, J Virol, 158:4529-4532), an appropriate procedure must be chosen that balances the practical aspects of vaccine delivery to large animals, with the desire to generate the most protective response possible. Intramuscular injection, although popular in many animal models, may be undesirable for livestock because of the potential effects on meat quality of food producing animals. Moreover, it is undesirable to have needle tracks or vaccine residues in the relevant tissue. Delivery of plasmid into the epithelium (skin or mucosal surfaces) is considered to be the most promising site of plasmid delivery because of the immune competence of these tissues. These tissues have highly developed immune surveillance function. Finally, because these tissues are the sites of entry by most pathogens, immunization at these sites is expected to be more effective in fighting against the disease.

Intramuscular injection was thought to lead to the rapid movement of DNA or DNA-transfected cells out of the injected muscle, so that the immune stimulatory events leading to antibodies production and cytotoxic T lymphocytes reactivity took place primarily in distal tissue.

Bone marrow derived antigen presenting cells, probably dendritic cells, have been demonstrated to be required for processing antigen expressed from DNA plasmids (Ulmer, J. B., et al., 1996, Immunol, 89: 59-67; Ulmer, J. B., el at., 1996 Cur Opin Immunol, 8:531-536), though it is not clear whether the antigen presenting cells are themselves transfected with the DNA or pick up antigen from other cells.

Although muscle contains relatively few resident dendritic cells, macrophages or lymphocytes, the recent discovery at IL-15 and its high levels of expression in skeletal muscle cells (Grabstein, K. H., et al., 1994, Science, 264:961-965) indicated that muscle cells may not be as immunologically inert as once thought. Therefore, it was also a suitable site for immunization with DNA vaccine.

Currently, DNA vaccines are delivered by intramuscular or subcutaneous injection, which can induce systematic responses, but generally no mucosal immunity. At the same time, the mucosal surface of the gastrointestinal tract is the frequent site of transmission of numerous diseases. Hence, the mucosal immune response plays an important role in the protection against viral infection. In other embodiments, it is contemplated that a DNA vaccine of the present invention (e.g., a DNA-Bac vaccine) may be orally administered.

Specifically disclosed herein are DNA vaccines, including but not limited to, peDNA3.1-VP5-5.2 & VP2-3.4 and pHCV2.5, for use in vaccination against, but not limited to, IBDV HK45 strain and HCV Alford strain, respectively. The efficiency of these two vaccines delivered to the mucosal surface of gastrointestinal tracts was demonstrated.

There are gut-associated lymphoid tissues (GALT), which consist of the organized lymphoid tissues along the gastrointestinal tract. It includes isolated lymphoid follicles, Peyer's patches, appendix, tonsils, and mesenteric lymph nodes. In addition, many small lymphoid follicles populate the mucosa. From the mouth to the anus, these follicles are covered by specialized surface epithelial cells called M cells. They play a key role in delivering samples of foreign antigens by trans-epithelial transport from the lumen to organized lymphoid tissues. It is contemplated that GALT sites on mucosal surfaces may be a desirable location for contact with the vaccines of the present invention during or after administration.

Disclosed herein, the viral coat proteins were inserted into a mammalian expression vector, pcDNA3.1 and used as the DNA vaccine. The DNA vaccine, pcDNA3.1-VP5-5 2&VP2-3.4, pHCV2.5 and pcDNA3.1-ORFS have been reported to be effective in fighting against IBD, HC and PRRS respectively when they were administered through intramuscular injection. Since intramuscular injection was undesirable for livestock because of the potential effects on meat quality of food producing animals, the effectiveness of these three DNA vaccines delivered to the mucosal surface of gastrointestinal tracts of the animals was determined.

It has been reported that combined DNA immunization could induce double-specific protective immunity and non-specific response in Rainbow Trout (Pierre et al., 1998, Virol 249: 297-306). However, the inventors know of no report of combined DNA vaccine against pig diseases. The efficacy of vaccine compositions and methods of delivery against HCV, IBDV and/or PRRSV, along or in combination, is disclosed herein.

DNA vaccines using pHCV2.5 and pcDNA3.1-ORFS constructs have been shown to be effective in protecting pigs against HC and PRRS respectively. However, exposing test subjects to two separated vaccination schemes was very time-consuming and money-consuming. Disclosed herein, the efficacy of combined DNA vaccine against HC and PRRS was demonstrated.

After ELISA and Western blot analysis, it was observed that IBD and HC DNA vaccines were both effective in triggering specific antibodies against the respective virus when they were delivered orally.

It could also be seen that combined HC-PRRS DNA vaccine was effective in triggering specific antibodies against HCV and PRRSV simultaneously. The antibody reaction was rather similar in quality and intensity to that obtained with separate immunization.

In one embodiment, the present invention provides for an edible capsule form of live, non-persisting, recombinant Lactococcus lactis (L. lactis) vaccine. In one embodiment, the vaccine is produced against the highly virulent influenza virus.

In one embodiment, the enteric-coated polymer capsule formulation produces a great improvement on the overall immunogenicities of the vaccines, resulting in complete protection and survival of mice injected with an otherwise lethal dosage of H5N1 virus.

The uniqueness of the present invention is the successful creation of an edible vaccine against an antigen such as influenza virus (H5N1) using the non-persisting L. lactis(Kimoto, H. et al., 2003, Can. J. Microbiol. 49:707-711) as the carrier, loading them on mucoadhesive polymers and packaging them in enteric-coated mini-capsules. It is demonstrated that although the viability of L. lactis is rapidly diminished in the gastrointestinal tract, the antigens they carried and produced shortly after they were administered and protected temporarily by the encapsulation were sufficient to induce significant mucosal and systemic immune responses and allow all the immunized mice to survive lethal challenge of H5N1 injection.

It is possible that the vector systems themselves had specific immune stimulation effects. It has been shown that LAB can initiate inflammatory responses and activate monocytes and other antigen presenting cells (Mercenier, A., Pavan, S. & Pot, B., 2003, Curr. Pharm. Des. 9:175-191). In the present invention, different immune responses resulted from similar vector systems that differed only in antigen expression designs that were observed. The vector in which the antigens were anchored on the surface of cell wall through a PgsA domain gave the best response in every aspect. The data therefore supports the study by Poo et al. who developed the PgsA fusion system based on the N-terminal transmembrane region of the PgsBCA enzyme complex of B. subtilis (Poo, H., et al., 2006, Int. J. Caner 119, 1702-1709). For avian influenza strains, the analysis of sera from mice supports the use of a neutralizing titer of ≧80 as an efficacy endpoint (Eichelberger, M., et al., 2007, Vaccine 26, 4299-4303). In the present invention, the neutralization titer of capsule-L. lactis-pNZ8150-HA was 80 and the survival rate was 40% after H5N1 infection. In contrast, neutralization titers of capsule-L. lactis-pNZ8110-HA and capsule-L. lactis-pNZ8110-pgsA-HA1 were 148 and 167, respectively, and these capsules provided 100% protection against H5N1 virus challenge. Similar plans for H5N1 challenges in chickens are in progress pending regulatory approvals.

The influenza HA antigen gene cloned in the vectors is from A/chicken/Henan/12/2004(H5N1). L. lactis-pNZ 8150-HA and L. lactis-pNZ8110-HA contained the full length HA gene, while L. lactis-pNZ8110-pgsA-HA1 contained the HA1 portion of the HA. They all induced significant humoral and systemic immune responses. Previous studies have suggested HA1 should contain almost all the HA epitopes (Jeffery, K. & Taubenberger, J. K., 1998, Proc. Natl. Acad. Sci. USA 95:9713-9715; Shih, A. C. C. et al. (2007) Proc. Natl. Acad. Sci. USA 104:6283-6288; Suzuki, Y. et al., 2000, J. Virol. 74:11825-11831). However, since HA1 is more prone to mutations and antigenic changes (Stevens, J., et al., 2004, Science 303, 1866-1870), it is important to design the live vector vaccines to be quickly adaptive to accommodate virus evolutions. The nisinA-induced recombinant L. lactis antigen expression vectors used herein are very flexible in design and can be optimized for antigen expression and presentation. It is also a well-engineered stable system for large scale production (de Ruyter, P. G. et al., 1996, Appl. Environ. Microbiol. 62:3662-3667; Bahey-El-Din, M., et al., 2008, Vaccine 26, 5304-5318). The polymer mini-capsule formulation developed was also easy and inexpensive to manufacture. Although it is a simple design, the improvement in vaccine efficacy was highly significant.

In one embodiment, the present invention provides for a method of using genetically modified lactic acid bacteria such as Lactococcus lactis strains expressing the avian influenza HA gene as an oral vaccine for protection against H5N1 virus infection. In one embodiment, the oral administration of recombinant L. lactis NZ9700 (HA) microcapsules can induce significant HA-specific humoral and mucosal immune responses, and most importantly, provide protection against H5N1 virus challenge.

In one embodiment, the method comprises an oral dosing regimen which can be easily administered to both human and animal populations. In another embodiment, the method has the ability to generate a mucosal immune response.

The present invention provides a method of inducing immune responses to an antigen, comprising the step of administering to an animal or human genetically modified lactic acid bacteria expressing the antigen. Examples of lactic acid bacteria include, but are not limited to, Lactococcus, Streptococcus, Lactobacillus, Leuconostoc, Pediococcus, Brevibacterium and Propionibacterium. In one embodiment, the lactic acid bacteria are of the genus Lactococcus as described in U.S. Pat. Nos. 5,580,787, 6,333,188, and 7,553,956. In another embodiment, the lactic acid bacteria are of the species Lactococcus lactis.

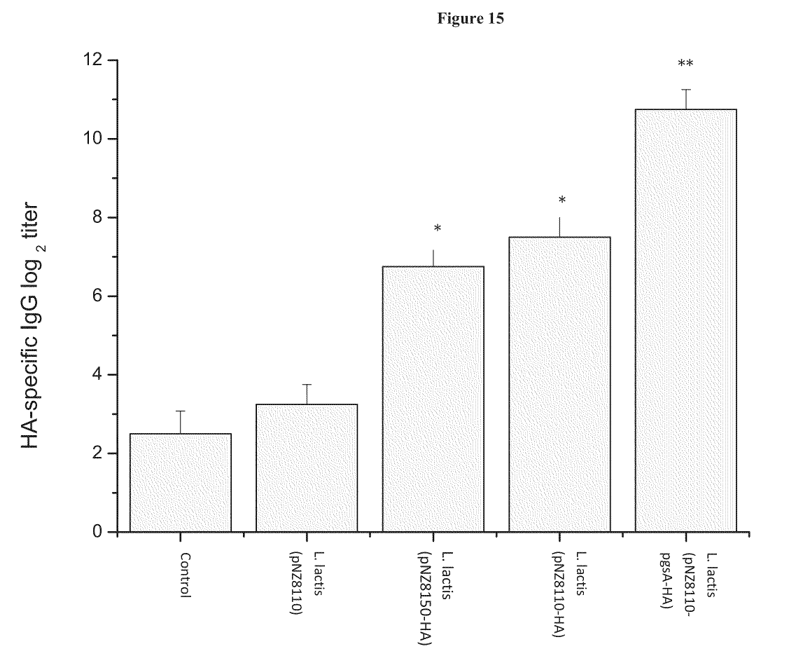

The genetically modified lactic acid bacteria of the present invention are capable of inducing immune responses when administered to a subject. Immune responses induced by the bacteria of the present invention include, but are not limited to, humoral immune responses, cellular immune responses, and mucosal immune responses. For example, the bacteria of the present invention are capable of inducing systemic IgG responses and mucosal IgA responses. In another embodiment, the bacteria of the present invention are capable of inducing cellular immune responses (see e.g. FIG. 15).

In one embodiment, the genetically modified lactic acid bacteria of the present invention are capable of inducing protective immune responses, i.e. immune responses that can protect immunized subjects from lethal challenges of pathogens (such as viruses or bacteria).

In general, the lactic acid bacteria of the present invention are genetically modified to express one or more antigens. In an embodiment, said antigens are heterologous. Examples of heterologous antigens include, but are not limited to, bacterial, protozoan, fungal, and viral antigens. Sources of heterologous antigens include, but are not limited to, influenza virus, helicobacter pylori, Salmonella, rotavirus, respiratory coronavirus, etc. as described in U.S. Pat. Nos. 6,551,830 and 7,432,354.

In one embodiment, a viral antigen such as hemagglutinin of avian influenza virus H5N1 can be expressed in genetically modified lactic acid bacteria.

The genetically modified lactic acid bacteria of the present invention can be administered in amounts and by using methods that can readily be determined by persons of ordinary skill in this art. The vaccines of the present invention can be administered and formulated, for example, for oral administration, either as liquid solutions or suspensions, or solid forms suitable for solution in, or suspension in, liquid prior to administration. The preparation may also be emulsified, or the ingredients mixed with excipients such as, for example, pharmaceutical grade mannitol, lactose, starch, magnesium stearate, sodium saccharine, cellulose, magnesium carbonate, and the like. These compositions take the form of solutions, suspensions, tablets, pills, capsules, sustained release formulations, nose drops or powders.

The vaccines of the present invention can also be in the form of injectables. Suitable excipients would include, for example, saline or buffered saline (pH about 7 to about 8), or other physiologic, isotonic solutions which may also contain dextrose, glycerol or the like and combinations thereof. However, agents which disrupt or dissolve lipid membranes such as strong detergents, alcohols, and other organic solvents should be avoided. In addition, if desired, the vaccine may contain minor amounts of auxiliary substances such as wetting or emulsifying agents, pH buffering agents, and/or adjuvants well-known in the art which enhance the effectiveness of the vaccine.

Generally, the vaccine of the present invention may be administered orally, subcutaneously, intradermally, or intramuscularly in a dose effective for the production of the desired immune response. The vaccines are administered in a manner compatible with the dosage formulation, and in such amount as will be prophylactically and/or therapeutically effective. The quantity to be administered depends on the subject to be treated, the capacity of the subject's immune system to develop the desired immune response, and the degree of protection desired. Precise amounts of the vaccine to be administered in view of the subject and antigen used could be readily determined by one of skill in the art.

In one embodiment, the genetically modified lactic acid bacteria of the present invention can be administered to a subject in a number of ways, such as orally, or by intranasal administration, intramuscular injection, subcutaneous injection, and vaginal application as described in U.S. Pat. Nos. 7,541,044, and 7,476,686. In another embodiment, it can be administered to a subject through a food product such as milk, cheese, yogurt, or kefir.

The genetically modified lactic acid bacteria of the present invention can be formulated in a number of ways, such as encapsulated inside acid labile microcapsules, enteric coated microcapsules and capsules, polymer hydrogels, or adhesive polymer patches. In one embodiment, the vaccine vector can be delivered by transmucosal delivery through the use of mucoadhesive polymers. Transmucosal delivery allows rapid uptake of a therapeutic agent into systemic circulation and avoids the first pass metabolism and/or some of the body's natural defense mechanism.

The present invention also provides genetically modified lactic acid bacteria expressing a heterologous antigen. Examples of lactic acid bacteria and heterologous antigens have been described above. In one embodiment, these lactic acid bacteria can be used as oral vaccines.

It should be noted that the various parameters of the present invention can be readily adjusted or substituted by one of ordinary skill in the art. For example, one of ordinary skill in the art would readily recognize that the present invention is not limited to the lactic acid bacteria or the viral antigen hemagglutinin described above. The present invention is equally applicable to using other bacteria and/or antigens known in the art. For example, the present invention can use other probiotics which are live microorganisms thought to be healthy for the host organism. Lactic acid bacteria and bifidobacteria are the most common types of microbes used as probiotics; but certain yeasts and bacilli generally known in the art are also useful. Examples of useful bacteria include, but are not limited to, Lactobacillus bulgaricus, Streptococcus thermophilus, Lactobacillus bifidus, Lactobacillus acidophilus, Lactobacillus casei, Bifidobacterium animalis, Bifidobacterium breve, Bifidobacterium infantis, Bifidobacterium longum, Escherichia coli, Lactobacillus paracasei, Lactobacillus johnsonii, Lactobacillus plantarum, Lactobacillus reuteri, Lactobacillus rhamnosus, Saccharomyces boulardii.

Moreover, the parameters of the number and size of the capsules used, as well as the species of the mucoadhesive polymers and enteric coating employed can be readily determined and adjusted through standard experimentation. For example, any or all of these parameters can be determined or adjusted by standard in vitro or in vivo titration experiments, using various biological responses or animal survival as experimental readouts.

In one embodiment, the present invention provides a composition for inducing immune responses to an antigen, the composition comprises genetically modified bacteria that express the antigen, wherein the bacteria are formulated with mucoadhesive polymers. In one embodiment, the bacteria are lactic acid bacteria such as Lactococcus lactis. In general, the antigen can be a bacterial antigen or a viral antigen. For example, the viral antigen is hemagglutinin of avian influenza virus H5N1. In one embodiment, the mucoadhesive polymers employed are hydrophilic polymers or hydrogels. Examples of hydrophilic polymers include, but are not limited to, poly vinyl pyrrolidone, methyl cellulose, sodium carboxy methylcellulose, and hydroxy propyl cellulose. In one embodiment, the bacteria are formulated in enteric coated solid dosage forms.

The present invention also provides a method of inducing immune responses to an antigen, comprising the step of administering to a subject (such as a human or a non-human animal) the composition described above. In general, the induced immune responses include humoral immune responses, mucosal immune responses, cellular immune responses, or protective immune responses. In one embodiment, the composition is administered orally. In another embodiment, the composition is formulated in enteric coated solid dosage forms.

The present invention also provides uses of the genetically modified bacteria described herein as a medicament for inducing immune responses in a subject. In one embodiment, the medicament is administered orally. In general, the medicament can be applied for uses in subject such as human, animal, fish, bird, and veterinary uses.

The present invention also provides a genetically modified lactic acid bacteria expressing a heterologous antigen, wherein the bacteria are formulated with mucoadhesive polymers. Examples of mucoadhesive polymers have been described above. In one embodiment, the lactic acid bacteria are of the genus Lactococcus. In another embodiment, the lactic acid bacteria are of the species Lactococcus lactis. In general, the heterologous antigen can be a bacterial antigen or a viral antigen. In one embodiment, the heterologous antigen is hemagglutinin of avian influenza virus H5N1. In another embodiment, the present invention also provides a composition comprising the above-described genetically modified lactic acid bacteria that express a heterologous antigen.

The present invention also provides a kit for inducing immune responses in a subject, wherein the kit comprises the genetically modified bacteria as described herein.

The present invention provides for a method of using genetically modified lactic acid bacteria such as Lactococcus lactis strains expressing the avian influenza HA gene as an oral vaccine for protection against H5N1 virus infection. In one embodiment, the oral administration of recombinant L. lactis NZ9700 (HA) microcapsules can induce significant HA-specific humoral and mucosal immune responses, and most importantly, provide protection against H5N1 virus challenge.

In one embodiment, the method comprises an oral dosing regimen which can be easily administered to both human and animal populations. In another embodiment, the method has the ability to generate a mucosal immune response.

The genetically modified lactic acid bacteria of the present invention are capable of inducing immune responses when administered to a subject. Immune responses induced by the bacteria of the present invention include, but are not limited to, humoral immune responses and mucosal immune responses. For example, the bacteria of the present invention are capable of inducing systemic IgG responses and mucosal IgA responses.

In one embodiment, the genetically modified lactic acid bacteria of the present invention are capable of inducing protective immune responses, i.e. immune responses that can protect immunized subjects from lethal challenges by pathogens (such as viruses or bacteria).

In general, the lactic acid bacteria of the present invention are genetically modified to express one or more antigens. In an embodiment, said antigens are heterologous. Examples of heterologous antigens include, but are not limited to, bacterial, protozoan, fungal, and viral antigens. Sources of heterologous antigens include, but are not limited to, influenza virus, helicobacter pylori, Salmonella, rotavirus, respiratory coronavirus, etc. as described in U.S. Pat. Nos. 6,551,830 and 7,432,354.

In one embodiment, a viral antigen such as hemagglutinin of avian influenza virus H5N1 can be expressed in genetically modified lactic acid bacteria.

The genetically modified lactic acid bacteria of the present invention can be administered in amounts and by using methods that can readily be determined by persons of ordinary skill in this art. The vaccines of the present invention can be administered and formulated, for example, for oral administration, either as liquid solutions or suspensions, or solid forms suitable for solution in, or suspension in, liquid prior to administration. The preparation may also be emulsified, or the ingredients mixed with excipients such as, for example, pharmaceutical grade mannitol, lactose, starch, magnesium stearate, sodium saccharine, cellulose, magnesium carbonate, and the like. These compositions take the form of solutions, suspensions, tablets, pills, capsules, sustained release formulations, nose drops or powders.

The vaccines of the present invention can also be in the form of injectables. Suitable excipients would include, for example, saline or buffered saline (pH about 7 to about 8), or other physiologic, isotonic solutions which may also contain dextrose, glycerol or the like and combinations thereof. However, agents which disrupt or dissolve lipid membranes such as strong detergents, alcohols, and other organic solvents should be avoided. In addition, if desired, the vaccine may contain minor amounts of auxiliary substances such as wetting or emulsifying agents, pH buffering agents, and/or adjuvants well-known in the art which enhance the effectiveness of the vaccine.

Generally, the vaccine of the present invention may be administered orally, subcutaneously, intradermally, or intramuscularly in a dose effective for the production of the desired immune response. The vaccines are administered in a manner compatible with the dosage formulation, and in such amount as will be prophylactically and/or therapeutically effective. The quantity to be administered depends on the subject to be treated, the capacity of the subject's immune system to develop the desired immune response, and the degree of protection desired. Precise amounts of the vaccine to be administered in view of the subject and antigen used would be readily determined by one of skill in the art.

In one embodiment, the genetically modified lactic acid bacteria of the present invention can be administered to a subject in a number of ways, such as orally, or by intranasal administration, intramuscular injection, subcutaneous injection, and vaginal application as described in U.S. Pat. Nos. 7,541,044, and 7,476,686.

The genetically modified lactic acid bacteria of the present invention can be formulated in a number of ways, such as encapsulated inside acid labile microcapsules, enteric coated microcapsules and capsules, polymer hydrogels, or adhesive polymer patches. The term enteric coating or enteric coated refers to a barrier applied to oral medication that controls the location in the digestive system where it is absorbed. Enteric coatings prevent the release of medication before it reaches the small intestine. Enteric coatings work because they are selectively insoluble substances—they will not dissolve in the acidic juices of the stomach, but they will dissolve when they reach the higher pH of the small intestine. Materials used for enteric coatings include, but are not limited to: fatty acids, waxes, shellac, plastics, and plant fibers. In one embodiment, compositions of enteric coating include, but are not limited to, cellulose acetate phthalate (CAP), methyl cellulose phthalate, hydroxylpropyl methyl cellulose acetate succinate (hypromellose acetate succinate), polyvinyl acetate phthalate (PVAP), methyl methacrylate-methacrylic acid copolymers, sodium alginate and stearic acid. One of ordinary skill in the art would readily formulate the bacteria of the present invention with any of the known enteric coating materials and methods.

Drug delivery using mucoadhesive dosage forms via transmucosal routes bypasses hepato-gastrointestinal first pass elimination associated with oral administration, thereby increasing the bioavailability and producing longer therapeutic effects. In general, there are two broad classes of mucoadhesive polymers: hydrophilic polymers and hydrogels. Hydrophilic polymers containing carboxylic groups exhibit very good mucoadhesive properties; representative examples include, but are not limited to, poly vinyl pyrrolidone (PVP), methyl cellulose (MC), sodium carboxy methylcellulose (SCMC), hydroxy propyl cellulose (HPC) and other cellulose derivatives. Hydrogels are the class of polymeric biomaterials that swell by absorbing water through adhesion with the mucus that covers epithelia. In general, hydrogels are a class of polymer material that can absorb large amounts of water without dissolving due to physical or chemical crosslinkage of hydrophilic polymer chains. One of ordinary skill in the art would readily construct hydrogels from various well-known monomers, prepolymers or existing hydrophilic polymers. Polymeric biomaterials useful for hydrogel formations usually possess anionic groups (e.g. polyacrylates and their crosslinked modifications, etc) and/or cationic groups (such as chitosan and its derivatives).

For bioadhesion or mucoadhesion to occur, a succession of phenomena is required. The first stage involves an intimate contact between a mucoadhesive and a membrane, either from a good wetting of the mucoadhesive surface, or from the swelling of the mucoadhesive. In the second stage, after contact is established, penetration of the mucoadhesive into the crevices of the tissue surface or inter penetration of the chains of the mucoadhesive with those of the mucus take place.

On a molecular level, mucoadhesion can be explained based on molecular interactions. The interaction between two molecules is composed of attraction and repulsion. Attractive interactions arise from Van der waals forces, electrostatic attractions, hydrogen bonding and hydrophobic interactions. Repulsive interactions occur because of electrostatic and steric repulsion. For mucoadhesion to occur, the attractive interaction should be larger than non-specific repulsion.

Factors affecting mucoadhesion include:

1. Polymer related factors:
i) molecular weight; ii) concentration of active polymer; iii) flexibility of polymer chains; iv) spatial confirmation; and v) swelling.

2. Environment related factors:
i) pH of polymer—substrate interface; ii) applied strength; and iii) initial contact time.

3. Physiological factors:
i) mucin turns over and ii) disease state.

Systems used for the delivery of therapeutic agents via the oral route must be designed conscious of the physiology of the gastrointestinal tract. The anatomy and physiology of route of administration dictate many of the requirements for the delivery. For example, the delivery system must be able to withstand the saliva, as saliva contains digestive enzymes and other reagents for breaking down whatever is placed in the mouth. The stomach, the main digestive organ of the body, contains many digestive enzymes and has very low pH. The pH of the stomach has been measured from 1.4 to 2.1. This harsh environment causes the destruction and denaturation of proteins. The pH of the stomach changes to 4 when food is present.

Once through the harsh conditions of the stomach the delivery system reaches the small intestine, which is divided into three regions. The first region, closest to the stomach, is the duodenum, followed by the jejunum and ileum. The further down the small intestine the fewer nutrients are taken into the bloodstream. The duodenum, about 10 inches in length, composes the first 5% and the jejunum the following 40% of the length of the small intestine. The entire length of the small intestine is 5 meter and residence time within the organ typically ranges from 2-4 hr.

The lining of the small intestine is composed of the serous, muscular, areolar, and mucous layers. Only the mucous and areolar layers are the important layers with respect to drug delivery. Transport of nutrients into the body occurs through the mucous cell layer and the areolar layer where the nutrients are taken into the blood stream.

Based on the above considerations, one of ordinary skill in the art would readily devise a delivery system useful for the present invention. The present invention encompasses synthetic or naturally occurring mucoadhesive polymers that interact with the mucin and/or mucus layer covering the mucosal epithelial surface. It also encompasses the next generation mucoadhesive polymers. For example, there has been an increasing interest from researchers in targeting regions of the gastrointestinal tract using more selective molecules capable of distinguishing between the types of cells found in different areas of the gastrointestinal tract. This concept is specifically based on certain materials that can reversibly bind to cell surfaces in the gastrointestinal tract. These next generation of mucoadhesives function with greater specificity because they are based on receptor-ligand-like interactions in which the molecules bind strongly and rapidly onto the mucosal cell surface directly rather than the mucus itself. One such class of molecules that fulfill these unique requirements is lectins.

The present invention also provides genetically modified lactic acid bacteria expressing a heterologous antigen. Examples of lactic acid bacteria and heterologous antigens have been described above. In one embodiment, these lactic acid bacteria can be used as oral vaccines.

The present invention also provides a composition comprising the genetically modified lactic acid bacteria described herein. In one embodiment, the composition further comprises a pharmaceutically acceptable carrier.

The present invention also provides uses of the genetically modified lactic acid bacteria described herein as a medicament for inducing immune responses in a subject.

In another aspect of the present invention, one or more selected pathogen's genes are introduced into plants and the transgenic plants induced to manufacture the encoded antigens (e.g., proteins).

In one embodiment, the plant is edible (e.g., a potato plant) and the vaccine administered by consumption. Edible vaccine embodiments possess the advantages of being inexpensive (i.e., it is generally less costly to grow plants than bacteria based vaccines, and generally less expensive to produce than other vaccine production methods that incorporate various purification steps), easy to administer (e.g, the plant vaccine added to animal feed), lacking injection-related hazards and eliminating or reducing the risk of contamination of animal pathogens.

There are four generally used genetic expression transformation systems that can be used for producing transgenic plants capable of being administered as one of the active agents in oral vaccines against a desired antigen. The present invention takes advantage of all four of these expression systems of the construction of edible vaccines. It should be recognized that this list is not meant to exhaust other possible approaches. The list is simply included to provide a proper context for the scope and teaching of the present invention.

First, in transgenic plants, expression vectors including the CaMV 35S promoter and antigen coding sequences can be used to constitutively transform the plants where expression in the leaves allows for rapid analysis of gene expression and biochemical characterization of gene products.

In plants such as but not limited to Brassiea napus (canola), expression vectors including the 2S albumin promoter and antigen coding sequences can be used to cause seed-specific gene expression to create the production of recombinant protein in seed tissues, routinely used as animal feed, providing for the production of attractive oral immunogenicity analyses.

In plants such as but not limited to Solanum tuberosum (potato), expression vectors including the patatin promoter or soybean vspB promoter and antigen coding sequences can be used to cause tuber-specific gene expression to create tuber-specific production of recombinant protein in tuber tissues routinely used as food. This provides for the production of attractive oral immunogenicity analyses.

Finally, in plants such as but not limited to Musa acuminala (banana), expression vectors including fruit ripening-specific promoters and antigen coding sequences can be used to transform plants that produce the recombinant protein in ripened fruit where production of recombinant protein is produced directly as candidate vaccines for ingestion studies in animals and humans.

The retention of biological properties in the recombinant proteins produced in plants, specifically ligand binding and the presentation of antigenic epitopes, is of considerable importance to the successful production of edible vaccines in transgenic plants. The ultimate test of the value of proteins of pharmacological importance is their biological activity. Vaccines are of particular interest for studies of protein expression since their effects can be accurately quantified in animal models. In addition, relatively low amounts are required, since their effects are amplified by the immune system.

Disclosed herein, in a non-limiting example, are compositions and methods for the expression of HCV antigen(s) in transgenic plant(s) that act as an edible vaccine against Hog Cholera Virus. In a demonstration of an aspect of the present invention, local strains of a pathogen (e.g., HCV) were isolated and characterized. In a specific aspect of the present invention, the E₂ region was used in designing the constructs (e.g, pHCV 1.25 and pHCV2.5). In a particular aspect of the disclosed invention, the codon usage of HCV was changed in transgenic plants. It is contemplated that expression of one or more immunogens of one or more pathogens may likewise be enhanced by changes in codon usage when expressed in plants.

In certain aspects, it is contemplated that the vaccine inventions disclosed herein may be used alone or in combination with one or more other pharmacological or therapeutic agents. In certain aspects, such an agent may comprise a biopharmaceutical, including but not limited to, one or more cytokines including but not limited to one or more interferons, interleukins, colony stimulating factors, and/or tumor necrosis factors; one or more antisense nucleic acid compositions and the like; one or more cytokines; one or more gene therapy agents or methodologies; one or monoclonal antibodies; one or more clotting factors; one or more additional vaccines or vaccine related compositions or methods of administration; and/or one or more hormones.

### A. Construction and Generation of a DNA Vaccine

The strategy of constructing and generating a DNA IBD and HC vaccine is described herein as a merely as an exemplary aspect of the present invention. However, the present invention is not limited to the specific constructs disclosed herein. As would be understood by those of ordinary skill in the art, other constructs may be created and other genes from various pathogens may be expressed using the techniques described herein to produce a DNA vaccine. In certain preferred aspects, the DNA constructs are expressed in a microorganism (e.g. bacteria). In certain aspects, a DNA/bacterial vaccine (“DNA-Bac”) is partly purified. As used herein, “partly purified” means removal of about 10%, about 20%, about 30%, about 40%, about 50%, about 60%, about 70%, about 80%, about 90%, or greater, and any range derivable therein, of bacterial component(s) from the DNA construct and/or expressed immunogen(s). In additional aspects, a DNA vaccine is administered orally, to a mucus membrane, or a combination of both. However, it is contemplated that such a DNA and/or bacteria vaccine may be administered using any technique known to those of ordinary skill in the art, and it is further contemplated that the DNA and/or bacteria vaccine may be administered in combination with one or more immunogenic or pharmacological agents, such as, for example, an adjuvant.

### 1. Design of DNA Construct

For IBD vaccine, VP5-5.2 and VP2-3.4 were cloned into pcDNA (+) vector under the control of a CMV promoter.

For HC vaccine, three consecutive genes (E₀, E₁ and E₂) that encode viral structural glycoprotein were cloned into expression vector pcDNA3,1 with CMV promoter. The recombinant plasmid was called pHCV2.5 since the size of the insert was about 2.5 kb. For PRRS vaccine, ORFS of PRRSV that encodes the major envelope protein GP5 was cloned into expression vector peDNA3.1 with upstream CMV promoter. This vector was designed specifically for eukaryotic expression. These three constructs have been prepared and their identities have been confirmed previously.

2. Transformation of E. coli Cell

501 thawed competent E. coli cells and 5 l plasmid DNA were added to a cuvette that has been chilled on ice for 5 minutes. It was then placed into the sample chamber of a bacteria electroporator (Bio Rad E. coli Pulser Transformation Apparatus), which has been set to 1.8 kV. 1 ml SOC medium (Gibco BRL) was added immediately to the cuvette after applying the pulse. After 1 hour of inoculation at 37° C., 100 l of transformed cells was spread on a LB agar plate containing ampicillin and it was incubated overnight.

### 3. Large-Scale Plasmid Extraction for Injection

A single transformed colony on the agar plate was picked and inoculated in 2 ml LB supplemented with ampicillin. It was grown at 37° C. overnight with shaking. 1.5 ml culture was then added to 1.5 L LB supplemented with ampicillin and grown at 37° C. overnight with shaking. The cells were spun down at 8000×g (Beckman JA-14 Rotor) for 10 minutes and the supernatant was discarded. The cells were resuspended in 75 ml P1 solution (50 mM glucose, 25 mM Tris, pH 8, 10 mM EDTA). 102.5 ml P2 solution (0.2 M NaOH, 1% sodium dodecyl sulfate) was added and it was allowed to stand at room temperature for 5 minutes. 150 ml P3 solution (5 M potassium acetate solution, pH 4.8) was then added. After standing on ice for 30 minutes, the mixture was centrifuged at 8000 rpm for 10 minutes (Beckman JA-14 Rotor). 375 ml isopropanol was added to the supernatant and allowed to stand at 4° C. overnight. It was then centrifuged at 12000 rpm for 10 minutes (Beckman JA-14 Rotor). The pellet was resuspended in 112.5 ml water containing RNase A. 7.5 ml P3 solution and 112.5 ml 100% ethanol was then added and it was kept at −20° C. for 1 hour. After centrifuging at 12000 rpm for 10 minutes (Beckman JA-14 Rotor), the pellet was washed with 150 ml 70% ethanol. It was centrifuged again at 12000 rpm for 10 minutes (Beckman JA-14 Rotor). The pellet obtained was then resuspended in 2 ml 1×PBS and its concentration measured by fluorometer.

### Large-Scale Bacteria Preparation for Feeding

A single transformed colony containing the desired plasmid on the agar plate was inoculated in 2 ml LB supplemented with ampicillin. It was grown at 37° C. overnight with shaking. 1.5 ml culture was then added to 1.5 L LB supplemented with ampicillin and grown at 37° C. overnight with shaking.

The cells were spun down at 8000×g for 10 minutes (Beckman JA-14 Roster) and the supernatant was discarded. The E. coli cells were dried at 37° C. overnight and were resuspended in 1×PBS. The resuspension was then sonicated (Branson Sonifer 250) for 10 minutes. 1% v/v antibiotics (Penicillin-Streptomycin, Gibco BRC) were finally added to it.

### Construction and Generation of a Plant Vaccine

The construction and generation of transgenic A. thaliana II that expresses the HCV structural genes is described herein as a merely as an exemplary aspect of the present invention.

However, the present invention is not limited to a specific species of plant and genes derived from pathogens. As would be understood by those of ordinary skill in the art, other plants may be transformed and other genes from various pathogens may be expressed using the techniques described herein to produce a plant based, and preferably edible plant vaccine, of the present invention. In certain aspects, an immunogen expressed in a plant is partly purified. As used herein, “partly purified” means removal of about 10%, about 20%, about 30%, about 40%, about 50%, about 60%, about 70%, about 80%, about 90%, or greater, and any range derivable therein, of plant component(s) from the DNA construct and/or expressed immunogen(s). In additional aspects, the vaccine is administered orally, to a mucus membrane, or a combination of both. However, it is contemplated that such a plant or edible vaccine may be administered using any technique known to those of ordinary skill in the art, and it is further contemplated that the plant or edible vaccine may be administered in combination with one or more immunogenic or pharmacological agents, such as, for example, an adjuvant.

### 1. Strategy

To construct and generate transgenic A. thaliana II that expresses the HCV structural genes, plasmid pBI 121 was digested with BarHI and Sst1, and the vector fragments recovered. pHCV2.5/pHCV1.25 were also digested with BamHI and Xba1 to release the HCV2.5 and HCVI.25 fragments for insertion into the BamHI and Xba1 digested pBS plasmid. This construct was then digested with BamH1 and Sst1. pHCV2.5 codon usage was altered by recombinant PCR and the modified HCV 1.25 TA cloned into pBS. BamHI and Sst1 digestion was conducted and the fragments recovered.

The recovered fragments from above and the HCV2.5/HCV 1.25 released by BamHI and Sst1 digestion described above underwent various ligation reactions to produce the pBI 121-HCV2.5, pBI 121-HCV 1.25 and pBI 121-M 1.25 vectors. These vectors were then transformed into Agrobacterium by electroporation. A. thaliana II was then transformed by floral dip, and underwent kanamycin selection for transgenic plants. The production of HCV transgenic plants was confirmed with PCR, Southern hybridization and Northern hybridization. Plants producing high-level expression were screened for using Western hybridization and ELISA. Finally, animals were inoculated using oral administration of transgenic plant material. Additional details of these methods are described in the following sections.

### 2. Plant Material

The seeds of Arabidopsis thaliana Columbia was purchased from LEHLE Company, P.O. Box 2366 Round Rock, Tex. 78680-2366 USA. Columbia (Col-0) is wild type Arabidopsis. The plants grow in a controlled chamber or green house at 22° C.-‘25° C. with 24 hours continuous light and all the treatments for plant growth follow the standard protocol (Jose M et al. 1998). They are briefly included, 2-days cold treatment (2° C.-4° C.) to break dormancy of the seeds, planting the germinated seeds in 9 cm pots covered with nylon window screen, and sub-irrigating the plants with water and nutrient solution regularly. The plants begin to flower and are ready for floral dip transgene after growing 3-4 weeks. The seeds will be harvested in other 3-4 weeks after the secondary floral dip.

3. Agrobacterium tumefaciens

Agrobacterium tumefaciens strain LBA4404 (Ooms et al., Plant Mot Biol. 1, 265-276, 1982) or BHA105 (Hood et al., Transgenic Research. 2, 208-218, 1993) were used in the project. The bacteria accommodated binary plasmid pBI121 with different DNA fragments, HCV2.5 kb, HCVI.25 kb and Modified HCV1.25 kb, encoding structural viral glycoproteins were inoculated into 4 mL of LB with kanamycin and rifampin. The tubes were shaken 36-48 hours at 25° C.-28° C., 250 rpm. The small culture was added into 2 L flask with 500 mL of the same medium. The large-scale culture was shaken for about 16 hours as the small culture did. Cells were harvested by centrifugation for 20 min at room temperature at 6,000 g and then resuspended in floral dip solution to a final OD 600 of approximately 0.8 prior to use.

### 4. Transgene and Selection

The method of plant transgene production used in the research is floral dip (Steven J. et al. 1998). It briefly includes the following steps: clip the primary inflorescences 3-5 days before transgene introduction to get more flowers from secondary bolts; then prepare about 500 mL of floral dip solution with agrobacteria fresh from 1 L of the above-referenced culture for 8-10 pots of plants. The plants were covered with dark plastic bags and kept in the dark overnight. After 15-24 hours the treated plants were put back into chamber or green house. The plants were dipped again in the floral dip solution in the next week. The seeds (T₁) were harvested and selected on 20-30 ug kanamycin/mL of half MS medium. After 10-15 days of selection the plants (T₁) with 4-6 green adult leaves and with well established roots in the selection medium were taken out of the selection medium and planted into heavily moistened potting soil. The T₂ seeds were harvested independently from each individual of T₁ plants and selected again on the same kanamycin medium. The progeny of false transgenic plants (escape from the selection) cannot tolerate the kanamycin exerted to the plants. The true transgenic T₁, however, will pass the kanamycin resistance gene to the majority of the T₂ seedlings (the ratio=3/4) and these lines would be the successfully transformed plants.

### 5. Identification of Transgenic Plants

The DNA samples for PCR were prepared from a small bit of plant leaves with the following protocol, which was successfully used in rapeseed. Small pieces of leaves (about 50 mg) were ground in EP tubes frozen with liquid N₂. After adding 50 L of extraction buffer (100 mM KCl, 50 mM Tris HCl pH 8.0, 1% PVP, 0.5% SDS, 5 mM EDTA) the tubes were boiled for 5 min. Take out the tubes and put them on ice. Centrifuge 5 min at top speed and take the supernatant for PCR. The PCR to check transgene plants was carried out in 25 L of PCR reaction mix, in which 2 L of prepared DNA was included. Primer pairs located in various regions of HCV2.5 kb, HCV1.25 kb and modified 1.25 kb (M1.25), were used to confirm the presence of HCV gene in the transgenic plants in different individuals of the possible transgenic plants, Southern and Northern hybridization were carried out by using genomic DNA and total RNA prepared from different transgenic lines of T₂ plants, which were confirmed by both secondary selection and PCR reaction. The probes were labeled with 32P-dATP and hybridized to the DNA and RNA membranes according to the protocols in Molecular Cloning (Fritsch, Maniatis and Sambrook., 1989, Molecular Cloning, A Laboratory Manual (Second Edition. pp 18.47-18.75)).

### 6. Construction of Recombinant Plasmids for Transgene Introduction

Binary plasmid pBI121 (Clontech Laboratories, Inc., Palo Alto, Calif.) was digested with BmnHI & Sst1 and the large vector fragment was recovered from agarose gel. Three different viral DNA fragments, HCV 2.5 kb, HCV 1.25 kb and modified HCV 1.25 kb, which encoded the HCV structural glycoprotein E(MS)/EVE₂, E₂ and modified E₂, respectively, were digested with the same restriction enzymes and recovered to insert them into the vector. The recombinant plasmid was transformed into the agrobacteria with electroporation. The transformants inserted with HCV gene were used in plant transgene introduction.

### 7. Construction of HCV2.5 and HCVI.25

The HCV 2.5 kb fragment inserted into pcDNA 3.1, named pHCV2.5, for expression in mammalian cells as DNA vaccine was used in the construction. The 2.5 kb DNA fragment covers the single strand RNA genome from 1118 to 3701, which encodes viral coat protein E₂, and other two structural glycoproteins E^{ms} and E₁. A large ORF encoded by −12.5 kb single strand RNA genome of HCV is translated to a large polyprotein of about 3900 amino acids, which is cleaved into individual proteins during virus assembling. Since E₂, E^{ms} and E₁ all are located in the middle of the polyprotein, the artificial start and stop codons are joined to the construct for their correct expression in plants. The construction (see the strategy above) in brief, included the following steps:

(1) Digest pHCV2,5 plasmid DNA with BamHI and XbaI and recover the 2.5 kb fragment, which has been added both artificial star and stop codons on either side.

(2) Insert the 2.5 kb DNA fragment into pBluescript SK at the same restriction enzyme sites for getting appropriate enzyme sites to clone it into plant transgene vector pBI121.

(3) Digest pBluescript-HCV2.5 with BanrH1 and SsII, recover the 2.5 kb fragment and insert it into the same enzyme digested plant expression vector pBI121.

(4) Transform the constructed pBI121-HCV2.5 into Agrobacterium LBA4404 and EHA105 after the construct was confirmed with PCR, Southern hybridization and partial sequencing.

The same procedure was used to construct the plant expression pBI121-HCV 1.25. The only difference was that the HCV 1.25 kb fragment coding E₂ viral coat protein was obtained from other clones for DNA vaccine pI-ICV1.25, and the pBI121-HCV1.25 was confirmed by PCR amplification of pBI121 HCV2.5, pBI121 HCV1.25 and pB1121 1.25 PCR products of pBI121 HCV2.5 in LA4404 and EHA10S; pB1121 HCV1.25 in LBA4404 and EHA105; and pBl 121 M1.25 in LBA4404 and EHA105. Addition confirmation was made by separate enzyme digestion of p81121 HCV2.5 and pHI12I with BamHI, Ssil+BamHI, and Pstl. Further, pBS HCV2.5 was digested with EcoRI+BamHI, and PstI. Southern analysis of pHI121HCV1.25 using HCV2,5 probe was also used for confirmation, as well as DNA sequencing (SEQ ID NO:3 and SEQ ID NO:4 for HCV2.5 and HCV 1.25, respectively).

### 8. Construction of Modified HCV1.25

The modification of the HCV 1.25 kb fragment was carried out with the strategy of cascade recombination PCR. Total 10 primers were designed, seven of which were modified primers and the rest of three were the adapted primers. 34 codons were changed, which consisted of 8.02% of 424 total codons in HCV 1.25. The modification procedure included two steps. The changes were introduced into 6 small fragments that overlapped each other by PCR with the sequence-modified primers. Then the modified small fragments were combined together by several times recombination PCR. The modified fragment was finally cloned and sequenced to confirm their changes. SEQ ID NO:5 shows the unmodified sequence of HCV1.25, and SEQ ID NO:6 shows the modified HCV1.25 sequence, designated M1.25. M1.25 has an artificial start ATG and stop TAG codons introduced on both sides of the original HCV1.25 sequence.

After modification, the codon usage of M1.25 was similar with that in plants; however there was no change of any amino acid sequence in terms of the original viral sequence. The frequencies of HCV 1.25 before and after the modification, and comparison with those of A. thalianai are displayed in Table 1.

TABLE 1
The codon usage of HCV1.25, M1.25 and A. thalianai

| | | | | scrofa | | | | | | | | |
|---|---|---|---|---|---|---|---|---|---|---|---|---|
| | | | As | dome | | | Aa | | Aa | | | |
| Species | | PIG | of | 11 | aa of | ATH | of | Arabidopsis | of | | Aa of | M1.25 |
| No. | Genes | 60 | PIG | CDS's | Sus s | 32 | ATH | 33661CDS's | AT | HCV1.25 | HCV | HCV |
| ARG | CGA | 5.8 | 55.3 | 1.2 | 28.4 | 3.6 | 51.2 | 6.4 | 53.5 | 2.2 | 43.3 | 0 |
| | CGC | 13.1 | | 1.9 | | 4.7 | | 3.7 | | 0 | | 0 |
| | CGG | 10.9 | | 1.7 | | 2.5 | | 4.8 | | 2.2 | | 2.3 |
| | CGT | 4.3 | | 1.5 | | 14 | | 8.8 | | 0 | | 0 |
| | AGA | 9.8 | | 19.9 | | 14.8 | | 19 | | 20.6 | | 21.3 |
| | AGG | 11.4 | | 2.2 | | 11.6 | | 10.9 | | 18.3 | | 18.9 |
| LEU | CTA | 5.1 | 99.1 | 1.2 | 28.1 | 6.9 | 83.5 | 10.1 | 94.5 | 25.2 | 105.2 | 18.9 |
| | CTC | 21.7 | | 5.6 | | 19.9 | | 15.7 | | 13.7 | | 16.5 |
| | CTG | 49.6 | | 11.1 | | 5.9 | | 10 | | 29.8 | | 16.5 |
| | CTT | 8.5 | | 4.4 | | 24.4 | | 24.2 | | 4.5 | | 21.3 |
| | TTA | 4 | | 2.4 | | 4.4 | | 13.2 | | 13.7 | | 14.2 |
| | TTG | 10.2 | | 3.4 | | 22 | | 21.3 | | 18.3 | | 18.9 |
| SER | TCA | 8 | 71.9 | 89.6 | 197.3 | 10.8 | 65 | 18.3 | 88.7 | 9.1 | 38.6 | 9.4 |
| | TCC | 21.4 | | 30.2 | | 12.2 | | 10.9 | | 9.1 | | 7.1 |
| | TCG | 4.2 | | 18.3 | | 4.9 | | 9 | | 0 | | 0 |
| | TCT | 11.5 | | 39 | | 17.4 | | 25 | | 2.2 | | 2.3 |
| | AGC | 18.7 | | 13.4 | | 11 | | 11.2 | | 9.1 | | 7.1 |
| | AGT | 8.1 | | 6.8 | | 8.7 | | 14.3 | | 9.1 | | 9.4 |
| THR | ACA | 11.3 | 54.2 | 50 | 116.6 | 14.8 | 57.6 | 15.9 | 51.2 | 27.5 | 89.3 | 26 |
| | ACC | 25.3 | | 32.8 | | 19.4 | | 10.1 | | 32.1 | | 33 |
| | ACG | 8.3 | | 10.4 | | 5 | | 7.6 | | 9.1 | | 9.4 |
| | ACT | 9.3 | | 23.4 | | 18.4 | | 17.6 | | 20.6 | | 21.3 |
| PRO | CCA | 11.7 | 58.7 | 13.1 | 57.5 | 16.6 | 48.5 | 16.1 | 47.8 | 6.8 | 41 | 11.8 |
| | CCC | 24.7 | | 21.8 | | 8.2 | | 5.2 | | 18.3 | | 7.1 |
| | CCG | 8.8 | | 10 | | 7.2 | | 8.2 | | 9.1 | | 11.8 |
| | CCT | 13.5 | | 12.6 | | 16.5 | | 18.3 | | 6.8 | | 11.8 |
| ALA | GCA | 12.7 | 75.2 | 72.9 | 130.1 | 14.8 | 82.3 | 17.4 | 63.5 | 16 | 50.2 | 16.5 |
| | GCC | 34 | | 23.7 | | 19.7 | | 10 | | 9.1 | | 7.1 |
| | GCG | 9.5 | | 10.7 | | 8 | | 8.6 | | 11.4 | | 9.4 |
| | GCT | 19 | | 22.8 | | 39.8 | | 27.5 | | 13.7 | | 11.8 |
| GLY | GGA | 15 | 78.1 | 86.1 | 178.3 | 34.1 | 81.7 | 23.5 | 64.2 | 16 | 84.7 | 18.9 |
| | GGC | 33.5 | | 32.2 | | 10.8 | | 8.9 | | 18.3 | | 14.2 |
| | GGG | 18.7 | | 46.1 | | 7.5 | | 10.1 | | 32.1 | | 16.5 |
| | GGT | 10.9 | | 13.9 | | 29.3 | | 21.7 | | 18.3 | | 35.5 |
| VAL | GTA | 4.5 | 60.7 | 2.7 | 73.2 | 4.3 | 71.4 | 10.2 | 67.2 | 25.2 | 93.8 | 18.9 |
| | GTC | 18.5 | | 31 | | 19.6 | | 12.5 | | 20.6 | | 18.9 |
| | GTG | 28.7 | | 33.4 | | 17.9 | | 17.3 | | 36.6 | | 35.5 |
| | GTT | 9 | | 6.1 | | 29.6 | | 27.2 | | 11.4 | | 18.9 |
| CYS | AAA | 20 | 57 | 8.2 | 16.9 | 18.6 | 56.8 | 31.3 | 63.8 | 20.6 | 55 | 21.3 |
| | AAG | 37 | | 8.7 | | 38.2 | | 32.5 | | 34.4 | | 35.5 |
| ASN | AAC | 24.4 | 38.6 | 6.6 | 13.8 | 25.3 | 36.3 | 20.7 | 43.8 | 13.7 | 32 | 14.2 |
| | AAT | 14.2 | | 7.2 | | 11 | | 23.1 | | 18.3 | | 18.9 |
| GLN | CAA | 7.8 | 39.3 | 3.2 | 27.6 | 17.4 | 36.9 | 19.7 | 34.7 | 13.7 | 22.8 | 14.2 |
| | CAG | 31.5 | | 24.4 | | 19.5 | | 15 | | 9.1 | | 7.1 |
| HIS | CAC | 13.8 | 20.3 | 4.1 | 6.8 | 11.6 | 19.4 | 8.6 | 22.7 | 18.3 | 22.8 | 16.5 |
| | CAT | 6.5 | | 2.7 | | 7.8 | | 14.1 | | 4.5 | | 7.1 |
| GLU | GAA | 22.5 | 62.5 | 27.5 | 36.5 | 29.8 | 65.1 | 35 | 67.1 | 25.2 | 57.3 | 26 |
| | GAG | 40 | | 9 | | 35.3 | | 32.1 | | 32.1 | | 33.1 |
| ASP | GAC | 29.3 | 49 | 8 | 15.5 | 25.2 | 53.1 | 17.1 | 54.3 | 27.5 | 50.4 | 30.8 |
| | GAT | 19.7 | | 7.5 | | 27.9 | | 37.2 | | 22.9 | | 21.3 |
| CYR | TAC | 20.6 | 30.5 | 5.4 | 9 | 20.7 | 28.7 | 13.5 | 28.7 | 38.9 | 50.3 | 40.2 |
| | TAT | 9.9 | | 3.6 | | 8 | | 15.2 | | 11.4 | | 11.8 |
| CYS | TGC | 17.5 | 28 | 6.4 | 12.3 | 9.8 | 17.5 | 7.2 | 18.1 | 32.1 | 34.3 | 21.3 |
| | TGT | 10.5 | | 5.9 | | 7.7 | | 10.9 | | 2.2 | | 14.2 |
| HE | TTC | 22.1 | 36 | 5.8 | 9.6 | 29.3 | 42.5 | 20.3 | 43 | 18.3 | 43.5 | 18.9 |

### 9. Plant Transgene

A total 10 batches of plants were treated with floral dip for transgene introduction at that point. About 80 transgenic plants which expressed kanamycin resistance on selected medium were moved into soil, and other T₁ or T₂ plants were selected on kanamycin medium. Some T₁ plants (growing big enough) were confirmed by PCR to prove the presence of HCV gene. All the data is summarized in Table 2.

TABLE 2
Summary of transgenic plants

| Batch | Gene & | Date of floral dip | | Selection | Kan^{r} |
|---|---|---|---|---|---|
| No. | Bacteria | (M/D/Y) | Seed | (ug/mL) | Plants |
| 1 | 2.5/105a | 09/28/00; 10/05/00 | 0.2 | 50 | No* |
| 2 | 1.25/4404b | 03/11/00; 10/11/00 | 0.2 | 50 | No |
| | 2.5/105 | as above | 0.2 | 50 | No* |
| 3 | 1.25/4404 | 12/15/00; 12/25/00 | 0.2 | 50 | No |
| 4 | M1.25/105 | 02/03/01; 02/15/01 | No** | | |
| | M1.25/4404 | as above | No | | |
| | 1.25/105 | as above | No | | |
| | 1.25/4404 | as above | No | | |
| 5 | M1.25/4404 | 03/08/01; 03/15/01 | 3.5 | 20~30 | 1 |
| 6 | 1.25/4404 | 03/16/01; 03/23/01 | 4.4 | 20 | No |
| 7 | 2.5/105 | 03/19/01; 03/26/01 | 4.4 | 20 | 42 |
| 8 | 1.25/105 | 03/31/01; 04/07/01 | 5 | 20 | 36 |
| 9 | pBI121/4404 | 04/04/01; 04/12/01 | 1.5 | 20 | no |
| 10 | 2.5/4404 | as above | 2 | 20 | no |

a—2-5 = pBI121 HCV2.50 105 = EHA105
b—1.25 = pBI121 HCV1.25, 4404 = LVA4404
*The possible transgenic seeds and plants were lost either by treatment with the wrong kanamycin concentration in the selection of T₂ or contamination with fungi.
**This batch of plants was treated for too long in floral dip at the time of first transformation resulting in the serious damage of the plants. In the second floral dip, unfortunately, there was a mistake in the sugar concentration and the plants died after the second floral dip.

From Table 2 it is shown that most of the transgenic plants were transformed with the strain JHA10S and only 1 plant was transformed with LBA4404. The transformation frequency of LBA4404 was about 1/175000, or 1/80000 if the seedlings selected with 30 g/mL of kanamycin were deducted, for these seedlings perhaps suffered too high a concentration of kanamycin and could not grow even when transformed with kanamycin resistant gene NPT 11. The strain EHA10S, however, gave an average transformation frequency of 0.02%, which was similar with the frequency reported by others. Although the frequency seems to be very low it is acceptable since about 4 gram of seeds can be obtained easily in one transgene experiment, which in turn would represent about 40 transgenic plants.

### 10. Identification of Transgenic Plants Using PCR

The kanamycin resistant plants were moved out of the selection plates and were planted into soil. When the plants grew with 8-10 leaves in chamber a small piece of leaf was taken from each plant to extract its DNA. Different pairs of primers were used to check the presence of HCV genes in the transgenic plants of HCV2.5.

The size of PCR products always changed correctly with the size predicted according to primers ZW3 and ZW5 (SEQ ID NOS:7 and 8, respectively) which were specifically designed to amplify the BAN gene from A. thaliana based on the A. thaliana BAC clone T13M11 (GenBank access number AC005882). The expected PCR product was part of the dihydroflavol 4-reductase gene, a single copy gene in A. thaliana, and these primers correctly produced a 780 base pair (bp) positive control amplification product. While HCV2.5 specific primers shown in SEQ ID NOS:9 and 10 correctly produced a 321 bp amplification product, genomic DNAs of three HCV2.5 and three HCV1.25 transgenic plants were amplified with the primers F01/1106 (SEQ ID NOS: 11 and 12) to produce a 2.5 kb fragment that includes sequences encoding the E0, E1 and E3 peptides, and F02/HQ6, (SEQ ID NOS:13 and 11), which produced intact full-length HCV2.5 and HCV1.25, respectively; and transgenic plants of M1.25 and HCV1.25 were confirmed using 2 pairs of primers corresponding with different regions of M1.25 used to confirm transgenic plant M1.25, non-transgenic plant controls, and template DNA fee controls were amplified with the primers producing about 1 kb fragment) demonstrating the DNA prepared in using the disclosed methods herein was good for PCR and confirming that there indeed were the viral DNA copies in the plant genome. Specific primers used to modify the E₂ gene included SEQ ID NOS:14-16. Specific amplification products included a 354 bp product of SEQ ID NOS:9 and 15, a 644 bp product of SEQ ID NOS:14 and 16, and a 1060 bp product of SEQ ID NOS:14 and 12. Almost all the kanamycin resistant plants gave a positive PCR result.

The present invention provides a composition for inducing an immune response to an antigen in a subject, comprising a genetically modified bacterium or plant expressing the antigen. In one embodiment, the bacterium is formulated into microcapsules. One of ordinary skill in the art would readily formulate the bacteria into microcapsules. An example of microcapsule formulation is described herein. In one embodiment, the bacterium is complexed with a DNA expressing the antigen. In one embodiment, the bacterium is of the genus Lactococcus.

In another embodiment, the composition of the present invention comprises a plant that is an edible plant, or in the form of a whole plant, plant part or plant extract. For example, the plant is Arabidopsis.

In one embodiment, the antigen expressed by the genetically modified bacterium or plant is a bacterial antigen or viral antigen. For example, the antigen is hemagglutinin of avian influenza virus H5N1. In another embodiment, the antigen is capable of binding a glycosylated molecule on the surface of a mucosal cell membrane. In yet another embodiment, the antigen is a chimeric protein.

The present invention also provides a method of inducing an immune response to an antigen in a subject, comprising the step of administering to said subject the composition described herein. In one embodiment, the immune response is a humoral immune response, mucosal immune response, or protective immune response. In another embodiment, the composition is administered orally.

The present invention also provides a composition as described herein for use as a medicament for inducing an immune response in a subject.

The present invention also provides uses of the composition described herein for the preparation of a medicament for inducing an immune response in a subject.

The invention will be better understood by reference to the Experimental Details which follow, but those skilled in the art will readily appreciate that the specific experiments detailed are only illustrative, and are not meant to limit the invention as described herein, which is defined by the claims which follow thereafter.

Throughout this application, various references or publications are cited. Disclosures of these references or publications in their entireties are hereby incorporated by reference into this application in order to more fully describe the state of the art to which this invention pertains. It is to be noted that the transitional term “comprising”, which is synonymous with “including”, “containing” or “characterized by”, is inclusive or open-ended and does not exclude additional, un-recited elements or method steps.

Throughout the examples recited below, for simplicity in nomenclature, certain plasmids are referred to without reference into the existing L. lactis bacterial strain into which they have been transformed. The recombinant L. lactis bacterial strains are either L. lactis NZ 9000 or L. lactis NZ 9700 into which the relevant plasmids have been transformed. One strain is used per example: consequently, a reference to a strain such as “L. lactis-pNZ8110-HA” may refer to either NZ 9000 or NZ 9700 transformed with pNZ8110-HA, as dictated in that particular example.

### Example 1

### Creation of Plasmid Constructs

In the present invention, various recombinant L. lactis vectors encoding the hemagglutinin (HA) gene of avian influenza virus H5N1 was constructed. Certain live vectors were encapsulated inside alginate microcapsules or enteric coating capsules to protect them from acid destruction and maintain antigen expression for an extended time period. Mice that were immunized orally mounted an effective immune response against H5N1 virus.

### Creation of Plasmid Constructs

A 1704 bp fragment containing the HA gene from pGEM-HA (kindly originally supplied by Prof. Ze Chen, Wuhan, China) was amplified by polymerase chain reaction (PCR) using the following primer pairs with NaeI or HindIII sites underlined (5′-tctgccggcgagaaaatagtgcttctt-3′ (SEQ ID NO: 53), 5′-cccaagctttaaatgcaaattctgcattgtaacg-3′ (SEQ ID NO: 54). The PCR product was sequenced. The resulting NaeI/HindIII fragment was cloned into various plasmids which were transformed into L. lactis bacterial vectors. Subsequently, the HA gene was synthesized from published gene sequences and cloned into the recited vectors in like manner.

pNZ8110 is an empty expression vector plasmid purchased from NIZO (Netherlands), comprised of plasmid pNZ8148 with a usp45 (ssUSP) secretion signal sequence. Bacteria transformed with pNZ8110 (i.e. L. lactis-pNZ8110), and the plasmid itself were used as a control in various experiments throughout this specification. The avian influenza HA gene was cloned into pNZ8110 in various different ways.

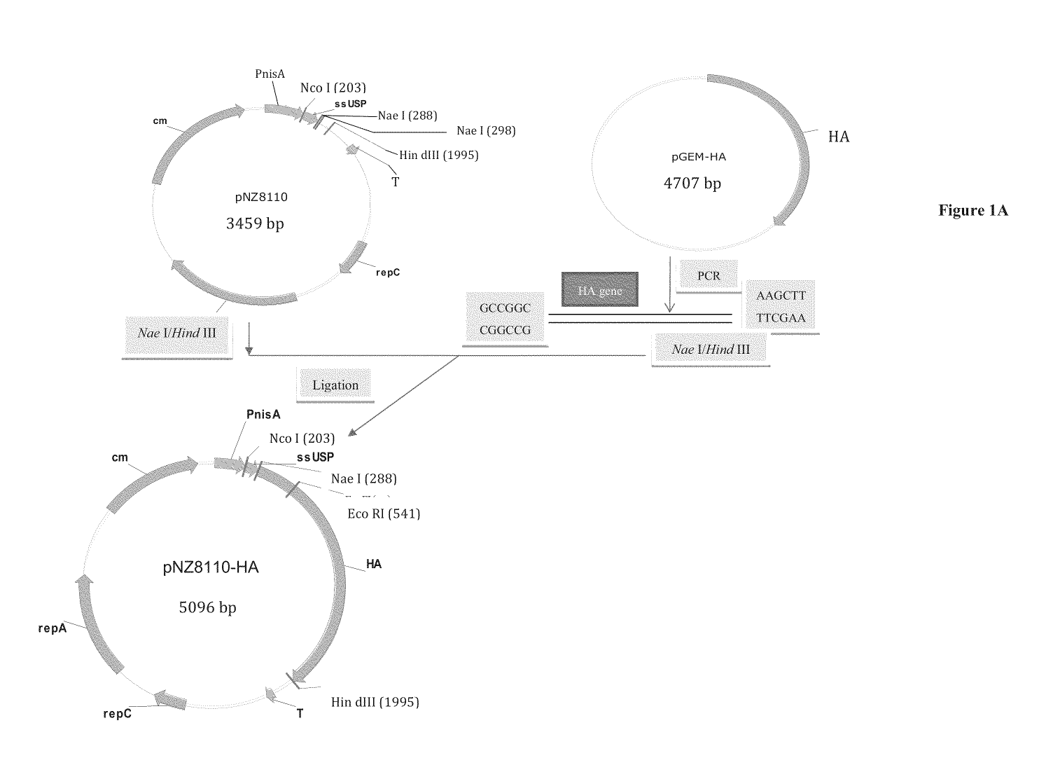

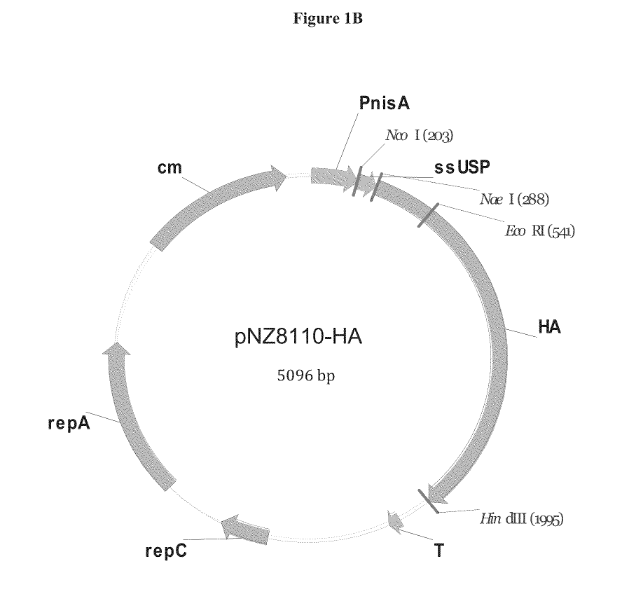

The pNZ8110-HA plasmid was created by cloning the HA gene into the pNZ8110 expression vector at the Nae1/HindIII sites so that the ssUSP sequence was included. After transformation, this resulted in an L. lactis strain in which HA protein was secreted. The creation of this construct is shown in FIG. 1A. The restriction map of the recombinant plasmid construct is shown in FIG. 1B.

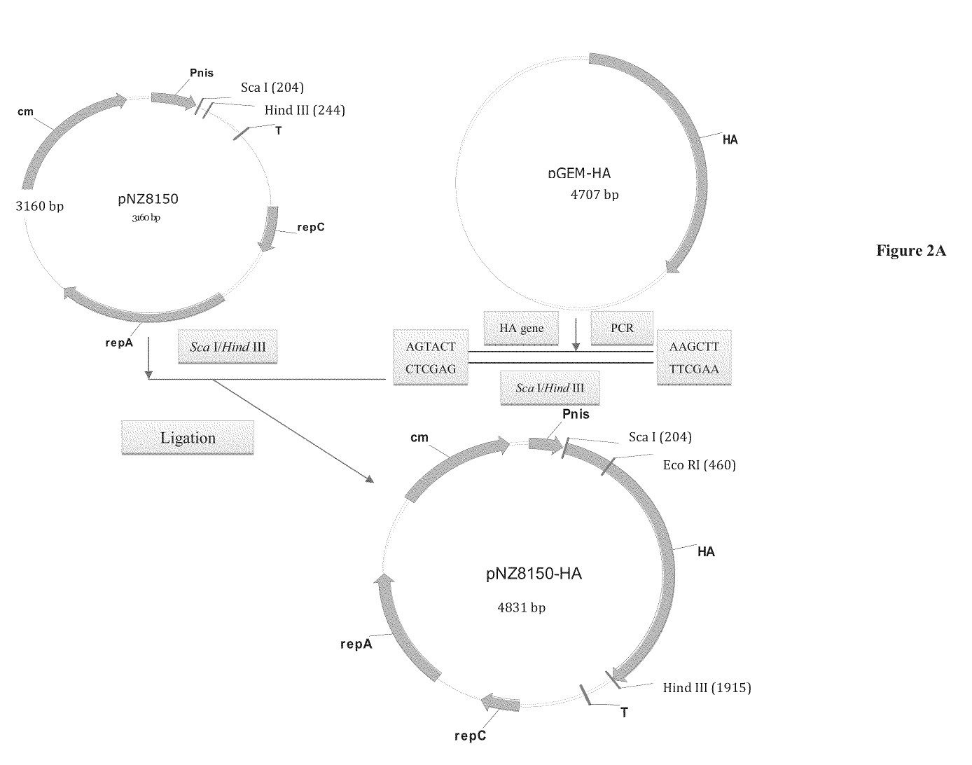

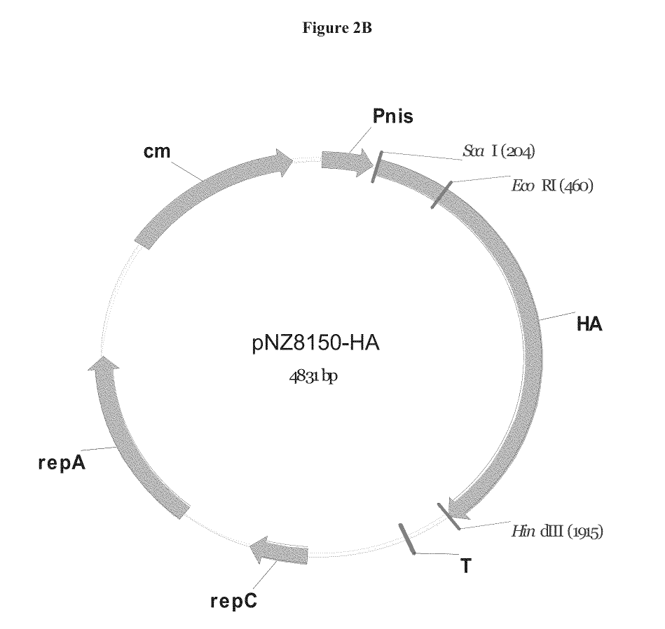

The pNZ8150-HA plasmid was constructed through the cloning of the HA gene into the pNZ8110 expression vector at the Nco1/HindIII sites so that the ssUSP sequence was not included. Consequently, after transformation, this resulted in an L. lactis strain in which HA protein was not secreted (HA protein was expressed in cytoplasm). The creation of this construct is shown in FIG. 2A. The restriction map of the recombinant plasmid construct is shown in FIG. 2B.

The pNZ8110-PgsA-HA1 plasmid was constructed. This recombinant plasmid was designed not only to include the hemagglutinin 1 (HA1) gene of influenza virus H5N1, but to include PgsA, a C-terminal anchor protein from Bacillus subtilis, designed to ensure the presence of the HA1 protein anchored on the bacterial cell wall.

In order to construct the pNZ8110-PgsA-HA1 plasmid, a 1136 bp fragment containing the pgsA gene was amplified from the chromosomal DNA of B. subtilis by polymerase chain reaction (PCR) using primers pgsA-F (5′-ggactgt atgaaaaaagaactgagctttcatg-3′ (SEQ ID NO: 55)) and pgsA-R (5′-tcctcctggggatccagattttagtttgtcactatg-3′ (SEQ ID NO: 56)), wherein the Spe I site is underlined. The 1053 bp HA1 gene fragment encoding the HA1 protein containing the sequence encoding the globular domain of HA was amplified from [A/chicken/Henan/12/2004(H5N1) [GenBank accession number: AY950232] using the following primer pairs with Knd III sites underlined (HA1-F: 5′-ggatccccaggaggagatcagatttgcattggt-3′ (SEQ ID NO: 57), HA1-R: 5′-cccaascttttatctcttttttc-3′ (SEQ ID NO: 58)). Then the pgsA DNA fragment and HA1 DNA fragment were fused into pgsA-HA1 through a bridge PCR reaction using primers pgsA-F and HAI-R, which contained Spe I and Hnd III sites, respectively.

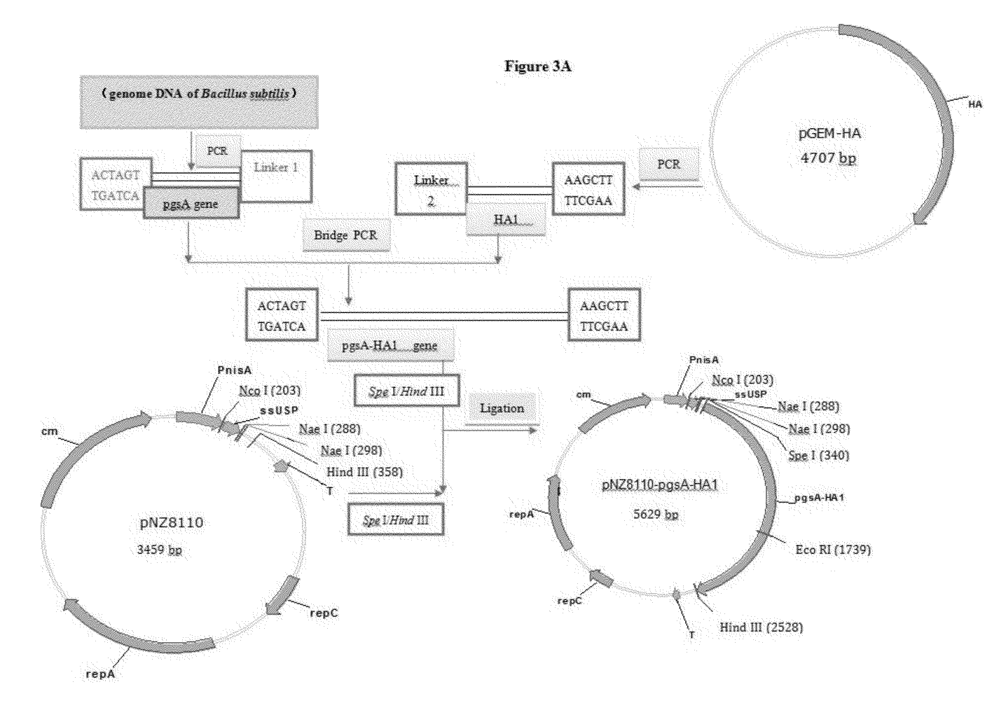

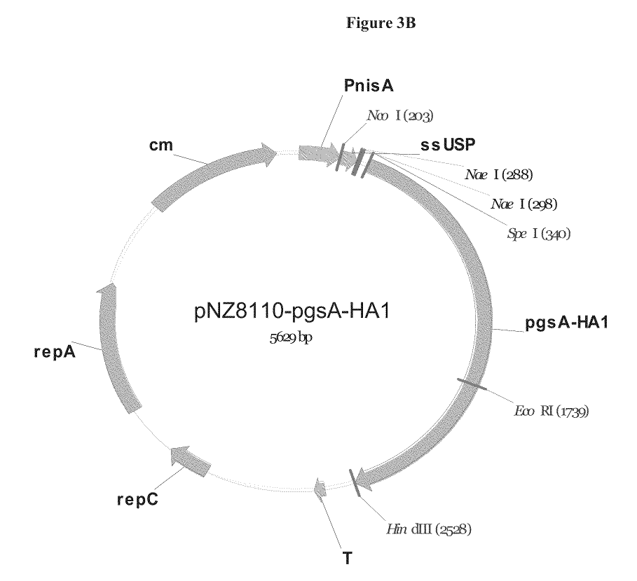

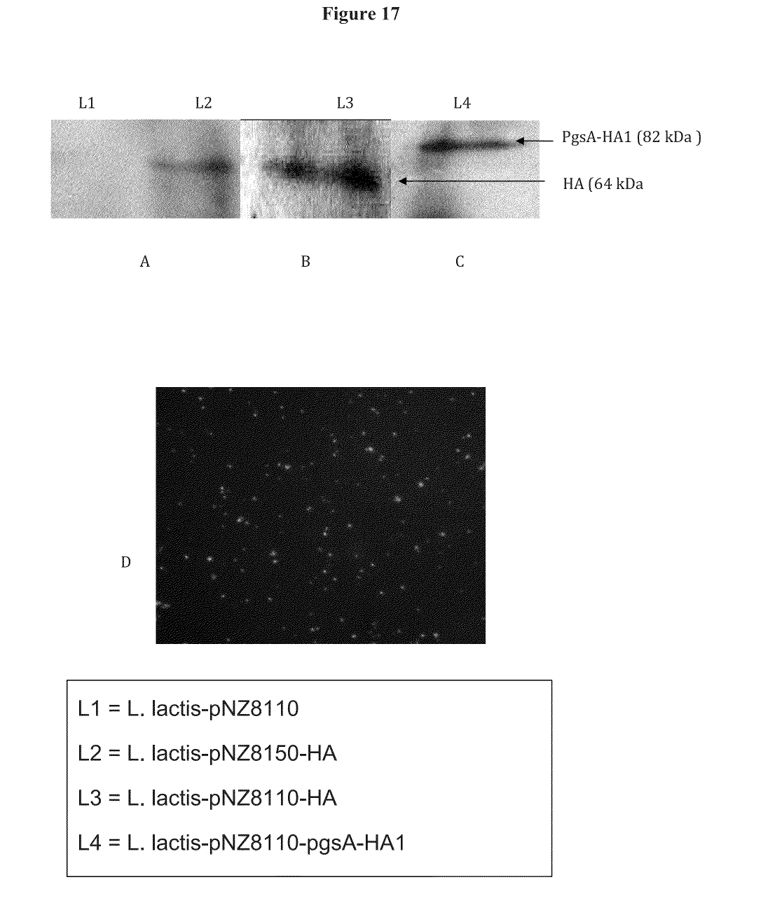

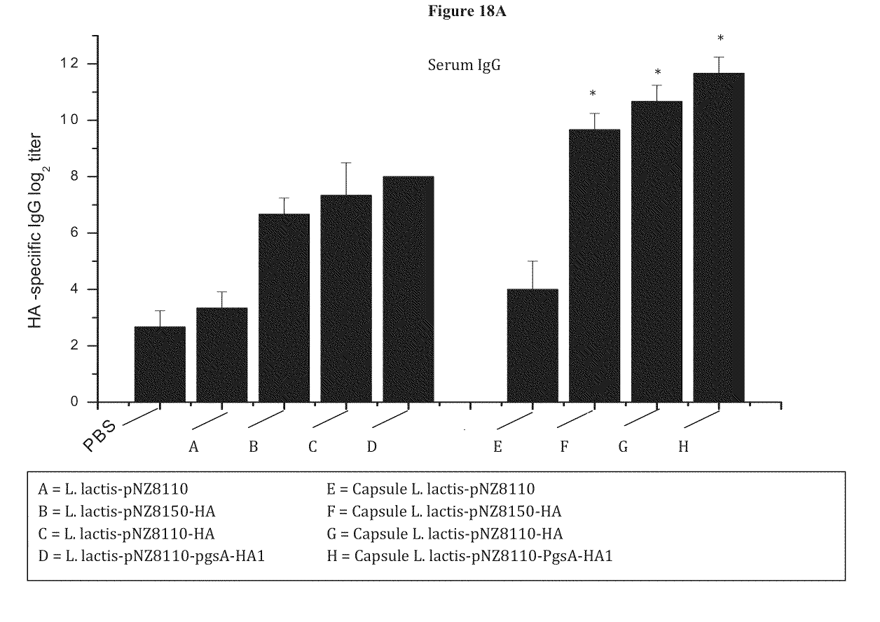

For generation of surface display plasmids, the resultant pgsA-HA1 DNA fragment was digested with Spe I-Hnd III and inserted into similarly digested pNZ8110 under transcriptional control of the nisA promoter to generate pNZ8110-pgs-HA1 (FIG. 17). The creation of this construct is shown in FIG. 3A. The restriction map of the recombinant plasmid construct is shown in FIG. 3B.

As a negative control used in various experiments throughout this specification, certain L. lactis strains were transformed with the pgsA anchor protein gene alone to generate L. lactis-pgsA.

These constructed plasmids are shown below in Table 3.

TABLE 3
Recombinant plasmids and bacterial nomenclature

| | | | Antigen |
|---|---|---|---|
| Plasmid | Bacteria | Antigen | location |
| pNZ8110 | L. lactis-pNZ8110 | None | |
| (pNZ8148 with an | | | |
| usp45 signal | | | |
| sequence) | | | |
| pNZ8150-HA | L. lactis-pNZ8150-HA | HA | cytoplasm |
| (HA gene cloned in | | | |
| E. coli-L. lactis | | | |
| shuttle vector | | | |
| pNZ8148) | | | |
| pNZ8110-HA | L. lactis-pNZ8110-HA | HA | secreted |
| (HA gene cloned in | | | |
| pNZ8110) | | | |
| pNZ9700(HA) | L. lactis-pNZ9700(HA) | HA | secreted |
| (plasmid from | | | |
| selected clone of | | | |
| pNZ8110-HA) | | | |
| pNZ8110-PgsA-HA1 | L. lactis-pNZ8110- | HA1 | cell wall |
| (PgsA-HA1 fusion | PgsA-HA1 | | anchored |
| gene clone in | | | |
| pNZ8110) | | | |
| PgsA (no plasmid) | L. lactis-pgsA | None | |
| (DNA sequence for | | | |
| PgsA anchor | | | |
| protein) | | | |

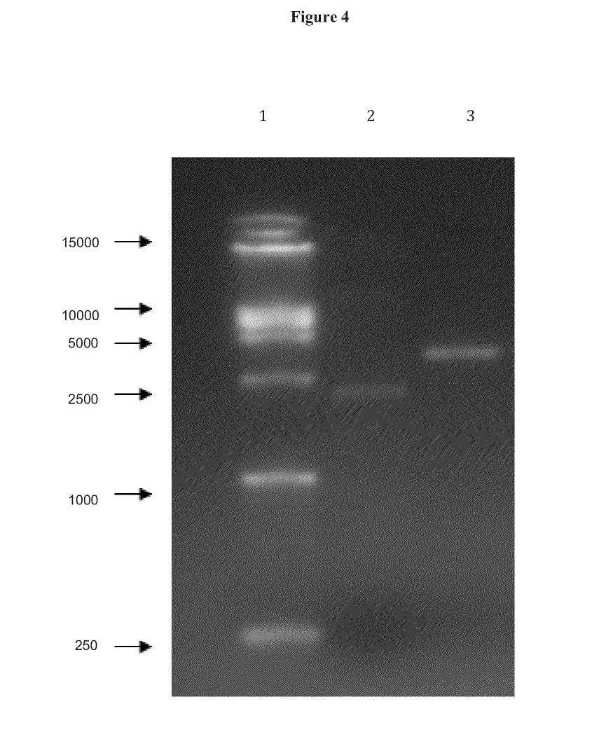

An electrophoresis analysis of the plasmid pNZ8110 is shown in FIG. 4, wherein double restriction endonuclease cutting at Nae1/HindIII sites is shown.

### Example 2

### Materials and Methods

The following experiments in Example 2 concern pNZ8110-HA, L. lactis-pNZ8110-HA (and clone Llactis-NZ9700(HA)) alone.

### Transformation

L. lactis NZ9700 was purchased from NIZO (Netherlands) and cultured in M17 broth medium (Difco, Sparks, Md., USA) containing 0.5% (WN) glucose (GM17) at 30° C. overnight without agitation. The L. lactis NZ9700 transfected with the pNZ8110-HA plasmid by electroporation using a Gene Pulser (Bio-Rad) at 25 uF, 1000V with 0.1-cm electrode cuvette (Bio-Rad).

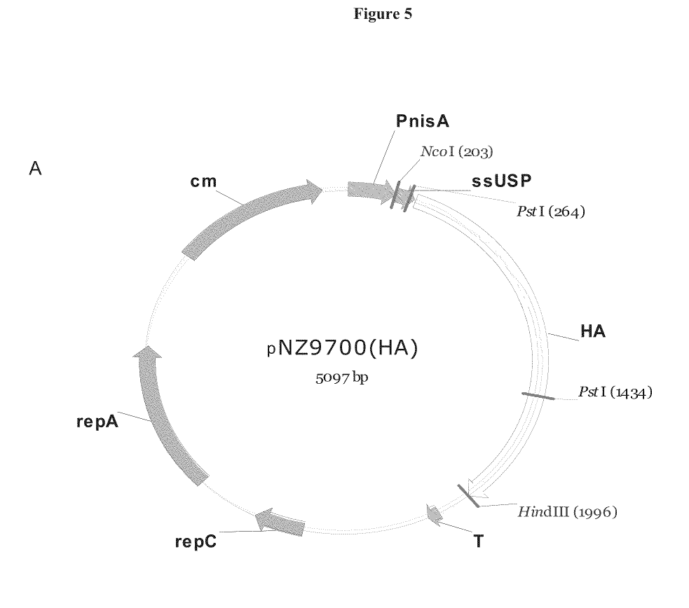

The highest HA expressing clone was selected and named Llactis-NZ9700(HA) (plasmid shown in FIG. 5). As a negative control, L. lactis NZ9700 was transformed with empty vector pNZ8110 (NIZO, Netherlands) to generate L. lactis-pNZ8110.

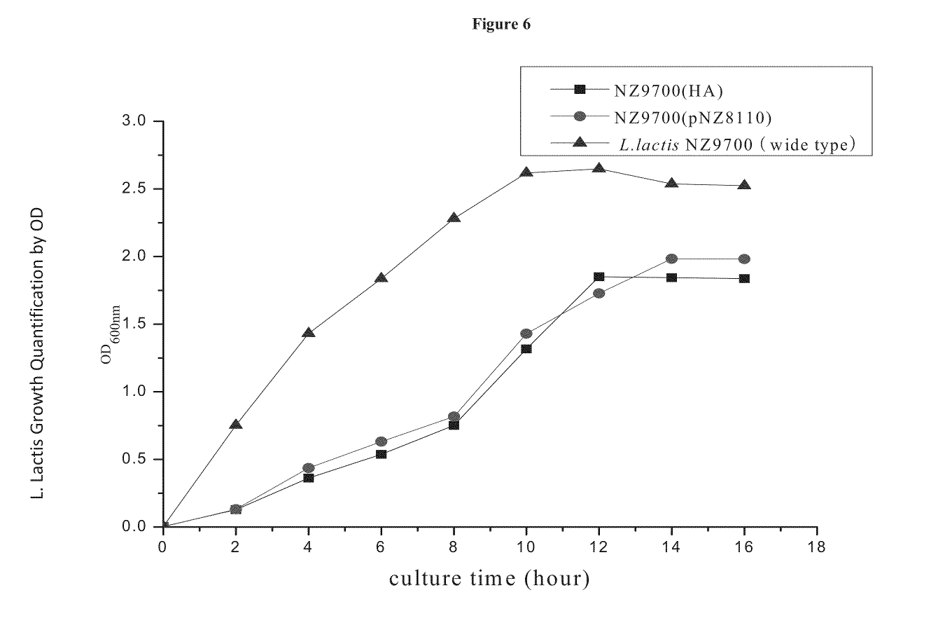

An analysis of the rate of growth of the cultures of L. lactis-pNZ9700(HA), L. lactis-p-NZ8110 and L. lactis NZ9700 (wild type) (FIG. 6).

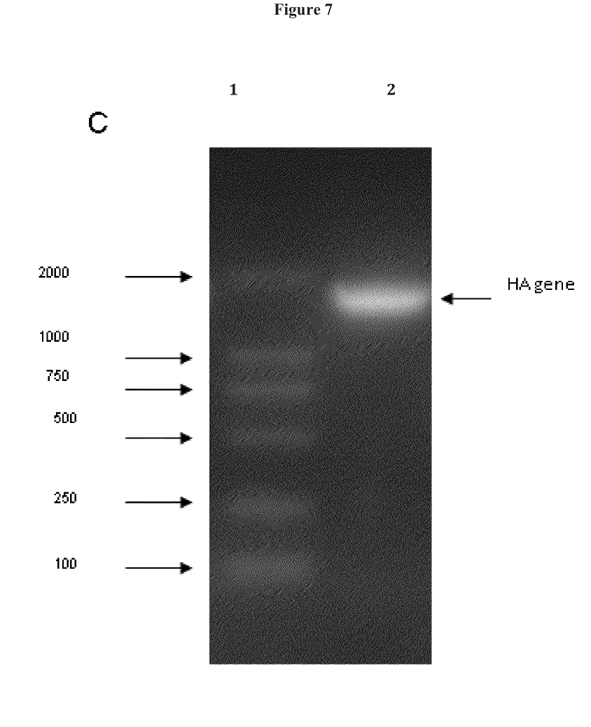

Plasmid DNA was isolated from L. lactis NZ9700 for PCR detection and sequencing of the target gene. and PCR detection of the HA sequence in the NZ9700(HA) clone were shown in FIG. 7 respectively. The full length HA gene (1704 bp) was sequenced after PCR amplification and proved to be correct.

HA Antigen Expression by L. lactis-pNZ9700(HA) In Vitro

To confirm the expression of the HA gene insert, L. lactis were cultured in GM17 medium with 10 μg/ml chloramphenicol at 30° C. overnight without agitation. The cultures were centrifuged at 5000×g for 8 minutes at 4° C. Pellets were washed twice with wash buffer in phosphate-buffered saline [PBS], and bacteria were suspended in equal volumes of 2× sodium dodecyl sulfate (SDS) buffer (125 mM Tris[tris(hydroxymethyl) aminomethane]-HCl, pH 6.8, 4% SDS, 20% glycerol, 0.01% bromophenol blue, and 10% 3-mercaptoethanol). After boiling for 10 minutes, the cell lysates were electrophoresed on 4% concentration gel and 10% separated polyacrylamide gel. Another gel was transferred to a nitrocellulose membrane. Protein was detected using mouse anti-HA antibody followed by affinity-purified horseradish peroxidase (HRP)—conjugated goat anti-mouse IgG. The membrane was radiographed on X-film using the ECL Western Blotting Detection System. The L. lactis-pNZ8110 was used as a control.

### Alginate Microcapsule Preparation

Alginate microcapsules were prepared to encapsulate L. lactis-pNZ9700(HA) to facilitate oral administration and to protect the live vectors from the acidic environment in the stomach. Concentrated sodium alginate solution was added to the L. Lactis culture medium and mixed well. Soybean oil containing 0.2% of Tween-80 was then mixed with the solution to form an emulsion with constant stirring (540 rpm) for 10 min Calcium chloride solution (1% w/v) was gradually infused into the emulsion until the emulsion collapsed. The suspension was then subjected to 3000 g centrifugation to collect the microcapsules. The pellet was resuspended, washed with deionized water, and then lyophilized.

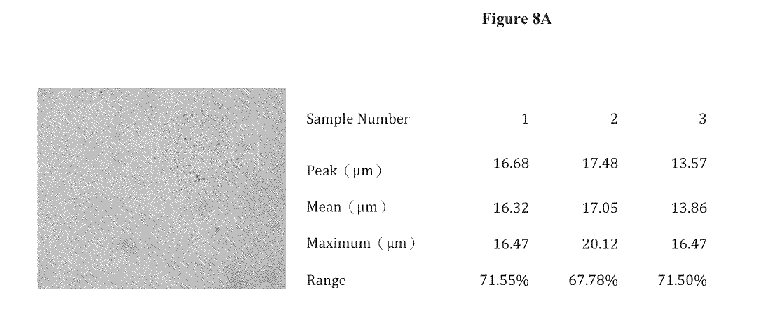

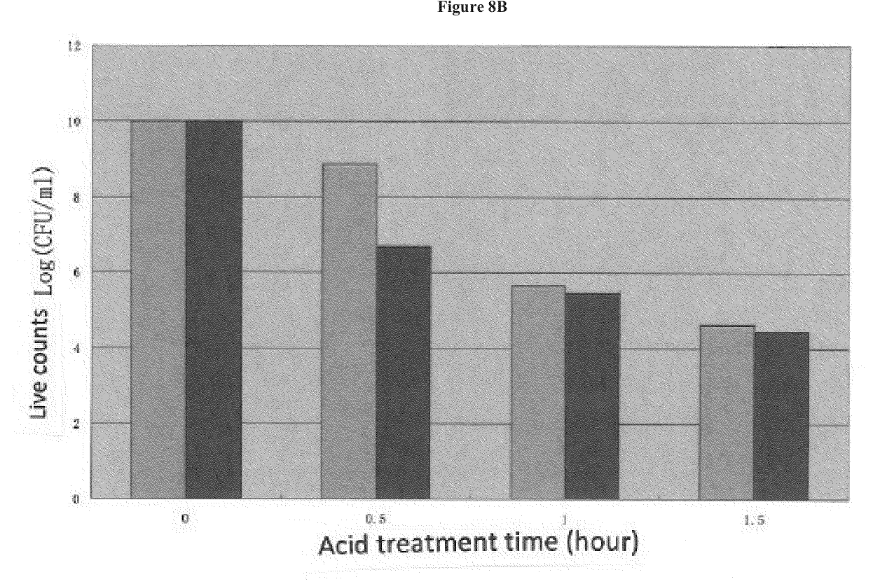

FIG. 8A showed a microscopic picture of an alginate capsule containing L. Lactis. The size distribution of these capsules were around 15 um. The protective effect on L. Lactis by these microcapsules against an acidic environment was demonstrated in FIG. 8B.

### Enteric Coated Capsule Preparation

L. Lactis culture medium was mixed with dextran and lyophilized. The lyophilized powder was filled into enteric coated capsules.

### Oral Immunizations in Mice

Eight-week-old BALB/c female mice were purchased from SLC, Inc. (Shanghai, China) and housed in the Animal Center of the school of pharmacy of the Shanghai Jiao Tong University. The mice were kept under standard pathogen-free (SPF) conditions and provided with free access to food and water during the experiments.

Immune Responses Induced by Oral Vaccination with L. lactis-pNZ9700 (Ha) in Mice Analyzed Through Antibody Titers Analysis

A set of mouse experiments were performed, wherein 10¹⁰ colony-formation units (CFUs) of L. lactis were administered orally to groups of 4 BALB/c mice (SPF grade) 5 or 9 times over a 8-week period in two vaccination regimens. The clone L. lactis-pNZ9700(HA) was used to induce a response, while the bacteria L. lactis-pNZ8110 was used as a control.

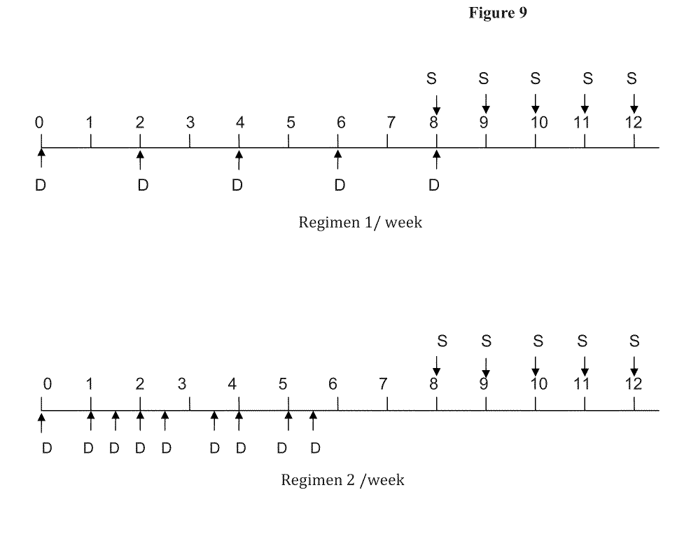

The vaccination regimens are shown in FIG. 9, where D(↑) denotes dosing and S(↓), sampling. In Regimen 1, the CFUs were dosed to the relevant mouse group upon the initiation of the experiment, at week 2, week 4, and week 6. In Regimen 2, the CFUs were dosed to the relevant mouse group upon the initiation of the experiment, twice in week 1 (at the start and in the middle of the week), twice in week 2 (at the start and in the middle of the week), once in week 3 (in the middle of the week), once in week 4 (at the start of the week), and twice in week 5 (at the start and in the middle of the week).

Samples of sera (collected by retro-orbital puncture) were collected starting 8 weeks from the start of the experiment, and were collected once time at the start of the week for five weeks thereafter (at the start of weeks 8, 9, 10, 11, and 12). Sera was stored at −20° C. until use.

Fecal pellets were collected one time at the tenth week after the initiation of immunization. Fecal pellets (100 mg) were suspended in 0.5 mL sterile PBS.

After centrifugation at 12 000×g for 5 minutes, the supernatants of either the sera or the fecal pellets were collected and tested for IgG or IgA, respectively, by ELISA.

The Enzyme-linked immunosorbent assay (ELISA) was performed as described elsewhere (Röhm, C., et al., 1995, Virology 209, 664-670). Briefly, 96-well microtiter plates were coated with mouse anti-HA antibody overnight at 4° C. The wells were blocked with PBS-1% bovine serum albumin (BSA) and incubated for 2 hours at room temperature. Serially diluted serum or fecal suspensions (100 ul) were added for 1 hour at 37° C. Bound Ab was detected using HRP-conjugated goat anti-mouse IgG or HRP-conjugated goat anti-mouse IgA. The ELISA end-point titers were expressed as the highest dilution that yielded an optical density (OD) greater than the two times mean OD₄₅₀ₙₘ plus S.D. of similarly diluted negative control samples.

### Hemagglutinin Inhibition (HAI) Assay to Show Neutralizing Antibody Titers in Mice

The HA neutralization ability of IgG antibodies generated after oral vaccination was examined using the classic hemagglutinin inhibition (HAI) assay. Sera was treated with receptor destroying enzyme (RDE) to inactivate non-specific inhibitors of agglutination, prior to being tested. Briefly, three parts RDE were added to one part sera and incubated overnight at 37° C. Samples were then heat-inactivated at 56° C. for 30 min. Following inactivation, PBS was added to the sample for a final serum dilution of 1:10. The diluted samples were then stored at 4° C. prior to testing (up to 6 days) or stored at −20° C.

Chicken Red Blood Cells (CRBC) were adjusted with PBS to obtain a 1% (v/v) suspension and kept at 4° C. until use within one week of preparation. Mice serum samples were diluted serially in two-fold dilutions using v-bottom microtiter plates. An equal volume of H5 subtype standard antigen, adjusted to approximately 8 HAU/50 μl, was added to each well. The plates were covered and incubated at room temperature for 30 min followed by the addition of 1% CRBC. The plates were mixed by agitation, covered, and the CRBC were allowed to settle for 30 min at room temperature. The HAI titer was determined by the reciprocal dilution of the last row that contained non-agglutinated CRBC. Positive and negative ferret serum controls were included for each plate. HI titers of 2³ or higher were counted as positive.

Mouse sera taken at 10 weeks after the first dosing in regimen 1 were tested.

### Results

HA Protein Expression by L. lactis-pNZ9700 (HA) In Vitro

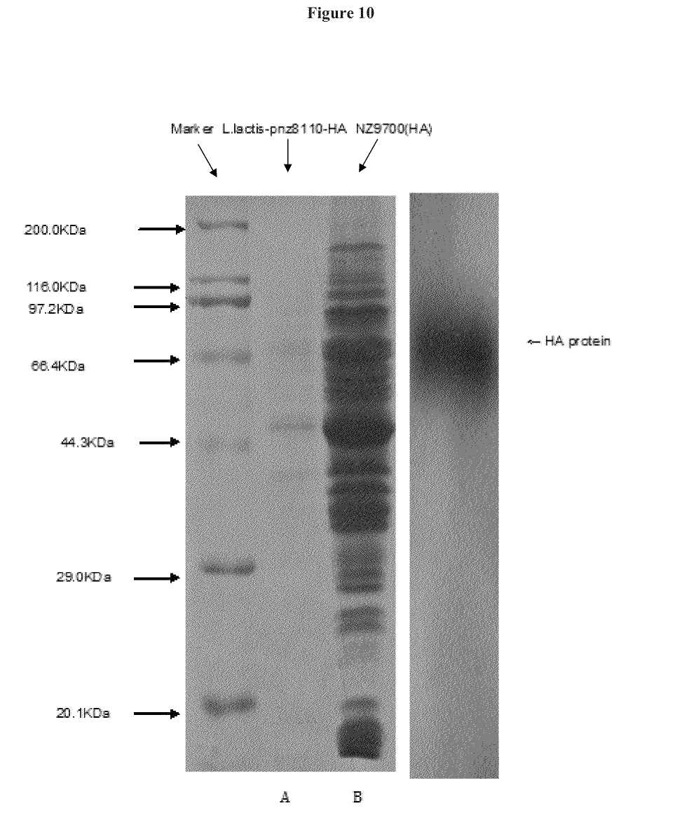

To evaluate whether the encoded protein was actively produced by the L. lactis-pNZ9700(HA) bacteria, SDS-PAGE and Western blotting analysis were done as described under Materials and Methods. The expression of the HA protein (66.2 kDa) was detected by SDS-PAGE in L. lactis-pNZ9700(HA) lysates but not L. lactis-pNZ8110 lysates (FIG. 10A). The culture supernatant was also analyzed by Western blot and the HA band was clearly visible (FIG. 10B), indicating HA proteins were expressed and secreted outside of the L. lactis due to the presence of the secreting signal Usp45.

Immune Responses Induced by Oral Vaccination with L. lactis-pNZ9700(Ha) in Mice

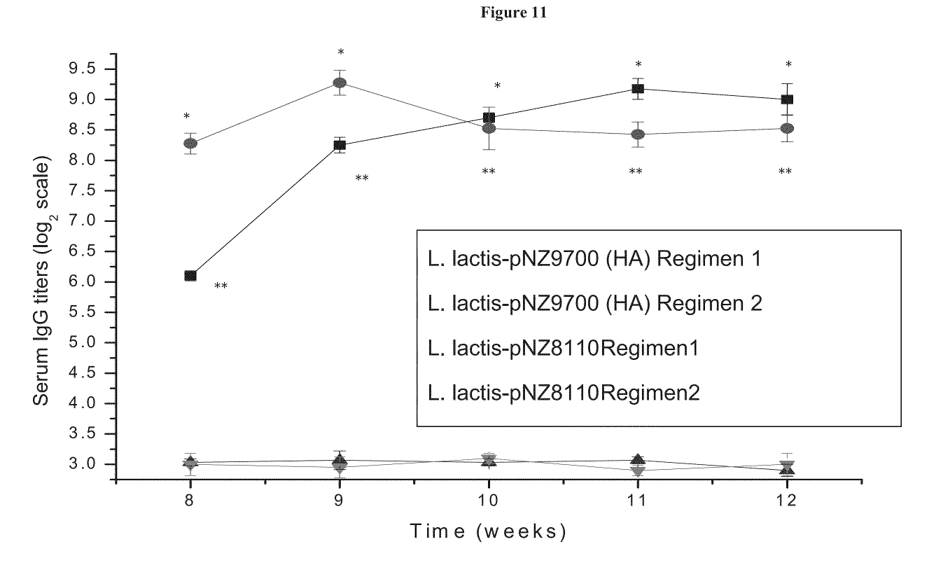

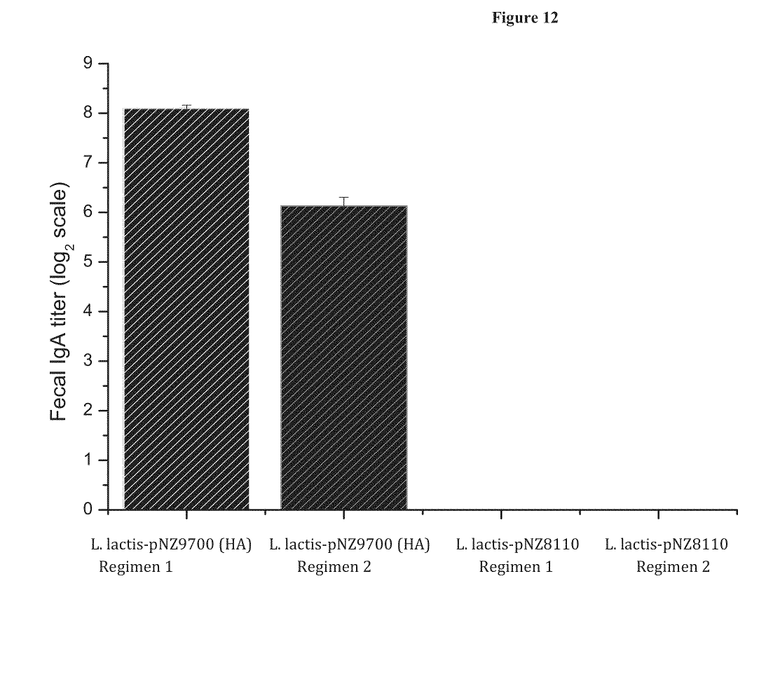

The effect of vaccination on the production of HA-specific serum IgG and fecal IgA antibodies was examined 5 weeks after the final immunization. Both IgG and IgA responses were detected in both groups immunized with L. lactis-pNZ8110-HA, while those treated with L. lactis-pNZ8110 had no HA specific antibody at all (see FIGS. 11 and 12). As shown in FIG. 11, serum IgG titers were significant after 4 biweekly oral doses and remained high for at least several months afterwards. An increase in the number and frequency of the oral doses as in regimen 2 did result in faster IgG generation, but the final titers were similar. The maxima dilution of serum IgG was 2^{9.4} (regimen 1).

### Hemagglutinin Inhibition (HAI) Assay

The data from the hemagglutinin inhibition (HAI) assay is listed in Table 4. The geometric mean HI titer was 2⁷ in L. lactis-pNZ9700(HA) treated group, compared to the background level (<2³) in the L. lactis-pNZ8110 group.

TABLE 4
Hemagglutinin inhibition titers of vaccinated
mouse sera against H5 subtype standard antigen

| | HI titer before |
|---|---|
| Group | challenge^{a}(GMT) |
| L. lactis-pNZ9700 (HA) | 2⁷ |
| L. lactis-pNZ8110 | <2³ |

GMT: geometric mean titer.
^{a}HI titer of 2³ was recorded as positive.

### Example 3

### Materials and Methods

The following experiments in Example 3 concern challenge experiments involving a comparison of clone L. lactis-pNZ9700(HA) vis-à-vis control bacteria L. lactis-pNZ8110, as well as L. lactis-pNZ8110-HA vis-à-vis a wild type control.

### Protection Against Lethal H5N1 Virus Challenge

Eight-week-old female BALB/c mice were used in all experiments. Influenza A virus (A/chicken/Henan/12/2004(H5N1)) was used for the virus challenge. Fifty percent mouse infectious dose (MID50) and 50% lethal dose (LD50) titers were determined. To test whether L. lactis-pNZ8110-HA inoculated mice could stand against a H5N1 virus challenge, lethal challenge experiments were performed at the tenth week after five biweekly oral dosings. To evaluate the degree of protection from lethal challenges, vaccinated mice were infected intranasally (i.n.) with 10 LD50 of Influenza A virus (A/chicken/Henan/12/2004(H5N1)) virus (lethal challenge dose). Six mice from each group were examined daily for survival for 21 days.

### Protection Against Lethal H5N1 Virus Challenge

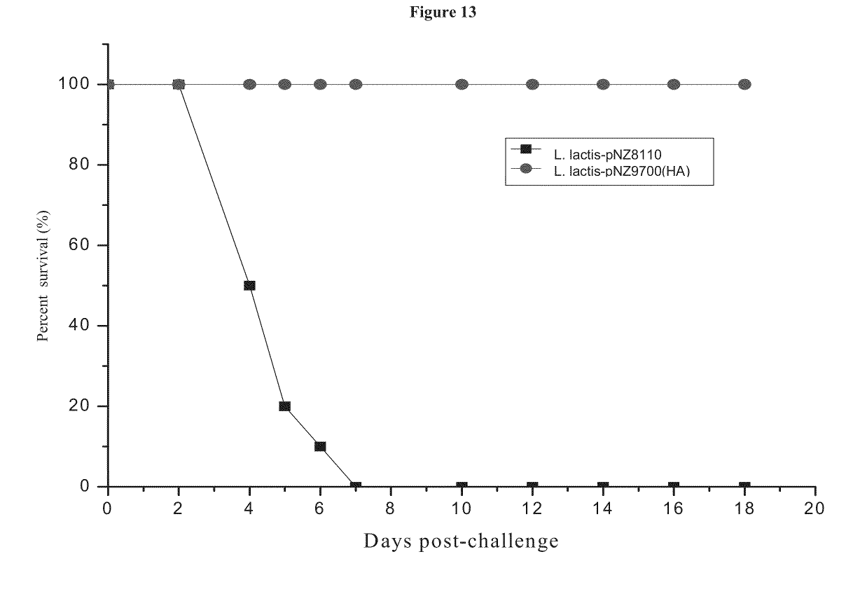

The results indicated that mice immunized with L. lactis-pNZ9700 (HA) were protected completely (6/6) after being challenged with a lethal dose (10 LD50) of H5N1 virus, and the percent survival rate was 100%. The control group [treated with L. lactis-pNZ8110] however was killed completely within seven days (see FIG. 13).

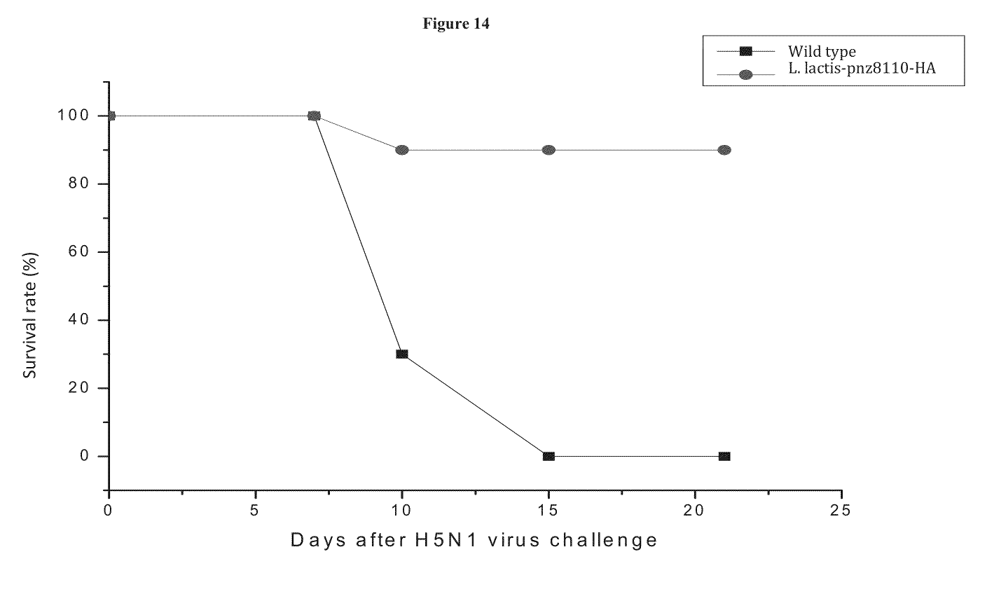

The results also indicated improved survival among mice immunized with L. lactis-pNZ8110-HA after being challenged with a lethal dose (10 LD50) of H5N1 virus, and the percent survival rate was 90%. The control group [treated with WTNZ9700] was killed completely withi 15 days (see FIG. 14).

### Example 4

### Materials and Methods

The following experiments in Example 4 concern experiments involving a comparison of effectiveness of immunization with L. lactis-pNZ8110, L. lactis-pNZ8150-HA, L. lactis-pNZ8110-HA, and L. lactis-pNZ8110-PgsA-HA.

### Oral Immunizations in Mice

Eight-week-old BALB/c female mice were purchased from SLC, Inc. (Shanghai, China) and housed in the Animal Center of the school of pharmacy of the Shanghai Jiao Tong University. The mice were kept under standard pathogen-free (SPF) conditions and provided with free access to food and water during the experiments.

After overnight fasting, groups of ten mice received 200 μl 10¹⁰ CFU (colony-formation units) of L. lactis using an oral zonde needle. Mice were immunized at days 0˜3, 7˜10, and 21˜24. Sera was taken at day 34. Fecal pellets were also collected on day 34.

### Antibody Titers Analysis

Samples of sera (collected by retro-orbital puncture) and feces were collected 2 weeks after final immunization. Sera were stored at −20° C. until use. Fecal pellets (100 mg) were suspended in 0.5 mL sterile PBS. After centrifugation at 12 000×g for 5 minutes, the supernatants were collected and tested for IgG or IgA by ELISA.

The Enzyme-linked immunosorbent assay (ELISA) was performed as described elsewhere (Röhm, C., et al., 1995, Virology 209, 664-670). Briefly, 96-well microtiter plates were coated with mouse anti-HA antibody overnight at 4° C. The wells were blocked with PBS-1% bovine serum albumin (BSA) and incubated for 2 hours at room temperature. Serially diluted serum or fecal suspensions (100 μl) were added for 1 hour at 37° C. Bound Ab was detected using HRP-conjugated goat anti-mouse IgG or HRP-conjugated goat anti-mouse IgA. The ELISA end-point titers were expressed as the highest dilution that yielded an optical density (OD) greater than the two times mean OD₄₅₀ₙₘ plus S.D. of similarly diluted negative control samples.

### Results

HA-specific serum IgG was determined by ELISA and are shown in FIG. 15. * and ** represent statistically significant differences relative to the PBS control (*p<0.05, ** p<0.01). Data are expressed as the mean value (log 2) ±S.D. of duplicate experiments.

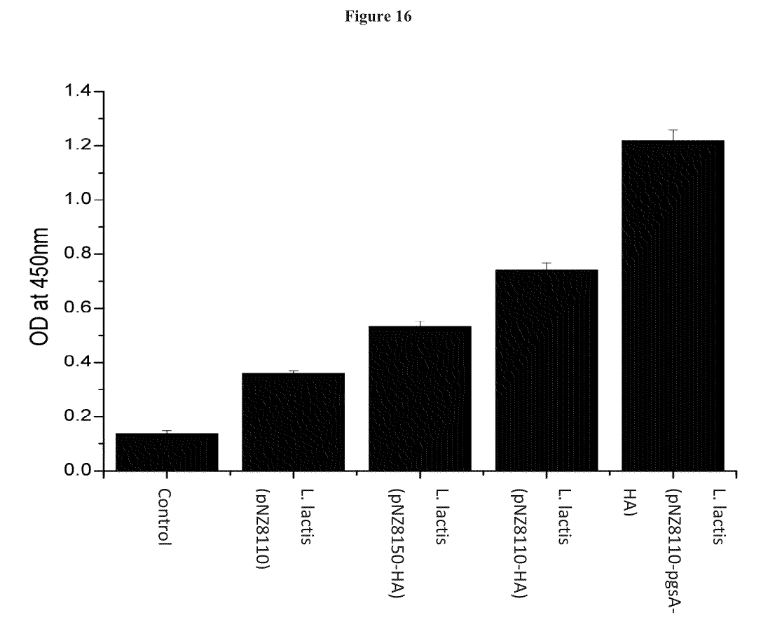

HA-specific mucosal IgA was detected by ELISA and are shown in FIG. 16. * and ** represent statistically significant differences relative to the PBS control (*p<0.05, ** p<0.01). Data are given as mean±SD of duplicate experiments.

### Example 5

The following experiments in Example 5 concern immunization experiments with L. lactis-pNZ8110, L. lactis-pNZ8150-HA, L. lactis-pNZ8110-HA, and L. lactis-pNZ8110-PgsA-HA.

### Materials, Methods, and Results

Recombinant L. lactis Vectors

The vectors of Table 3 were made as described. Relevant plasmids were transformed into the L. lactis NZ9000 strain by electroporation to create L. lactis-pNZ8110, L. lactis-pNZ8150-HA, L. lactis-pNZ8110-HA, and L. lactis-pNZ8110-pgsA-HA. The most highly expressed clones were selected and cultured at 30° C. in media based on M17 medium supplemented with 0.5% (wt/vol) glucose. Chloramphenicol was used at a concentration of 10 μg/ml.

### Western Blot Analysis and Immunofluorescence Microscopy

The antigen expressions were induced in all the recombinant L. lactis strains by adding nisinA to the final concentration of 10 ng/ml. Growth was continued for 3 hours after the addition of nisinA. For the L. lactis-pNZ8110, L. lactis-pNZ8150-HA, and L. lactis-pNZ8110-PgsA-HA1 cultures, L. lactis cells were harvested, washed three times with 500 ul sterile phosphate-buffered saline (PBS), and resuspended. Aliquots of the samples were mixed with 6× loading buffer and boiled 10 minutes. Extracts were run on an SDS-PAGE (10% acrylamide) gel and transferred to a polyvinylidene difluoride membrane (PVDF, Millipore, USA). Antibody reactions and detection (enhanced chemiluminescence) were performed according to the manufacturer's recommendations (Amersham Bioscience, Sweden). For the L. lactis-pNZ8110-HA culture, the supernatant was collected for the western blot analysis. For the immunofluoresence staining, the L. lactis-pNZ8110-PgsA-HA1 cells were harvested after induction, and incubated with polyclonal mouse anti-HA antibody at 4° C. for 30 minutes followed by fluorescein isothiocyanate (FITC)-conjugated anti-mouse IgG. The resulting cell pellets were examined using an Olympus fluorescence microscope.

Enteric Capsule Preparation and L. lactis Release in Simulated Gastrointestinal (GI) Environment

About 10⁹ CFU of the recombinant L. lactis vectors were packaged in an enteric capsule (mini-capsule), each measuring about 4×1 mm in size. The samples were named capsule L. lactis-pNZ8110, capsule L. lactis-pNZ8150-HA, capsule L. lactis-pNZ8150-HA, and capsule L. lactis-pNZ8110-pgsA-HA1. The capsule samples as well as solution samples were treated with simulated gastric fluid at pH 1.0 for 2 hours and then released in simulated intestinal buffer at pH 6.8 for 45 minutes. The resultant live L. lactis cell counts (CFU) were listed in Table 5. The capsule groups showed strong acid resistance and survived at pH 1.0, while in the solution groups the live vector counts showed massive loss of viability (>20000-fold) after the treatments.

TABLE 5
The relative live L. lactis count after
simulated gastric fluid treatment

| | live bacteria |
|---|---|
| L. lactis vector | number (CFU) |
| L. lactis-pNZ8110 | 3.5 × 10⁴ |
| L. lactis-pNZ8150-HA | 3.6 × 10⁴ |
| L. lactis-pNZ8110-HA | 3.8 × 10⁴ |
| L. lactis-pNZ8110-pgsA-HA1 | 3.6 × 10⁴ |
| capsule-L. lactis-pNZ8110 | 10⁹ |
| capsule-L. lactis-pNZ8150-HA | 10⁹ |
| capsule-L. lactis-pNZ8110-HA | 10⁹ |
| capsule-L. lactis-pNZ8110-pgsA-HA 1 | 10⁹ |

### Animals and Animal Immunizations

Six-week-old female BALB/c mice were purchased from China SLC, Shanghai, China. The mice were housed in the specific pathogen-free (SPF) Animal Center of Shanghai Jiao Tong University. Before each dose, they were fasted for 6 hours and then administer 10 ul of the recombinant L. lactis solution or 1 capsule using a 21-gauge feeding tube Immunizations were repeated at 2, 4, 6 weeks after the initial dosing.

L. lactis-pNZ8110-PgsA-HA1 was representatively chosen for the chicken experiments, seven-day-old female SPF chickens were inoculated by subcutaneous injection and oral immunization at the doses of 10⁸CFU/chicken Immunizations were given at days 1 and 2, 7, 14, 21, 28, 29.

ELISA Assay for HA-Specific IgG and IgA Responses after Oral Administration of Solutions or Capsules

Sera from mice or chickens were collected 10 days after the last dosing. HA-specific antibody responses were detected by avidin-biotin system (ABS)-enzyme-linked immunosorbent assay (ELISA) (Katz, J. M. et al., 1997, J. Infect. Dis. 175, 352-363). 10 μg/ml Influenza A virus (A/chicken/Henan/12/2004(H5N1)) recombinant HA protein was used to coat 96-well microplates. At the same time, Fecal pellets (50 mg) for each group of six mice were collected and suspended in 250 μl sterile PBS, centrifuged down at 15 000× rpm for 10 minutes, and the supernatants tested for IgA by indirect ELISA. The mean antibody titer was expressed as the highest dilution that yielded an optical density greater than twice the mean plus one standard deviation of that of similarly diluted negative control samples.

### H5N1 Virus Challenge

Eight-week-old female BALB/c mice were used in all experiments. Influenza A virus (A/chicken/Henan/12/2004(H5N1)) was used for the virus challenge. Fifty percent mouse infectious dose (MID50) and 50% lethal dose (LD50) titers were determined as previously described (Grangette, C. et al., 2002, Vaccine 20, 3304-3309). To evaluate the degree of protection from lethal challenges, vaccinated mice were infected intranasally (i.n.) with 10 LD50 of Influenza A virus (A/chicken/Henan/12/2004(H5N1)) virus (lethal challenge dose). Six mice from each group were examined daily for survival for 21 days.

All values are expressed as means standard error (SE). Statistical analysis of the experimental and control data were determined by using Student's t-test or analysis of variance (ANOVA). Significance was defined as a P value less than 0.05. For survival, the probability was calculated by using Fisher's exact test, comparing the rate of survival in mice immunized with the recombinant L. lactis to that of the control groups.

### IFN-γ ELISpot Assay

The IFN-γ ELISpot assay was performed one week after the final immunization using an ELISpot kit for mouse IFN-γ as recommended by the manufacturer (R&D Systems, USA). Briefly, mouse IFN-γ microplate was added 200 μl/well of sterile culture media and incubated for 20 minutes at room temperature. After aspirating the culture media from the wells, to the plates were added 100 μl of 1×10⁶ splenocytes per well. 10 μg/ml of HA-specific peptide (ISVGTSTLNQRLVP) was used as stimuli for 48 hours in a humidified 37° C. CO₂ incubator. Control wells were not stimulated with HA-specific peptide. After incubation, each well was aspirated and washed, the plates were treated sequentially with biotinylated anti-mouse IFN-γ antibody, alkaline phosphatase conjugated strepavidin and the substrate solution to reveal the spots. The developed microplate could be analyzed by counting spots using a dissection microscope.

### Neutralization Assay

Determination of endpoint neutralizing antibody titers was performed by microneutralization assay, as previously described (Rowe, T., et al., 1999, J. Clin. Microbiol. 37, 937-943). Briefly, serial 2-fold dilutions of sera treated with receptor-destroying enzyme (RDE) from Vibro cholerae were mixed and incubated with 35 μl 100 50% tissue culture infective doses (TCID₅₀) of H5N1 virus, then added to Madin-Darby canine kidney (MDCK) cells and incubated for 1 h. The H5N1 virus-infected MDCK cells were further cultured for 72 h at 37° C. in the presence of 5% CO2, and the neutralizing titer was determined by hemagglutination test. For the HA test, 50 μl of 0.5% cock red blood cells was added to 50 μl of cell culture supernatant and incubated at room temperature for 30 min. The TCID₅₀ was determined on the basis of the Reed-Muench method (Röhm, C., 1995, Virology 209, 664-670). The neutralization titer (IC₅₀) was defined as the reciprocal of the antiserum dilution at which H5N1 virus entry was 50% inhibited.

### H5N1 Virus Challenge

For the challenge experiment, mice were anesthetized and intranasally challenged with 20 μl 10×50% lethal dose (LD₅₀) H5N1 virus two weeks after the last immunization. After infection, the mice were weighed and monitored for signs of illness for 14 days. The challenge experiments were strictly performed under biosafety level-3-plus enhancement conditions.

### Statistical Analysis

Statistical analysis of the experimental and control data were performed by one-way factorial analysis of variance. P-values less than 0.05 were considered as a statistical significance.

### Results

### Western Blot Analysis and Immunofluorescence Microscopy

Western blot analysis showed the L. lactis-pNZ8110 as a control vector which doesn't express any HA (FIG. 17A), and L. lactis-pNZ8150 expressed the HA protein (64 kDa) which was mostly found in the cell lysate (FIG. 17A). L. lactis-pNZ8110-HA has an usp45 signal sequence cloned before the HA gene, so expressed HAs were mostly found in the culture supernatant (FIG. 17B). L. lactis pNZ8110-pgsA-HA 1 contained a plasmid encoding the HA1 protein (38 kDa) fused with the PgsA surface display motif (44 kDa). The resultant protein expressed had 82 kDa MW (FIG. 13C). In addition, the cell wall anchored distribution of the HA1 protein (in L. lactis pNZ8110-pgsA-HA1) was confirmed by immunofluorescence staining (FIG. 17D).

HA-specific IgG and IgA Responses after Oral Administration of Solutions or Capsules

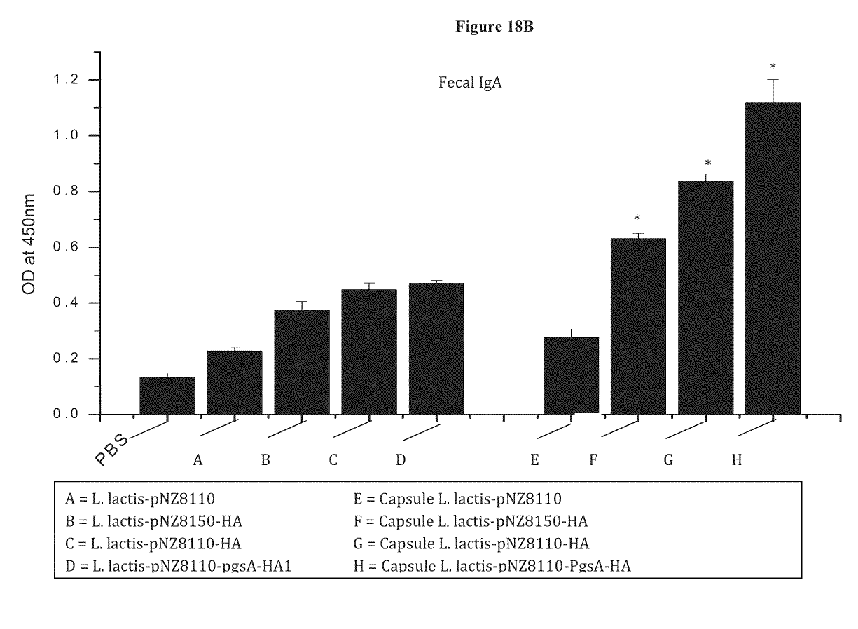

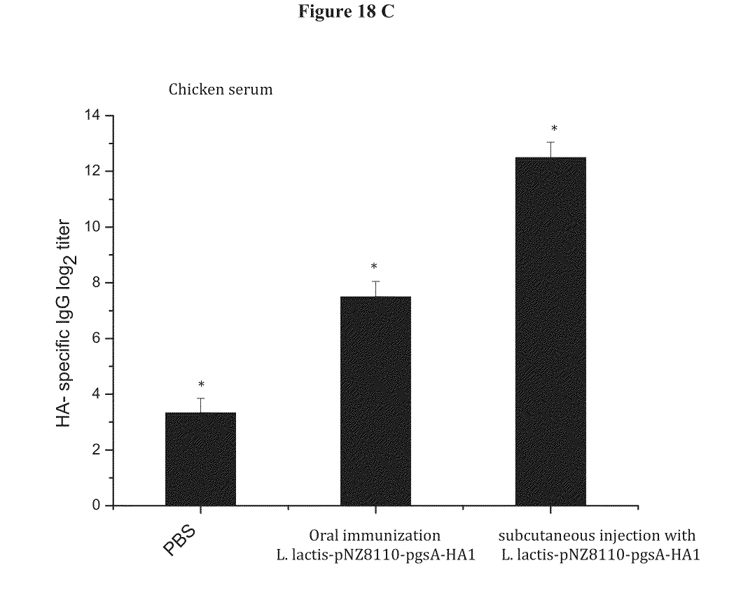

HA-specific serum IgG and fecal IgA levels were measured 10 days after the last immunization dosing. The mean log₂ titers of serum IgG of all the tested groups were shown in FIG. 18A. All the HA expressing vectors (solution and capsules) resulted in significant production of HA-specific serum IgG, while PBS and L. lactis-pNZ8110 samples did not. In general, encapsulated groups had higher titers than the solution groups. The group dosed with capsule L. lactis-pNZ8110-pgsA-HA1 reached the highest antibody titer. To examine the HA-specific mucosal immune response, the production of mucosal IgA antibody was examined using fecal pellets (FIG. 18B). Again, the enteric capsule groups all developed significant IgA antibody responses. The capsule-pNZ8110-pgsA-HA1 gave the best results. In experiments where seven-day-old chickens were inoculated either by subcutaneous injection or by oral administration at days 1 and 2, 7, 14, 21, 28, 29, sera were analyzed 10 days after the final immunization. Both delivery mechanisms yielded significant serum IgG titers, although chickens treated by subcutaneous injection were somewhat higher than the orally immunized groups (FIG. 18C).

### IFN-γ ELISpot Assay

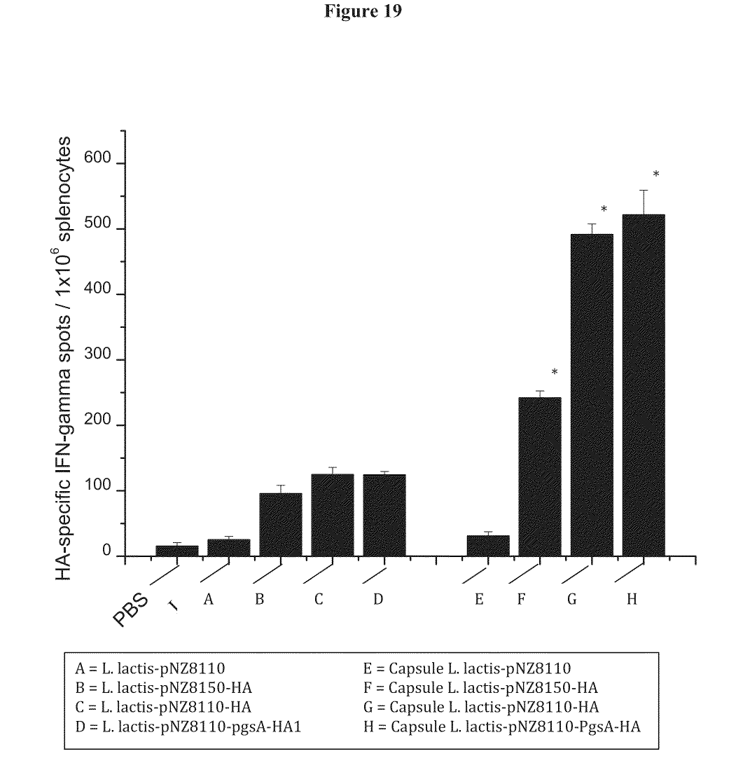

The IFN-γ ELISpot assay was also used to examine the HA-specific T cell response resulted from recombinant L. lactis after oral administration. Splenocytes (10⁶ cells) from each treatment group were collected and stimulated with 10 μg/ml of HA epitope peptide (ISVGTSTLNQRLVP). The resulting IFN-γ expressing T cell numbers were counted and plotted in FIG. 19. Again, there were HA-specific T cell responses generated by oral administration of enteric coated recombinant L. lactis. The encapsulated groups (capsule-L. lactis-pNZ8150-HA, capsule L. lactis-pNZ8110-HA and capsule L. lactis-pNZ8110-pgsA-HA1) were much more effective than the respective solution groups.

### H5N1 Virus Challenge Experiment

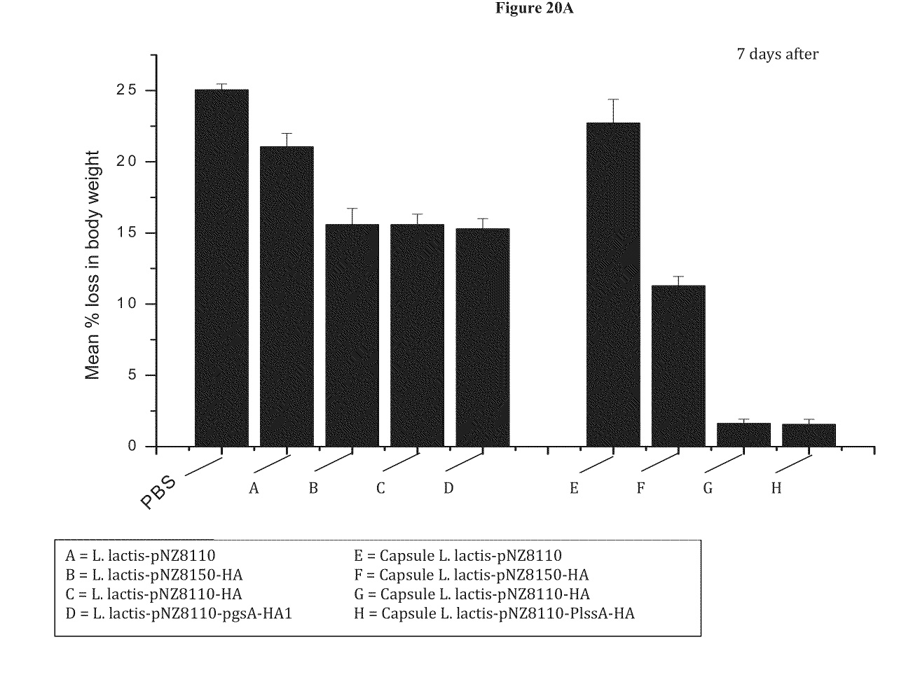

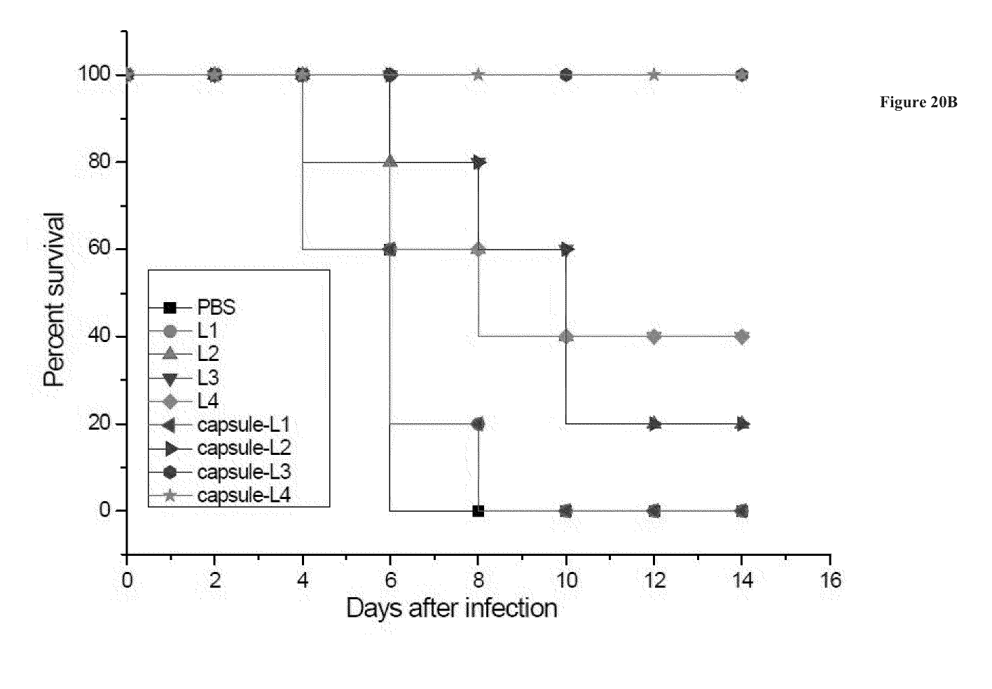

Two weeks after the final immunization, the mice were intranasally challenged with lethal doses of highly pathogenic H5N1 viruses and closely monitored for 14 days for weight loss and mortality. After viral challenge, all mice experienced certain levels of body weight loss (FIG. 20A), but mice immunized with capsule L. lactis-pNZ8110-HA and capsule L. lactis-pNZ8110-pgsA-HA1 gradually recovered after 8 days and were at 100% survival. In contrast, the naïve mice (PBS treated) and mice immunized with the empty plasmid vector (capsule L. lactis-pNZ8110) all died within 10 days of challenge (FIG. 20B).

### Neutralization Assay Results

The neutralizing antibody titers in each treatment groups were measured using microneutralization (MN). Neutralization titers values of the enteric capsule groups (capsule-L. lactis-pNZ8150-HA, capsule L. lactis-pNZ8110-HA and capsule L. lactis-pNZ8110-pgsA-HA1) were all higher than 80, suggesting good neutralization activities of the antibodies generated (Table 6).

TABLE 6
Neutralization assay of H5N1 virus with immune serum

| | Neutralization titer |
|---|---|
| Mice orally immunized with | of serum (IC₅₀) |
| PBS | <10 |
| L. lactis-pNZ8110 | <10 |
| L. lactis-pNZ8150-HA | 47 |
| L. lactis-pNZ8110-HA | 68 |
| L. lactis-pNZ8110-pgsA-HA1 | 70 |
| capsule-L. lactis-pNZ8110 | <10 |
| capsule-L. lactis-pNZ8150-HA | 80 |
| capsule-L. lactis-pNZ8110-HA | 148 |
| capsule-L. lactis-pNZ8110-pgsA-HA1 | 167 |

### Example 6

### Further Refinement

To adjust or lower the dose of bacteria used in the present invention, standard in vitro or in vivo titration experiments can be done, using various biological responses or animal survival described above as experimental readouts.

The same kind of titration experiments can also be done to determine the effects of the size of the coated capsule on immune response induction. Capsules of various size can be tested in the in vitro or in vivo experiments described above to investigate the effects on the induction of cellular immune responses, mucosal immune responses, and/or protective immune responses.

Similarly, the same kind of titration experiments can also be done to determine the effects of using different coating material or mucoadhesive polymers. Capsules coated with different coating materials, or bacteria formulated with different mucoadhesive polymers can be tested in the in vitro or in vivo experiments described above to investigate the effects on the induction of cellular immune responses, mucosal immune responses, and/or protective immune responses.

### Example 7

### The Construction of Recombinant L. lactis Strain Expressing and Displaying the Avian Influenza HA1 Antigens on its Surface

In the present invention, a recombinant L. lactis vector encoding the hemagglutinin 1 (HA1) gene of influenza virus H5N1 was constructed; the recombinant vector was designed to include PgsA, a C-terminal anchor protein from Bacillus subtilis, designed to ensure the presence of the HA1 protein on the bacterial cell wall. L. lactis strains were transformed with said vector in order to produce bacterial vectors possessing the HA1 protein on the bacterial cell surface. When said bacterial vectors were administered to mice in conjunction with adjuvant Cholera Toxin B (CTB), the inoculated mice demonstrated statistically significant rates of survival upon challenge with lethal doses of H5N1, as compared with control groups inoculated with PBS, of bacterial vectors without CTB or the HA1 protein (L. lactis-pNZ8110-pgsA-HA1 and L. lactis-pgsA).

### Plasmid Constructs and Transformation

As noted previously, a 1136 bp fragment containing the pgsA gene was amplified from the chromosomal DNA of B. subtilis by polymerase chain reaction (PCR) using primers pgsA-F (5′-ggactagt atgaaaaaagaactgagctttcatg-3′ (SEQ ID NO: 55)) and pgsA-R (5′-tcctcctggggatccagattttagtttgtcactatg-3′ (SEQ ID NO: 56)), wherein the Spe I site is underlined. The 1053 bp HA1 gene fragment encoding the HA1 protein containing the sequence encoding the globular domain of HA was amplified from [A/chicken/Henan/12/2004(H5N1) [GenBank accession number: AY950232] using the following primer pairs with Knd III sites underlined (HA1-F: 5′-ggatccccaggaggagatcagatttgcattggt-3′ (SEQ ID NO: 57), HA1-R: 5′-cccaascttttatctcttttttc-3′ (SEQ ID NO: 58)). Then the pgsA DNA fragment and HA1 DNA fragment were fused into pgsA-HA1 through a bridge PCR reaction using primers pgsA-F and HAI-R, which contained Spe I and Hnd III sites, respectively.

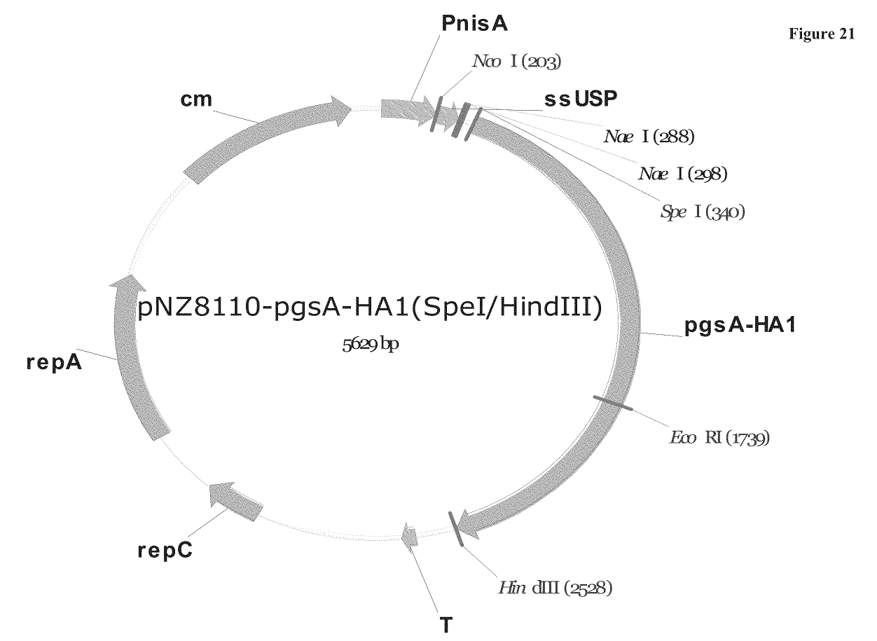

For generation of surface display plasmids, the resultant pgsA-HA1 DNA fragment was digested with Spe I-Hnd III and inserted into similarly digested pNZ8110 under transcriptional control of the nisA promoter to generate pNZ8110-pgs-HA1 (FIG. 21). The resultant plasmid was transformed into L. Lactis NZ9000 by electroporation and named L. lactis-pNZ8110-pgsA-HA1. As a negative control, L. lactis NZ9000 was transformed with the pgsA gene to generate L. lactis-pgsA.

### Nisin-Controlled Antigen Expression

Recombinant L. lactis cells were cultured in M17 broth medium with chloramphenicol 10 μg/ml containing 0.5% (WN) glucose (GM17) at 30° C. overnight without agitation. Overnight cultures of recombinant L. lactis were diluted 1:25 in fresh GM17 medium containing 10 μg/ml chloramphenicol. When an OD₆₀₀ₙₘ of 0.3˜0.4 was reached, nisinA was added at a final concentration of 10 ng/ml. Growth was continued for 3 h.

### Western Blot Analysis

To confirm the expression of the HA1 gene expression, L. lactis-pNZ8110-pgsA-HA1 and L. lactis-pgsA cells were harvested after induction, and centrifuged at 5,000×g for 8 minutes at 4° C. Cell pellets were washed twice with 20 mM Tris-HCl (pH 8.0) and the cell lysates and supernatants analyzed by Western blot. Each fraction was mixed with equal volumes of 2× sodium dodecyl sulfate (SDS) buffer (125 mMTris [tris(hydroxymethyl aminomethane]-HCl, pH 6.8, 4% SDS, 20% glycerol, 0.01% bromophenol blue, and 10% 3-mercaptoethanol). After boiling for 10 minutes, the cell lysates were electrophoresed on a 10% acrylamide gel and transferred to a nitrocellulose membrane. Protein was detected using polyclonal mouse anti-HA antibody followed by affinity-purified horseradish peroxidase (HRP)-conjugated goat anti-mouse IgG

### Flow Cytometric Analysis and Immunofluorescence Microscopy

L. lactis-pNZ8110-pgsA-HA1 and L. lactis-pgsA cells were analyzed to ascertain whether HA1 protein was expressed on the bacterial surface. L. lactis-pNZ8110-pgsA-HA1 and L. lactis-pgsA cells were centrifuged at 5,000×g for 10 minutes at 4° C., and washed three times with sterile PBS. The bacteria were then incubated with polyclonal mouse anti-HA antibody at 4° C. overnight followed by biotinylated goat anti-mouse IgG and phycoerythrin (PE)-conjugated streptavidin. The stained cells were analyzed by flow cytometry (BD FACSCalibur™ USA).

For immunofluorescence staining, the L. lactis-pNZ8110-pgsA-HA1 and L. lactis-pgsA cells were harvested after induction, and incubated with polyclonal mouse anti-HA antibody at 4° C. for 30 minutes followed by fluorescein isothiocyanate (FITC)-conjugated anti-mouse IgG L. lactis-pgsA was used as a negative control Immunofluorescence analysis was examined using a fluorescence microscope (OLYMPUS 1X71, Japan).

### Oral Immunizations in Mice

Six-week-old BALB/c female mice were purchased from SLC, Inc. (Shanghai, China) and housed in the Animal Center of the school of pharmacy of the Shanghai Jiao Tong University. The mice were kept under standard pathogen-free (SPF) conditions and provided with free access to food and water during the experiments.

After overnight fasting, groups of ten mice received 150 μl 10¹⁰ CFU (colony-formation units) of L. lactis-pNZ8110-pgsA-HA1 or L. lactis-pgsA plus 1 mg cholera toxin B (CTB) unit that was purchased from Sigma as a recombinant protein using an oral zonde needle. Other groups were administered with the same dose of L. lactis-pNZ8110-pgsA-HA1 and L. lactis-pgsA Immunization was performed daily via intragastric lavage on days 0 to 3, 8 to 11 and 24 to 28.

### ELISA Assay

Samples of sera (collected by retro-orbital puncture) and feces were collected at day 38 after final immunization. Sera were stored at −20° C. until use. Fecal pellets (50 mg) were suspended in 0.3 ml sterile PBS. After centrifugation at 12,000×g for 5 minutes, the supernatants were collected and tested for IgA by Enzyme-linked immunosorbent assay (ELISA). The IgG detection was performed by avidin-biotin system (ABS)-ELISA, and the IgG antibody was assayed by indirect-ELISA.

96-well plates were coated with recombinant influenza A virus (A/chicken/Henan/12/2004(H5N1)) HA protein overnight at 4° C. The wells were blocked with PBS-1% bovine serum albumin (BSA) and incubated for 2 hours at room temperature. 2-fold serially diluted serum or fecal suspension (100 ul) was added for 1 hour at 37° C. Bound Ab was detected using biotin-conjugated goat anti-mouse IgG, alkaline phosphatase-conjugated streptavidin (R&D Systems, USA) or HRP-conjugated goat anti-mouse IgA (Sigma). The mean Ab titer was expressed as the reciprocal of the serial serum dilution that reached the cut-of value plus 2 SD.

### ELISpot Assay

IFN-γ-producing cells were quantified one week after final immunization using an ELISpot kit (R&D Systems, USA) according to the manufacturer's instructions. To a mouse IFN-γ microplate was added 100 μl/well of 1×10⁶ splenocytes and 10 pg/ml of HA-specific peptide (ISVGTSTLNQRLVP) which was used as the stimulus for 48 hours in a humidified 37° C. CO₂ incubator. Control wells contained unstimulated cells. After incubation, each well was washed, the plates were incubated sequentially with biotinylated anti-house IFN-γ antibody, alkaline phosphatase conjugated streptavidin and the substrate solution to reveal the spots. The number of spots were counted by a dissection microscope. All assays were performed in triplicate.

### H5N1 Virus Challenge Experiments

Influenza A virus (A/chicken/Henan/12/2004/(H5N1)) was used for the virus challenge experiments. Two weeks after final immunization, mice (n=5 mice per group) were challenged intranasally with 5 LD50 of H5N1 virus in a volume of 20 μl. Mice were monitored for disease signs and death for 14 days after infection. All experiments were performed under high-containment biosafety level 3 enhanced (BSL3) conditions. Statistical analyses were performed using student's T test for data; p-values of <0.05 were considered a statistically significant difference.

### Results

Construction of Recombinant L. lactis-pgsA-HA1 and HA1 Protein Expression In Vitro

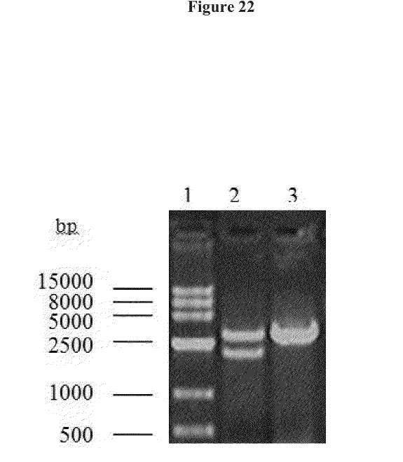

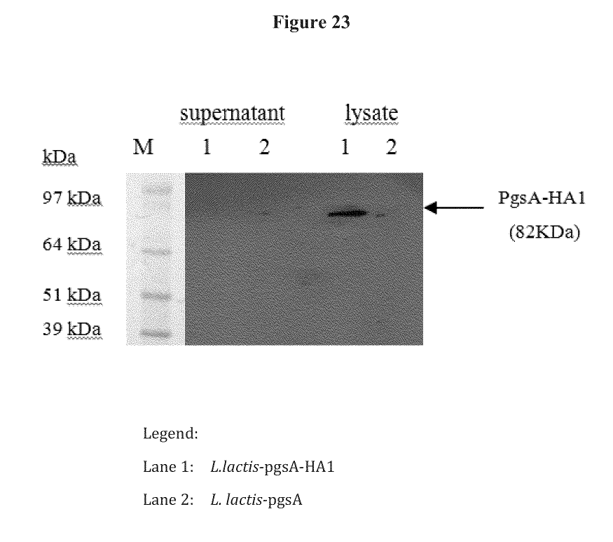

The pgsA and HA1 DNA fragments were fused in a bridge PCR system to generate pgsA-HA1 gene. After digestion, the pgsA-HA1 gene was transcriptionally fused with pNZ8110 to yield pNZ8110-pgsA-HA1 (FIG. 21). The resulting pNZ8110-pgsA-HA1 was transformed into the L. lactis NZ9000 strain. Purifed plasmids from recombinant L. lactis-pNZ8110-pgsA-HA1 and L. lactis-pgsA were detected by agarose gel electrophoresis analysis (FIG. 22). As shown in Lane 2 of (FIG. 22), pgsA-HA1 fragment (2189 bp) was successfully digested. Further western blot analysis of cell lysates and supernatants showed that HA1 was located on the surface of L. lactis-pNZ8110-pgsA-HA1 or intracellularly (FIG. 23). The band of 82 KDa most likely represents the HA1 protein (38 KDa) and the PgsA surface display motif (44 KDa).

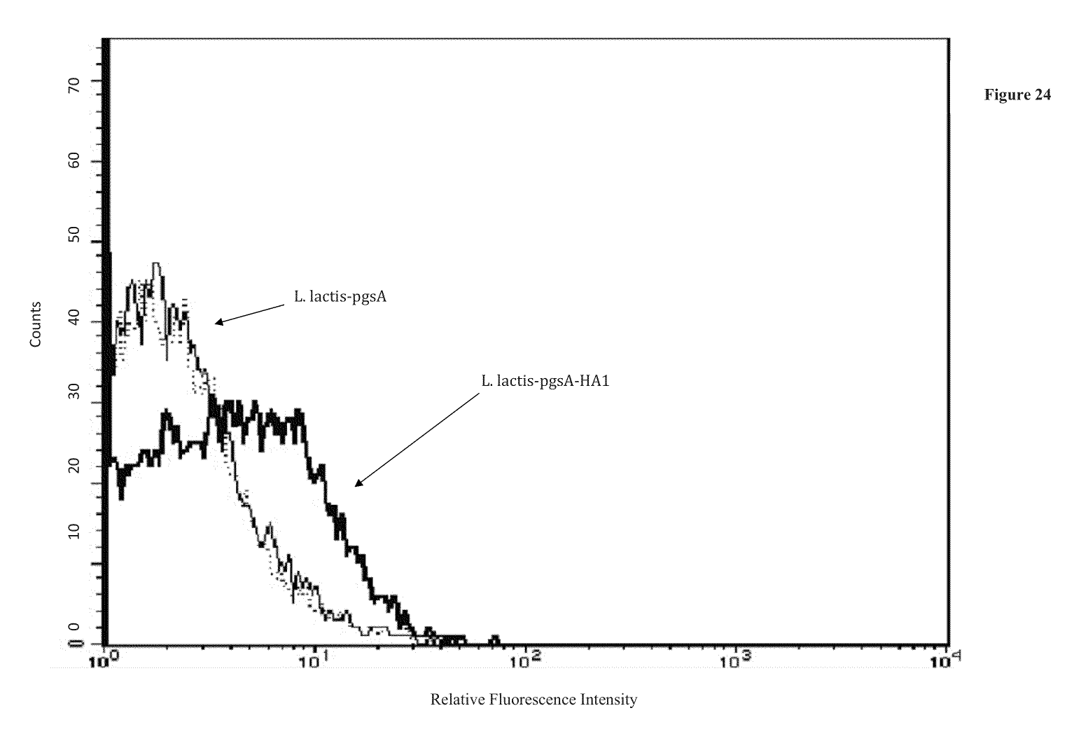

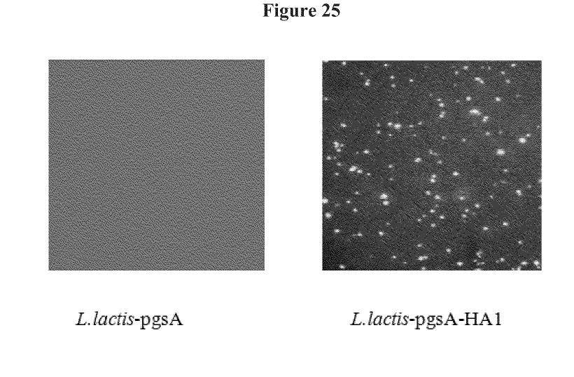

To determine if HA1 protein was expressed on the surface of L. lactis via cell wall anchor, FACS analysis revealed that a significant increase in fluorescence intensity was obtained in L. lactis-pNZ8110-pgsA-HA1 (FIG. 24). Immunofluorescence further showed that L. lactis cells harboring pNZ8110-pgsA-HA1 immunostained positive for HAL but control L. lactis cells harboring pgsA did not (FIG. 25). These data indicated the constructed recombinant L. lactis could make the HA1 effectively displayed on the surface of the cell wall.

Immune Responses Induced by Oral Vaccination with L. lactis-pNZ8110-PgsA HA1 and Adjuvant CTB in Mice

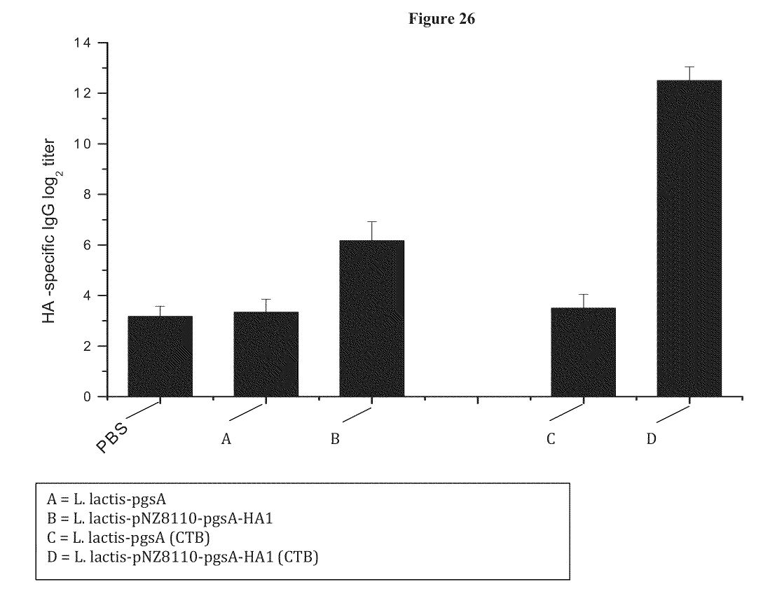

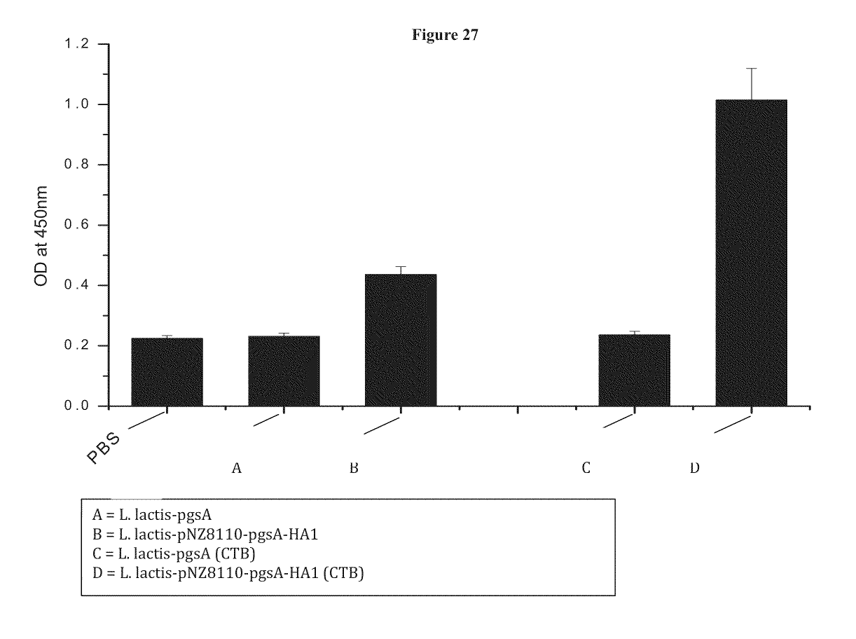

150 μL 10¹⁰ CFU of L. lactis-pNZ8110-pgsA-HA1 or L. lactis-pgsA with or without CTB were orally administered to BALB/c mice (SPF grade). The effects of vaccination on the production of HA-specific serum IgG and fecal IgA were examined at days 10 after the final immunization. As shown in FIG. 26, in mice orally administered with L. lactis-pNZ8110-pgsA-HA1 and CTB, serum IgG antibody titer had obtained a significant increase as compared with other groups. Fecal IgA was also examined and was shown to have been significantly produced after immunization with L. lactis-pNZ8110-pgsA-HA1 and CTB. See FIG. 27. These data demonstrated that mice administered with L. lactis-pNZ8110-pgsA-HA1 and CTB resulted in significant enhancements of HA specific IgG and mucosal IgA compared with controls of PBS, L. lactis-pgsA, L. lactis-pNZ8110-pgsA-HA1 and L. lactis-pgsA with adjuvant CTB.

L. lactis-pgsA-HA1 and CTB Immunization Stimulates HA-Specific Cellular Immune Response

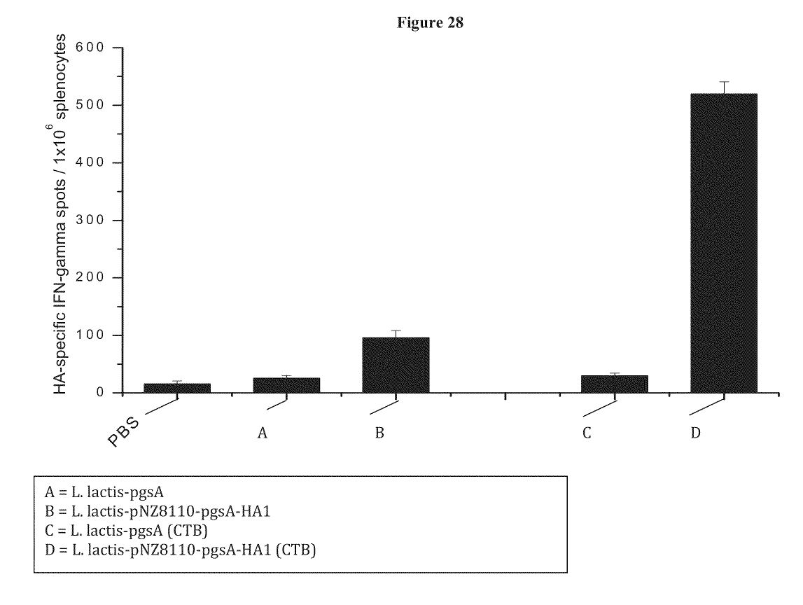

Lymphoproliferation and IFN-γ ELISpot assays were performed to determine whether L. lactis-pNZ8110-pgsA-HA1 and CTB could induce cellular immunity. Splenocytes were stimulated with 2 μg/ml recombinant HA protein and the proliferation levels were reflected by absorbance at 450 nm on a spectrophotometer. Compared to those from mice immunized with PBS, L. lactis-pgsA, L. lactis-pNZ8110-pgsA-HA1 or L. lactis-pgsA and adjuvant CTB, the number of HA-specific IFN-γ-secreting cells in L. lactis-pNZ8110-pgsA-HA1 CTB-treated mice, as shown by IFN-γ ELISpot assay was significantly enhanced (p<0.05, FIG. 28). These results suggested that oral administration with L. lactis-pNZ8110-pgsA-HA1 and CTB could effectively induce HA-specific cell-mediated responses (CMI).

### Protection Against Lethal H5N1 Virus Challenge

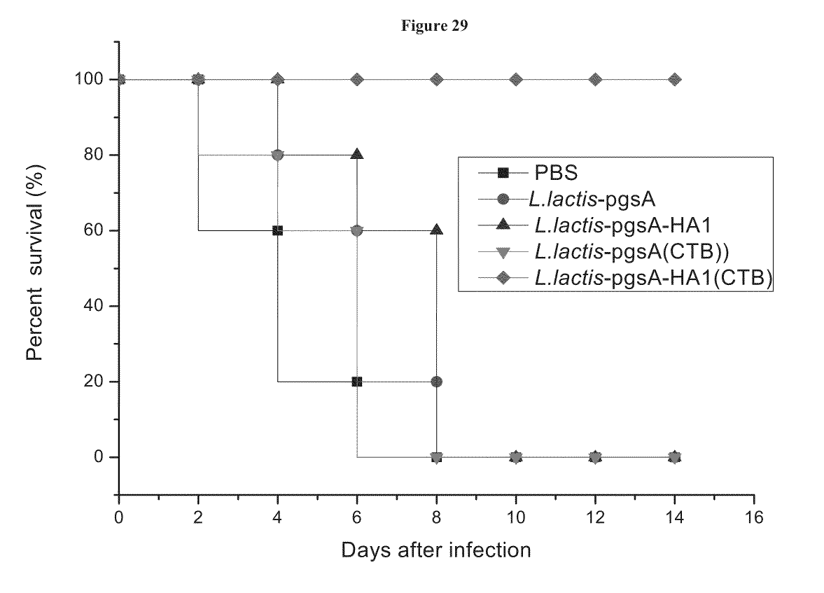

To test whether vaccinated mice could stand a H5N1 virus challenge, all mice (5 mice per group) were infected by intranasal drip with a lethal dose of 20 μl of 5 LK50. The survival rate was monitored for 14 days after H5N1 virus infection. The percentage survival among the various groups indicated that mice immunized with L. lactis-pNZ8110-pgsA-HA1 and CTB obtained 100% protection whereas, by contrast, other groups were killed within 10 days (FIG. 29). These data demonstrated L. lactis-pNZ8110-pgsA-HA1 and CTB is an effective vaccine for providing protection against H5N1 virus challenge.

### Example 8

### Agrobacterium-Mediated Transformation of A. thalianai Using Floral-Dip Method

### A. Plant Culture

1. Clip the primary inflorescences when most plants have formed primary bolts (about 3-4 weeks after planting the germinated seeds in soil).

2. Dip the plants when most secondary inflorescences are 1-10 cm tall (2-4 days after clipping)

3. Cover the plants dipped in Agrobacterium solution with the black plastic package to maintain humidity and leave them in a low-light or dark location overnight.

4. Remove dome and return plants to the growth chamber 12 to 24 hrs. after inoculation.

5. Dip the inflorescences again after 6-7 days if it is needed. The inflorescences can be dipped 3 times with 6 days between each application.

6. Allow plants to grow for a further 3-5 weeks until siliques are brown and dry.

7. Harvest seeds by gentle pulling of grouped inflorescences through fingers over a piece of clean paper.

8. Store seeds in microfuge tubes and kept at 4° C. under desiccation.

### B. Agrobacterium Culture

1. Inoculate Agrobacterium (LAB4404 or EHA105) in LB (30 g Kan+50 g Rif/mL) and shake overnight at 25° C.-28° C. with 250 rpm.

2. Transfer the overnight culture into new flasks with LB (30 g Kan+50 mg Rif/mL) and continue to shake 18-24 hours under the same conditions until the culture reaches stationary phase.

3. Harvest the cell by centrifugation for 20 min at room temperature at 6000 g and resuspend in floral dip solution to final OD₆₀₀ of approximately 0.80. The agrobacterium is ready for dipping now.

### C. Floral Dip Inoculation

1. Put the inoculum into a beaker and make plants inverted into this suspension such that all above-ground tissue is submerged.

2. Gently agitate the plants for 3 sec-5 sec. and move the plants out of the beaker.

3. Cover and treat the plants as above (3 in plant culture).

### D. Selection for Transformed Plants

1. Weigh the transformed seeds (about 1250 seeds=25 mg=50 L) and sterilize the seeds by treating them with 95% ethanol for 30-60 s and 20-50% of bleach (2.625% sodium hypochlorite, final volume) containing 0.03% of Tween 20 for 5 min Rinse the seeds 2-3 times with sterile water.

2. Suspend the sterilized seeds in approximately 10-20 mg seeds/mL of 0.1% (w/v) agarose and plate on 0.8% of agar selection plates (with 20-30 g Kan/mL) at a density of approximately 3000 seeds per 150×15 mm2 plate. The selection plates contain one-half-strength MS medium (Murashige-Skoog).

3. Seal the plates and cold-treat them for 2 days.

4. Grow for 12-15 days in a controlled environment at 24° C. under 23 h light 50-100μ Einsteins m-2 s-1. (Remove excess moisture during growth by briefly opening the plates and shaking moisture off the lid.)

5. Identify transformants as kanamycin-resistant seedlings that produce green leaves and well-established roots within the selective medium.

6. Grow some of the transformants to maturity by transplanting into heavily moistened potting soil, preferably after the development 3-5 adult leaves.

7. Genomic DNA of kanamycin resistant plants is amplified with specific primers to confirm the transgenic plants.

### E. Solution and Medium

1. Floral dip solution (1000 mL) is made as follows: 5% (w/v) of sucrose (add 100 mL of 50% sucrose), 10 mM MgC12 (optional) (add 10 mL of 1M MgCl2, 0.02-3% Silwet L-77 (add 200-300I (L), and add H2O to 1000 mL.

2. LB-kanamycin/rifampicin medium: LB broth with 30 g kanamycin and 50 l Rifampicin respectively.

Selection medium: % times of MS medium and 0.8% agar with 20-30 g of Kanamycin/mL.

### Example 9

### Demonstration of DNA Constructs in Animal Models

BALB/c mice (male) of 7 to 8 weeks of age were provided by the Laboratory Animal Unit of The University of Hong Kong. The mice were kept and fed by the animal technicians in the animal house of Department of Zoology, The University of Hong Kong.

### A. Design of Animal Inoculations

35 BALB/c mice were randomly divided into 7 groups; each group consisted of 5 mice. The approach was separately replicated twice (demonstration 1 and demonstration 2).

Details of the embodiment of the invention are shown in Table 7:

TABLE 7
Details of demonstration setup

| | Group # | Treatment |
|---|---|---|
| IBD vaccine | Group 1 | Injected with 100 g pcDNA2.1-VP5-5.2 & |
| | | VP2-3, 4 intramuscularly. |
| IBD vaccine | Group 2 | Fed with E. coli cells containing 100 g |
| | | pcDNA3.1-VP5-5.2 & VP2-3.4. |
| HC vaccine | Group 3 | Injected with 100 g pHCV2.5 intramuscularly. |
| HC vaccine | Group 4 | Fed with E. coli cells containing 100 g |
| | | pHCV2.5. |
| PRRS vaccine | Group 5 | Injected with 100 g pcDNA3.1-ORF5 |
| | | intramuscularly. |
| HC-PRRS | Group 6 | Injected with 100 g (pHCV2.5 and |
| combined | | pcDNA3.1-ORF5) intramuscularly. |
| vaccine | | |
| Control | Control | Without any treatment |
| | Group | |

In Groups 1, 3, 5 and 6, the mice were injected intramuscularly at the tibialis using 27-gauge needles. DNA vaccine was injected at a single site each time. For Groups 2 and 4, the mice were fed by using a feeding needle (18060-20, Fine Science Tools).

The vaccination scheme was the same for all the mice, and it was shown in the following tables.

TABLE 8
Vaccination scheme of mice in demonstration 1

| Day | Treatment |
|---|---|
| 0 | Bleeding & 1^{st} vaccination |
| 7 | Bleeding |
| 8 | 2^{nd} vaccination |
| 15 | Bleeding |
| 18 | 3^{rd} vaccination |
| 28 | Bleeding |

TABLE 9
Vaccination scheme of mice in demonstration 2

| Day | Treatment |
|---|---|
| 0 | Bleeding & 1^{st} vaccination |
| 8 | Bleeding |
| 10 | 2^{nd} vaccination |
| 17 | Bleeding |
| 19 | 3^{rd} vaccination |
| 28 | Bleeding |

### B. Blood Sampling and Serum Preparation

For the first three times, mice were bled by cutting small portion of their tails (−2 mm) and 200 μl of blood was collected. For the last time, mice were bled by cardiac puncture under ether anesthetic, and 700 μl of blood was bled using 27-gauge needle. Blood samples were allowed to clot by incubation at room temperature for 4 hours. The clotted blood was centrifuged at 5000 rpm for 10 minutes and the serum was collected.

### C. Growing CEF Cells

1 ml CEF cells (Chicken Embryo Fibroblasts) were thawed from liquid nitrogen. The cells were then resuspended in 10 ml Dulbecco's Modified Minimal Essential Medium (DMEM) with 10% heat-inactivated fetal calf serum (FCS, Gibco BRL) and 1% antibiotics (Penicillin-Streptomycin, Gibco BRC) and seeded in a T-75 flask (Falcon). The cells were incubated at 37° C. with 5% CO₂ overnight.

The 100% confluent CEF cell monolayer was washed with 1×PBS twice and detached by 0.05% trypsin EDTA (Gigco BRL) for 5 minutes. Trypsin was neutralized by 10% FCS in MEM and centrifuged at 1000 rpm for 5 minutes. The cell pellet was resuspended in DMEM with 10% FCS and 1% antibiotics (Penicillin-Streptomycine, Gibco BRC). The cells were seeded to two T-75 flasks and if needed, one T-175 flask in the split ratio of 1 to 3 for subculturing.

### D. Growing PIC 15 Cells/MARC-145 Cells

1 ml PK-15/MARC-145 cells was thawed from the liquid nitrogen. The cells were then resuspended in IOral Minimum Essential Medium (MEM) with 10% heat-inactivated fetal calf serum (FCS, Gibco BRL) and 1% antibiotics (Penicillin-Streptomycin, Gibco BRC) and seeded to at T-75 flask (Falcon). The cells were incubated at 37° C. with 5% CO 2 overnight.

The 100% confluent cell monolayer was washed with 1×PBS twice and detached by 0.05% trypsin EDTA (Gibco BRL) for 5 minutes. Trypsin was neutralized by 10% FCS in MEM and centrifuged at 1000 rpm for 5 minutes. The cell pellet was resuspended in MEM with 10% FCS and 1% antibiotics (Penicillin-Streptomycin, Gibco BRC). The cells were seeded to two T-75 fasks and if needed, one T-175 flask in the split ratio of 1 to 3 for subculturing,

E. Virus Purification from Tissue Culture

CEF, PK-15 and MARC-145 cell were used for the amplification of IBDV, HCV and PRRSV respectively. The cell lines were infected with the respective virus in DMEM (for CEF) or MEM (for PK-15 and MARC-145) supplemented with 10% heat-inactivated PBS for 5 days. Virus was released from the cells by freezing and thawing 3 times. Any cells attached on the culturing flasks were scraped off. Detached cells and cell debris were removed by centrifugation at 2000 rpm for 10 minutes. The supernatant containing the partially purified virus was then centrifuged at 30000 rpm for 2 hours with a Beckman 40-Ti rotor. The pellet containing the purified virus was finally resuspended in THE for uses in ELISA and Western blotting.

### F. Immunological Techniques

IBDV, HCV, PRRSV and the prestained protein markers (board range, Bio-Rad) were mixed with 6× loading buffer (30 mM Tris-Cl, pH 6.8, 30% glycerol, 10% SDS, 600 mM Dithiothreitol, 0.0 12% bromophenol blue) and denatured in boiling water bath for 10 minutes. The denatured viral proteins were then resolved by sodium dodecyl sulfate-polyacrylamide gel electrophoresis (SDS-PAGE) with a vertical electrophoresis unit (Hoefer Scientific Instruments). The samples were first concentrated in a 5% stacking gel and then resolved in a 12% separating gel. Electrophoresis was performed in protein running buffer at 80 V for 2.5 hours. The gel was stained with coomassie blue or used for the Western Blot.

### 1. Western Blotting

The resolved protein bands on the SDS-PAGE gel, the bands were transferred to absolute methanol-soaked protein membrane (Immun-Blot PVDF membrane, Bio-Rad) in the vertical unit with transfer buffer at a current of 40 mA overnight.

The membrane was then rinsed with PBS and blocked in blocking agent (5% non-fat milk and 1% Tween-20 in 1×PBS) at room temperature (RT) for 15 minutes with shaking.

The blocking agent was removed and the mice anti-sera, diluted in blocking reagent, was added and incubated at RT for 2 hours with shaking. The membrane was then washed with PBS five times at 5 minutes each. After that, alkaline phosphatase (AP) conjugated goat anti-mouse IgG (Zymed, 1:50), which acted as a secondary antibody, was added to the membrane and incubated at RT for 1.5 hours. The membrane was rinsed with PBS as above. 10% 5-Bromo-4-chloro-3-indo[y] phosphate (BCIP) and 10% Nitroblue tetrazolium salt (NBT) were finally added as a substrate. Color was allowed to be developed in the dark overnight.

### 2. Enzyme-Linked Immunosorbent Assay (ELISA)

The ELISA assay was modified to measure IBCV-, HCV- and PRRSV-specific antibody in vaccinated mice. Purified IBDV, HCV or PRRSV was firstly diluted at 1:100 using coating buffer, PBSN (15 mM Na₂CO₃, 35 mM NaHCO₃ and 0.05% NaN₃, pH 9.6*in 1×PBS). 100 μl of diluted virus was added to each well of a 96-wells ELISA plate at 4° C. overnight. The plate was washed with PBST (0.05% Tween-20 IN 1×PBS) three times at 5 minutes each. The non-specific binding sites were then blocked by PBS with N Bovine Serum Albumin (BSA, USB Inc.) at 37° C. for 2 hours. 100 μl diluted mouse serum (1:50) was added to the antigen-coated wells in duplication as primary antibody and incubated at 37° C. for 1 hour. The plate was then washed with PBS as above. After then, HRP-conjugated goat anti-mouse IgG (Zymed, 1:5000) was added as secondary antibody and incubated at 37° C. for 1 hour. The plate was then washed with PBST as above. 100 μl substrate TMB (Zymed) was added to each well and allowed to react in the dark for 15 minutes. 100 μl stop solution (1N HCl) was finally added to stop the reaction. OD reading of ELISA plate was measured at 490 nm with microplate reader (Bio-Rad Model 550).

### Example 10

### ELISA and Western Blotting Performance

During the whole immunization scheme (Example 3), three of the mice died after vaccination or blood collection. This included one mouse from group 4 and one mouse from group 7 in demonstration 1 and one mouse from group 2 in demonstration 2.

In order to assess the ability of the DNA construct to elicit specific antibodies in an animal model (i.e., BALB/c mice), ELISA and Western blotting were performed.

### A. ELISA

ELISA was performed to investigate if there was any humoral immune response in the vaccinated mice. Specific anti-IBDV, anti-HCV or anti-PRRSV antibodies in the mice sera were tested by the modified ELISA in duplicate.

The results are shown from Table 10 to Table 30 on subsequent pages. They were summarized in the following table:

TABLE 10
The humoral immune response of mice from
groups 3 and 4 after vaccination.

| | Demonstration 1 | | Demonstration 2 | |
|---|---|---|---|---|
| | # of mice | Total | # of mice | Total |
| | producing | number | producing | number |
| | specific | of | specific | of |
| | antibodies | mice | antibodies | mice |
| Group 1 (IBC vaccine) | 1 | 5 | 3 | 5 |
| Group 2 (IBD vaccine) | 3 | 5 | 4 | 4 |
| Group 3 (HC vaccine) | 3 | 5 | 2 | 5 |
| Group 4 (HC vaccine) | 3 | 4 | 3 | 5 |
| Group 5 (PRRS | 3 | 5 | 3 | 5 |
| vaccine) | | | | |
| Group 6 (HC vaccine) | 5 | 5 | 2 | 5 |
| Group 6 (PRRS | 3 | 5 | 2 | 5 |
| vaccine) | | | | |

Although most of the mice sera showed a slight decrease in ELISA reading after the last vaccination as compared to the third vaccination, the reading was still higher than the initial value. Hence, the DNA vaccine utilized was proven to be effective.

A transient increase occurred when the ELISA reading of a particular mouse increased after the first and/or the second vaccination. However, the reading dropped to a level similar to that of Day 0 (pre-treatment) afterwards.

Among the 10 mice injected with pcDNA3.1-VP5-5.2 &VP2-3.4 (group 1), mouse 4 from demonstration I (Table 11) and mice 1, 3 and 5 from demonstration 2 (Table 12) showed an increase in IBDV-specific antibodies.

TABLE 11
IBDV specific ELISA reading of group 1 vaccinated mice

| | | |
|---|---|---|
| Group | Code of | ELISA reading |

| number | mice | Day 0 | Day 7 | Day 15 | Day 28 |
|---|---|---|---|---|---|
| 1 | 1 | 0.189 | 0.149 | 0.209 | 0.236 |
| | 2 | 0.261 | 0.171 | 0.189 | 0.281 |
| | 3 | 0.252 | 0.245 | 0.176 | 0.186 |
| | 4 | 0.137 | 0.248 | 0.358 | 0.590 |
| | 5 | 0.158 | 0.235 | 0.205 | 0.252 |

TABLE 12
IBDV specific ELISA reading of group 1 vaccinated mice

| | | |
|---|---|---|
| Group | Code of | ELISA reading |

| number | mice | Day 0 | Day 7 | Day 15 | Day 28 |
|---|---|---|---|---|---|
| 1 | 1 | 0.416 | 0.416 | 0.574 | 0.654 |
| | 2 | 0.535 | 0.505 | 0.638 | 0.549 |
| | 3 | 0.166 | 0.256 | 0.319 | 0.314 |
| | 4 | 0.251 | 0.209 | 0.337 | 0.291 |
| | 5 | 0.224 | 0.335 | 0.379 | 0.368 |

Among the 9 mice fed with E. coli cells containing 100 μg pcDNA3.1-VP5-5.2 & VP2-3.4 (group 2), mice 1, 2 and 3 from demonstration 1 (Table 13) and all mice from demonstration 2 (Table 14) showed an increase of IBDV-specific antibody after vaccinating orally.

TABLE 13
IBDV specific ELISA reading of group 2 vaccinated mice

| | | |
|---|---|---|
| Group | Code of | ELISA reading |

| number | mice | Day 0 | Day 7 | Day 15 | Day 28 |
|---|---|---|---|---|---|
| 2 | 1 | 0.420 | 0.626 | 0.654 | 0.638 |
| | 2 | 0.389 | 0.530 | 0.660 | 0.714 |
| | 3 | 0.378 | 0.526 | 0.659 | 0.579 |
| | 4 | 0.614 | 0.495 | 0.547 | 0.601 |
| | 5 | 0.638 | 0.593 | 0.628 | 0.600 |

TABLE 14
IBDV specific ELISA reading of group 2 vaccinated mice

| | | |
|---|---|---|
| Group | Code of | ELISA reading |

| number | mice | Day 0 | Day 7 | Day 15 | Day 28 |
|---|---|---|---|---|---|
| 2 | 1 | 0.379 | 0.492 | 0.294 | 0.836 |
| | 2 | 0.434 | 0.640 | 0.569 | 0.885 |
| | 3 | 0.472 | 0.277 | 0.527 | 0.626 |
| | 4 | 0.406 | 0.382 | 0.410 | 0.747 |
| | 5* | 0.440 | 0.464 | / | / |

*Mouse number 5 was dead after collecting blood at day 7.

Among the 10 mice injected with pHCV2.5 (group 3), mice 2,3 and 4 from demonstration I (Table 15) showed an increase in ELISA reading. Meanwhile, mice 4 and 5 from demonstration 2 (Table 16) showed a transient increase in HCV-specific antibodies.

TABLE 15
HCV specific ELISA reading of group 3 vaccinated mice

| | | |
|---|---|---|
| Group | Code of | ELISA reading |

| number | mice | Day 0 | Day 7 | Day 15 | Day 28 |
|---|---|---|---|---|---|
| 3 | 1 | 0.837 | 0.740 | 0.916 | 0.792 |
| | 2 | 0.326 | 0.555 | 0.858 | 0.661 |
| | 3 | 0.404 | 0.993 | 1.102 | 0.934 |
| | 4 | 0.727 | 0.712 | 0.956 | 0.749 |
| | 5 | 0.684 | 0.846 | 0.758 | 0.518 |

TABLE 16
HCV specific ELISA reading of group 3 vaccinated mice

| | | |
|---|---|---|
| Group | Code of | ELISA reading |

| number | mice | Day 0 | Day 7 | Day 15 | Day 28 |
|---|---|---|---|---|---|
| 3 | 1 | 0.772 | 0.621 | 0.484 | 0.579 |
| | 2 | 0.690 | 0.527 | 0.482 | 0.741 |
| | 3 | 0.659 | 0.513 | 0.549 | 0.673 |
| | 4 | 0.746 | 0.543 | 0.827 | 0.627 |
| | 5 | 0.406 | 0.598 | 0.480 | 0.529 |

Among the 9 mice fed with E. coli cells containing 100 μg pHCV2.5 (group 4), mice 1, 2 and 5 from demonstration 1 (Table 17) and mice 1, 3 and 5 from demonstration 2 (Table 18) showed an increase in HCV-specific antibodies, though mouse 5 from the demonstration 2 only showed a transient increase.

TABLE 17
IBDV specific ELISA reading of group 4 vaccinated mice

| | | |
|---|---|---|
| Group | Code of | ELISA reading |

| number | mice | Day 0 | Day 7 | Day 15 | Day 28 |
|---|---|---|---|---|---|
| 4 | 1 | 0.743 | 0.899 | 0.989 | 0.948 |
| | 2 | 0.809 | 0.936 | 0.965 | 1.097 |
| | 3* | / | / | / | / |
| | 4 | 1.002 | 1.135 | 1.102 | 1.115 |
| | 5 | 0.785 | 0.853 | 0.991 | 1.009 |

*Mouse number 3 was dead before the start of the vaccination scheme.

TABLE 18
HCV specific ELISA reading of group 4 vaccinated mice

| | | |
|---|---|---|
| Group | Code of | ELISA reading |

| number | mice | Day 0 | Day 7 | Day 15 | Day 28 |
|---|---|---|---|---|---|
| 4 | 1 | 0.660 | 0.802 | 0.644 | 0.869 |
| | 2 | 0.924 | 0.887 | 0.801 | 0.711 |
| | 3 | 0.523 | 1.088 | 0.910 | 0.790 |
| | 4 | 0.849 | 0.870 | 0.735 | 0.880 |
| | 5 | 0.648 | 0.981 | 0.855 | 0.713 |

Among the 10 mice injected with pcDNA3. I-ORF5 (group 5), mice 3, 4 and 5 from demonstration 1 (Table 19) and mice 1, 2 and 5 from demonstration 2 (Table 20) showed an increase in PRRSV-specific antibody level after vaccination.

TABLE 19
PRRSV specific ELISA reading of group 5 vaccinated mice

| | | |
|---|---|---|
| Group | Code of | ELISA reading |

| number | mice | Day 0 | Day 7 | Day 15 | Day 28 |
|---|---|---|---|---|---|
| 5 | 1 | 0.513 | 0.424 | 0.390 | 0.419 |
| | 2 | 0.483 | 0.358 | 0.681 | 0.500 |
| | 3 | 0.278 | 0.425 | 0.572 | 0.514 |
| | 4 | 0.220 | 0.451 | 0.525 | 0.433 |
| | 5 | 0.396 | 0.473 | 0.749 | 0.551 |

TABLE 20
PRRSV specific ELISA reading of group 5 vaccinated mice

| | | |
|---|---|---|
| Group | Code of | ELISA reading |

| number | mice | Day 0 | Day 7 | Day 15 | Day 28 |
|---|---|---|---|---|---|
| 5 | 1 | 0.431 | 0.354 | 0.326 | 0.525 |
| | 2 | 0.300 | 0.331 | 0.347 | 0.466 |
| | 3 | 0.371 | 0.245 | 0.425 | 04.62 |
| | 4 | 0.253 | 0.353 | 0.319 | 0.282 |
| | 5 | 0.285 | 0.316 | 0.404 | 0.701 |

Among the 10 mice injected with a combined vaccine of pHCV2.5 and pcDNA3.1-ORFS (group 6), all mice from demonstration 1 (Table 21) and mice 4 and 5 from demonstration 2 (Table 22) showed an increase in HCV-specific antibodies. In the meantime, mice 3, 4 and 5 from demonstration 1 (Table 23) and mice 2 and 4 from demonstration 2 (Table 24) showed an increase in PRRSV-specific antibodies.

TABLE 21
HCV specific ELISA reading of group 6 vaccinated mice

| | | |
|---|---|---|
| Group | Code of | ELISA reading |

| number | mice | Day 0 | Day 7 | Day 15 | Day 28 |
|---|---|---|---|---|---|
| 6 | 1 | 0.304 | 0.915 | 0.755 | 1.000 |
| | 2 | 0.340 | 0.939 | 0.851 | 0.611 |
| | 3 | 0.267 | 0.814 | 0.922 | 0.737 |
| | 4 | 0.197 | 0.601 | 0.764 | 0.688 |
| | 5 | 0.424 | 0.951 | 0.932 | 0.848 |

TABLE 22
HCV specific ELISA reading of group 6 vaccinated mice

| | | |
|---|---|---|
| Group | Code of | ELISA reading |

| number | mice | Day 0 | Day 7 | Day 15 | Day 28 |
|---|---|---|---|---|---|
| 6 | 1 | 0.711 | 0.621 | 0.659 | 0.741 |
| | 2 | 0.733 | 0.553 | 0.716 | 0.737 |
| | 3 | 0.897 | 0.453 | 0.834 | 0.831 |
| | 4 | 0.862 | 0.556 | 0.729 | 0.999 |
| | 5 | 0.626 | 0.441 | 0.819 | 1.082 |

TABLE 23
PRRSV specific ELISA reading of group 6 vaccinated mice

| | | |
|---|---|---|
| Group | Code of | ELISA reading |

| number | mice | Day 0 | Day 7 | Day 15 | Day 28 |
|---|---|---|---|---|---|
| 6 | 1 | 0.380 | 0.420 | 0.395 | 0.380 |
| | 2 | 0.355 | 0.390 | 0.525 | 0.405 |
| | 3 | 0.395 | 0.365 | 0.470 | 0.760 |
| | 4 | 0.455 | 0.365 | 0.370 | 0.720 |
| | 5 | 0.510 | 0.350 | 0.365 | 0.685 |

TABLE 24
PRRSV specific ELISA reading of group 6 vaccinated mice

| | | |
|---|---|---|
| Group | Code of | ELISA reading |

| number | mice | Day 0 | Day 7 | Day 15 | Day 28 |
|---|---|---|---|---|---|
| 6 | 1 | 0.460 | 0.595 | 0.385 | 0.425 |
| | 2 | 0.390 | 0.430 | 0.375 | 0.885 |
| | 3 | 0.450 | 0.430 | 0.385 | 0.380 |
| | 4 | 0.415 | 0.440 | 0.565 | 0.400 |
| | 5 | 0.435 | 0.370 | 0.365 | 0.385 |

None of the mice in the negative control groups (Table 25 to Table 30) showed any increase in the anti-HCV or anti-PRRSV antibody levels.

TABLE 25
IBDV specific ELISA reading of mice in the control group

| | | |
|---|---|---|
| Group | Code of | ELISA reading |

| number | mice | Day 0 | Day 7 | Day 15 | Day 28 |
|---|---|---|---|---|---|
| Control | 1 | 0.434 | 0.445 | 0.319 | 0.254 |
| group* | 2 | 0.389 | 0.422 | 0.308 | 0.260 |
| | 3 | 0.284 | 0.320 | 0.239 | 0.222 |
| | 4 | 0.252 | 0.312 | 0.302 | 0.202 |

*Mouse number 5 was dead before the start of the vaccination scheme.

TABLE 26
IBDV specific ELISA reading of mice in the control group

| | | |
|---|---|---|
| Group | Code of | ELISA reading |

| number | mice | Day 0 | Day 7 | Day 15 | Day 28 |
|---|---|---|---|---|---|
| Control | 1 | 0.240 | 0.206 | 0.215 | 0.218 |
| group* | 2 | 0.417 | 0.417 | 0.316 | 0.284 |
| | 3 | 0.266 | 0.282 | 0.353 | 0.205 |
| | 4 | 0.333 | 0.254 | 0.206 | 0.222 |
| | 5 | 0.242 | 0.328 | 0.247 | 0.212 |

TABLE 27
HCV specific ELISA reading of mice in the control group

| | | |
|---|---|---|
| Group | Code of | ELISA reading |

| number | mice | Day 0 | Day 7 | Day 15 | Day 28 |
|---|---|---|---|---|---|
| Control | 1 | 0.686 | 0.626 | 0.549 | 0.515 |
| group* | 2 | 0.582 | 0.593 | 0.604 | 0.568 |
| | 3 | 0.533 | 0.530 | 0.453 | 0.489 |
| | 4 | 0.500 | 0.593 | 0.586 | 0.507 |

*Mouse number 5 was dead before the start of the vaccination scheme.

TABLE 28
HCV specific ELISA reading of mice in the control group

| | | |
|---|---|---|
| Group | Code of | ELISA reading |

| number | mice | Day 0 | Day 7 | Day 15 | Day 28 |
|---|---|---|---|---|---|
| Control | 1 | 0.647 | 0.480 | 0.650 | 0.599 |
| group | 2 | 0.844 | 0.904 | 0.609 | 0.543 |
| | 3 | 0.572 | 0.519 | 0.500 | 0.435 |
| | 4 | 0.772 | 0.710 | 0.782 | 0.560 |
| | 5 | 0.508 | 0.625 | 0.587 | 0.461 |

TABLE 29
PRRSV specific ELISA reading of mice in the control group

| | | |
|---|---|---|
| Group | Code of | ELISA reading |

| number | mice | Day 0 | Day 7 | Day 15 | Day 28 |
|---|---|---|---|---|---|
| Control | 1 | 0.243 | 0.242 | 0.180 | 0.203 |
| group* | 2 | 0.152 | 0.235 | 0.203 | 0.230 |
| | 3 | 0.185 | 0.201 | 0.169 | 0.204 |
| | 4 | 0.188 | 0.253 | 0.205 | 0.202 |

*Mouse number 5 was dead before the start of the vaccination scheme.

TABLE 30
PRRSV specific ELISA reading of mice in the control group

| | | |
|---|---|---|
| Group | Code of | ELISA reading |

| number | mice | Day 0 | Day 7 | Day 15 | Day 28 |
|---|---|---|---|---|---|
| Control | 1 | 0.218 | 0.197 | 0.233 | 0.235 |
| group | 2 | 0.278 | 0.358 | 0.196 | 0.159 |
| | 3 | 0.218 | 0.249 | 0.289 | 0.169 |
| | 4 | 0.271 | 0.171 | 0.193 | 0.178 |
| | 5 | 0.217 | 0.234 | 0.241 | 0.209 |

### B. Western Blotting

Western blot analysis showed whether the viral envelope protein has been expressed. Moreover, it also demonstrated the specificity of mice-antisera to the viral protein.

The viral proteins were heat-treated at 100° C. for 10 minutes and resolved in the SDS-PAGE, so the viral proteins were denatured. Therefore, only linear epitopes, but not conformation dependent epitopes, could be detected on the blotting membrane.

All the mice sera (collected on day 28) in a group were pooled together and diluted with blocking agent for the Western Blot analysis.

All mice sera in demonstrations 1 and 2 showed a positive result in the Western blot analysis, although there was variation in intensity of the bands.

There was no banding pattern for control mice sera pooled from demonstration 1 or pooled from demonstration 2 against IBDV, HCV and PRRSV protein. In the other words, there were no specific anti-IBDV, anti-HCV or anti-PRRSV antibodies produced.

In the Western blot of group 1 and 2 mice sera, wherein pooled sera from demonstration 1 and pooled sera from demonstration 2 were tested, there was a band between 36.4 kD and 486 kD. It corresponded to VP 2 of IBDV. The viral proteins were run in duplicate for each test.

In the Western blot of group 3 mice sera, there were two bands between 48.6 kD and 96 kD. The viral proteins were run in duplicate for each test. One band was closer to 48.6 kD in the pooled mice anti-sera from demonstration 1 and represented the E₂ protein of HCV (55 kD). There was an additional band between 29.8 kD and 36.4 kD. The band corresponded to E 1 of HCV (33 kD). Meanwhile, one band was closer to 94 kD in the pooled mice anti-sera from demonstration 2 which corresponded to E₁-E₂ heterodimer of HCV (75 kD). Glycoprotein E0 has been described as a 44 kD to 48 kD protein and was observed slightly lower than 48.6 kD on the blotting membrane.

The result of the Western blot of group 4 mice sera was similar to that in group 3.

In case of group 5 mice sera, there was a band just below 29.8 kD. It corresponded to GP5 of PRRSV. In the demonstration 1 test, the strong band was between 29.8 kD and 36.4 kD. Although typical GP5 was 24.5 kD-26 kD, this strong band should represent GP5 since pcDNA3.1-ORFS was injected. The difference may due to incomplete denaturation of the protein and resolving ability of the polyacrylamide gel.

The Western blot of group 6 mice sera showed, on the lane with HCV glycoprotein in the test of the demonstration I anti-sera, a band just higher than 94 kD that represented E₂-E₂ homodimer of HCV (100 kD). Glycoprotein EO (44 kD to 48 kD) was observed slightly lower than 48.6 kD on the blotting membrane. Moreover, there was a band between 29.8 kD and 36.4 kD. The band corresponded to E1 of HCV (33 kD). In the test of the demonstration 2 antisera, on the lane with HCV glycoprotein, there was a band close to 94 kD. It corresponded to E₁-E₂ heterodimer of HCV (75 kD).

On the lane with PRRSV glycoprotein, there was a band just below 29.8 kD. It corresponded to GP5 of PRRSV in both the demonstration I and demonstration 2 anti-sera tests. Above the GP5 band, there were several bands. They may be the incomplete denatured GP5 or the disulfide-linked M & GP5 heterodimer.

### C. Results

In the modified ELISA analysis, although ELISA plates were blocked by the blocking agent to avoid non-specific binding, non-specific background staining was still seen in some samples wells. For example, in group 3 mice in demonstration 1, the initial reading of number 1, 4 and 5 showed a deviated value from that of number 2 and 3 which was run on another plate. Hence, the trend of ELISA reading over time has also been taken into consideration for the evaluation of specific antibodies production.

In ELISA analysis, 5 out of 10 mice injected with pcDNA3.I-VP5-5.2&VP2-3.4 have been shown to produce specific antibodies against IBDV. At the same time, 7 out of 9 mice fed with E. coli containing pcDNA3.1-VP5-5.2&VP2-3.4 have shown to produce specific antibodies against IBDV.

Five out of ten mice injected with pHCV2.5 have been shown to produce specific antibodies against HCV. At the same time, 6 out of 9 mice fed with E. coli containing pHCV2.5 have shown to produce specific antibodies against HCV.

The above results indicate that the DNA vaccine administered through the two routes (intramuscular injection and feeding) was effective in triggering specific antibodies against the virus. The result was further confirmed by Western blot analysis.

In ELISA analysis, 5 out of 10 mice injected with pHCV2.5 have been shown to produce specific antibodies against HCV. At the same time, 6 out of 10 mice injected with pcDNA3.1-ORF5 have been shown to produce specific antibodies against PRRSV.

In group 6, the mice were injected with a combined DNA vaccine (pHCV2.5 and pcDNA3,1-ORFS); 7 out of 10 mice have been shown to produce specific antibodies against HCV. At the same time, 5 out of 10 mice have been shown to produce specific antibodies against PRRSV. In addition, 4 mice in this group have been shown to produce specific antibodies against HCV and PRRSV simultaneously.

The above results indicate that the combined DNA vaccine administered through intramuscular injection was effective in triggering specific antibodies against both viruses (HCV and PRRSV). The result was further confirmed by Western blot analysis.

The antibody reaction against each virus using a combined immunization was rather equivalent in quality and intensity to that obtained with a single immunization, considering the mouse to mouse variability. This indicates that the mouse immune system could accommodate multiple antigens and open the way for multipotent DNA vaccine preparations for pigs.

There are at least three conformation dependent and one linear (conformation independent) neutralizing epitopes in IBDV. Two conformation dependent neutralizing epitopes located in the central region of VP2 while the linear neutralizing epitope was located in the C-terminal of VP3 (Yamaguchi, T., et al., 1996, Virol 223:219-223). Although Western blots can only detect linear antigens, VP2 was detected on the membrane. It may due to the incomplete denaturation of VP2 that made it detectable.

In the Western Blot analysis, homodimers and heterodimers of HCV have been detected. Dimerization of HCV protein maybe important for authentic antigen presentation to the host's immune system and the induction of a stable, long-term immunity (Konig, M et al., 1995, J Virol 69:5479-86). In addition, since the viral proteins were separated in non-denaturing conditions, an E₁-E₂ heterodimer of HCV that was linked by intermolecular disulfide bridges could be detected. Intramolecular disulfide bonds coated in the N-terminal half of E₂ have been shown to be important for antigen recognition by specific antibodies. In order to demonstrate monomeric E₁ and E₂, the proteins should be separated under reducing conditions (e.g. in the presence of 2-mecaptoethanol).

Three different forms of glycoproteins E₂ were found in HCV; they were E₂ monomers, E₂ homodimers and E1-E₂ heterodimers. E₁-E₂ heterodimers were preferentially formed after introducing pHCV2.5 that expressed both E₁ and E₂. The appearance of doublets or triplets of these forms may be due to different E₂ protein backbones and to different glycosylation. Moreover, alternative processing at the carboxy terminus generated E₂ molecules with different apparent molecular weights.

In Western blot analysis of PR RSV-specific antibodies, there were a number of bands. They may be due to the following reasons: firstly, the PRRSV protein was heat-treated at 100° C. for 10 minutes before loading onto the SDS-PAGE. Hence, three types of GPS may appear on the gel: i. Totally denatured, ii. Partially denatured, iii. Partially degraded.

Secondly, at around 26.4 kD and 48.6 kD, there was a clear band that may correspond to the disulfide-linked M-GP5 heterodimer. Cysteine residues located at the N-terminal ectodomains of both envelope proteins were probably involved in the formation of an intermolecular disulfide bridge. Hence, the presence of anti-GP5 antibody could detect its presence.

There were at least two types of neutralizing antigenic determinants associated with the GP5 of PRRSV. Among these determinants, some were conformation dependence and some were linear. Therefore, Western blot analysis could still detect the presence of anti-GP5. In addition, it appeared that glycosylation was not necessarily associated with the neutralizing epitopes.

### Example 11

### Modifications to Vaccine Compositions and Methods

It is contemplated that certain modifications to the vaccine compositions and methods demonstrated herein may provide additional aspects of the present invention.

Firstly, it is contemplated that a counterpart to each group should be included for comparison. For example, besides feeding mice with E. coli containing the plasmid (potential DNA vaccine), an additional group of mice should be fed with naked plasmid DNA. Besides injecting mice with naked plasmid DNA, an additional group of mice should be included and injected with E. coli containing the plasmid. Moreover, instead of injecting or feeding nothing to the mice in the control group, they could be injected with PBS or vector (pcDNA3.1) alongside the vaccination scheme.

Secondly, it is contemplated that the production of IgA and cytokines could be confirmed by analyzing frozen small intestinal tissue sections of the vaccinated mice containing the most Peyer's patches. It has been said that oral immunization can elicit the production of both systemic and mucosal antibodies (Gallichan W S, Rosenthal K L. 1995, Vaccine. 13:1589-1595).

Thirdly, although the DNA vaccine (pcDNA3.1-VP505.2&VP2-3.4, pHCV2.5 and pcDNA3. 1-ORFS) can elicit the production of specific antibodies, as previously demonstrated, it is contemplated that a DNA vaccine may be able to protect the animal against the disease. To further demonstrate the efficacy of the DNA vaccine, the neutralizing ability of the specific antibodies can also be tested by virus neutralization assay.

Fourthly, it has been said that immunization with DNA vaccine can elicit both humoral and cellular immunity (Ulmer, J. B., et al., 1996, Immunol 89: 59-67; Ulmer, J. B., et al., 1996, Cur Opin Immunol 8:531-536). However, only the humoral immune response was measured after each vaccination. It is contemplated that T lymphocytes, which play an important role in fighting against the virus, may be stimulated by the vaccine and compositions of the present invention to produce an immune response. It is further contemplated that the quantity and the role of antigen specific cytotoxic and helper T lymphocytes in fighting disease, after vaccination using the compositions and methods of the present invention, may be demonstrated by flow cytometric analysis.

Fifthly, it is contemplated that an anesthetized animal may be easier to vaccinate using the compositions and methods of the present invention. For example, during injection of mice with a DNA vaccine, a vaccinated animal may contract their muscles and squeeze the vaccine solution (e.g., a DNA vaccine) out when they were awake.

Sixthly, it is contemplated that in order to enhance or boost the immune response of an animal (e.g. mice), one or more additional agents may be used in combination with any of the vaccine methods and compositions described herein. Such agents may include, but are not limited to, one or more chemical (e.g., complete Freund's adjuvant) or genetic (e.g., vector expressing cytokines) adjuvants. In a non-limiting example, co-inoculation of both an immunogenic DNA vaccine (e,g. a plasmid) and a genetic adjuvant (e.g. another plasmid) may result in an augmentation of an antigen specific humoral and/or cell mediated immune response.

### Example 12

### Demonstration of Isolation and Characterization of a Porcine Pathogenic Virus

Described herein this example provides for methods for identifying, isolating and characterizing a pathogen, specifically Guangzhou porcine reproductive and respiratory virus. Specifically described is a method to detect the presence of porcine reproductive and respiratory syndrome virus (PRRSV) in tissue samples of infected pigs, based on reverse transcription of the viral RNA coupled to DNA amplification by polymerase chain reaction (RT-PCR). Tissue samples (lung, muscles) were collected from infected pigs from different pig farms in Guangzhou. AV Guangzhou field isolates described herein have a close similarity with virions of North American strains through the RT-PCR amplification, sequencing analysis, and Western immunoblotting analysis. As would be understood by one of skill in the art, such methods may be applied in identifying, isolating and characterizing other pathogens in the practice of the present invention.

### Identification of PRRSV as a New Pathogen

In the late 1980's, a mysterious disease broke out in pig farms throughout the world, characterized by severe reproductive failure in sows of any parity and respiratory problems for pigs of all ages (Mardassi et al., Can J Vet Res 58, 55-64; Mardassi et al., 1994. J Clinical Microbiology. September 2197-2203). The disease was first reported in North American in 1987, and the etiological agent was first isolated in 1990 in Europe and then in 1992 in the USA (Suarez et al., 1994 Arch Virol. 135, 89-99). Both strains were structurally related but antigenically distinct. The disease was finally given the name Porcine Reproductive and Respiratory Syndrome disease, caused by a fastidious virus-Porcine Reproductive and Respiratory Syndrome Virus (PRRSV).

### Characterization of PRRSV

Molecular characterizations have shown that the viral genome consists of a positive single-stranded RNA molecule with an approximate genome size of 15 kb. The genome contains 8 open reading frames (ORFs) with ORFIa and ORFIb (at the 5′ end) representing nearly 75% of the viral genome. It encodes for the proteins with the function of polymerase and replicase activities. The other six structural proteins, ORFs2 to 7, are located at the 3′ end of the genome (Conzelmann, K. K. et al., 1993, Virol 193: 329-339). The spherical, envelope PRRSV has been described with morphological and morphogenetical similarity to members of the arterivirus group, including equine arteritis virus (EAV) and lactafe dehydrogenase-elevating virus of mice (LDV). Virus replication has failed in many different kinds of primary and established cell lines, but it does replicate in Porcine Alveolar Macrophages (PAM) and MARC-145 derived from MA-104 monkey cell (Dea et al., Ultrastructural characteristics and morphogenesis of Porcine Reproductive and Respiratory Syndrome Vrus propagated in the highly permissive Marc-145 cell clone. Plenum Press, New York 1995).

### Isolation and Characterization of the Sub-Strains of PRRSV

In brief, multiplex PCR utilizing different sets of oligonucleotide primers was used to differentiate between North American and European isolates among the field isolated samples collected from Guangzhou. Using purifed PRRS virion from the infected cell cultures as the starting material, molecular cDNA cloning and sequencing was also performed. The morphological and physicochemical properties of the field isolate of PRRSV were characterized as well.

### 1. Cells and Virus Isolation

A total of 3 Guangzhou field isolates of PRRSV were recovered from clarified lung tissues and muscles of dyspneic pigs and were propagated on MARC-145, a PRRSV permissive cell line (Department of Veterinary Medicine, South China Agricultural University, Guangzhou, 510642, China). The MARC-145 cells were cultivated in Minimum Essential Medium (MEM) supplemented with 10% gamma-irradiated fetal bovine serum (FBS) and 10% Tryptose Phosphate Broth (TPB) and 1% antibiotic-Antimycotic (Life Technologies, GIBCOBRL®). The representative US isolate of PRRSV, NVSL, was isolated from commercially available modified-live vaccine (Animal and Plant Health Inspection Services, National Veterinary Services Laboratories. Ames, Iowa). Aliquots of 1.5 ml of clarified tissue samples were inoculated into the cell monolayer of the 75 cm² culture flask with culture medium containing no fetal bovine serum (FBS). Infected cell culture was monitored daily for the appearances of cytopathic effect (CPE).

For all tissue sample homogenates, no CPE was observed during the first six passages in the MARC-145 cell culture. CPE was observed after the seventh passage beginning with the characteristics of rounding off; cell enlargement and rounded cells with many vacuoles appearing after 24 hours post-inoculation with the AV field isolates of PRRSV; syncytia of the infected cells was observed; the infected cells started to aggregate after 3-4 days post-inoculation. Finally the infected cell sloughed off from the flask. These CPE was also seen on positive control NVSL infected cells throughout the experiment.

### 2. Virus Purification

Stock viruses were produced by at least seven successive passages in MARC-145 cells. The viruses were harvested by freezing and thawing the infected cells three times at −80° C. and 37° C. respectively. The cellular debris was removed by centrifugation at 4000×g for 20 min at 4° C. The extracellular virus in the clarified supernatant fluids was first pelleted for 3 hours at 75,000×g (21,000 rpm) in a Beckman SW40Ti rotor. The pellet was resuspended in 1/100 original volume of THE buffer. Concentrated virus was then purified through a 30%-50% (w/v) sucrose gradient in a Beckman SW55Ti rotor at 110,000×g (35,000 rpm) for 16 hours.

### 3. RNA Preparation

Viral RNA was extracted directly from 500 μl of the supernatant from virus infected MARC-145 cells by using 500 μl TRIzol® reagent (Life Technologies Inc., Carlsbad, Calif., USA) and following the manufacturer's protocol.

4. cDNA Synthesis

cDNA synthesis was done according to the Superscript™ Preamplification System for First Strand cDNA Synthesis (Life Technologies, GIBCO BRL).

### 5. Primer Design

Several sets of primers were designed based on both North American (VR-2332) and European (LV) genome sequences within ORFIb encoding the polymerase protein in order to perform a rapid multiplex PCR assay. PCR primers were designed on the basis of ORFIb due to their conservation among arteriviruses. One set of internal primers was designed from Leiystad virus (LV) genome sequences, while the other set were type-specific internal primers and type-common primers for multiplex or nested multiplex PCR. Primers were designed after sequencing a portion of ORFIb from two North American strains of PRRS virus: Minnesota MNIb and Quebec LHVA-93.3 isolates (Gilbert et al., 1997, J. Clinical Mcrobiology January 35(1), 264-267). Details of the oligonucleotide primers for nested multiplex PCR were shown in Table 31.

TABLE 31
Oligonucleotide primers for PCR amplification and
typing of PRRSV

| | Size of PCR | Type |
|---|---|---|
| Primer Sequence (5′ to 3′) | product (bp) | Detected |
| Nested multiplex primers | 186 | European |
| U1 GTATGAACTTGCAGGATG | | |
| (SEQ ID NO: 17) | | |
| D1 GCCGACAATACCATGTGCTG | | |
| (SEQ ID NO: 18) | | |
| U2 GGCGCAGTGACTAAGAGA | 107 | North |
| (SEQ ID NO: 19) | | American |
| D2 GTAACTGAACACCATATGCTG | | |
| (SEQ ID NO: 20) | | |
| External primers | 255 | Common |
| EU CCTCCTGTATGAACTTGC | | |
| (SEQ ID NO: 21) | | |
| ED AGGTCCTCGAACTTGAGCTG | | |
| (SEQ ID NO: 22) | | |

### 6. Nested Multiplex PCR for PRRSV Typing of Samples D2, D3 and AV

For the first round of PCR, 2.5 μl of cDNA was added into the mixture containing 10×PCR buffer, 25 mM MgCl₂, 10 mM dNTPs mix, 12.5 μM primers EU and ED and 5 units of Taq DNA polymerase (Life Technologies, GIBCO, BRL) in a total volume of 25 μl. After denaturation at 94° C. for 3 min, the reactions were cycled 4 times at 94° C. for 20 sec, 42° C. for 1 min, and 72° C. for 1 min; and then 40 times at 94° C. for 20 sec, 47° C. for 1 min, and 72° C. for 1 min, with a final extension step of 72° C. for 5 min.

For nested PCR, 2.5 μl of the template (PCR product in first round PCR) was added into a reaction mixture containing 12.5 μM primers U1 and D1, and 12.5 μM primers U2 and D2. After denaturation at 94° C. for 3 min, the reactions were cycled 35 times at 94° C. for 20 sec, 47 C for 30 sec and 72° C. for 30 sec with a final extension step of 72° C. for 15 min The amplified products were detected by electrophoresing 5 μl aliquots through 1.5% agarose gel and stained with ethidium bromide, the gels were photographed under UV illumination.

No amplification was observed for the European strains, thus confirming the specificity of the internal primer pair U2 and D2 for the North American isolates and its use for the differentiation between North American and European isolates of PRRSV.

### 7. Sequencing Determination/Analysis

The RT-PCR amplified products were purified from a 1.5% agarose gel with Geneclean® II Nucleic acid Purification Kit (QBIOgene). The genomic region was sequenced on both strands using U2 and D2 primers in an Automated Laser Fluorescent DNA sequencer (PERKIN ELMER, ABI Prism™ 310 Genetic Analyzer). Computer analysis of the nucleotide and sequences was performed using the sequencing analysis 3.4 program as well as MAC DNAsis Version.2.4 software. Nucleotide homologies were also calculated using the MAC DNAsis Version 3.6 program and Geneworks Version 2.2 program (Intelligenetics, Inc., Mountain View, Calif., USA).

The obtained nucleotide sequences of the ORFIb of the 3 field PRRSV isolates were compared to the published sequences of a reference US strain (ATCC VR-2332) and a reference European strain (LV) as well as the reference NVSL. Table 32 shows no nucleotide substitutions, deletions or insertions among the 3 field PRRSV isolates.

TABLE 32
Comparison of the nucleotide sequences of the
polymerase (ORF1b) gene of the field isolates
PRRSV with the sequences of the reference NVSL,
European (LV) as well as the US (VR-2332)

| | |
|---|---|
| AV-ORFIb | GGCCTGTCGT CCGGCGACCC GATCACCTCT GTGTCTAACA |
| | CCATTTACAG TTTGGTGATC TACGCAC-AG CATATGGTGT |
| | TCAGTTACAT (SEQ ID NO: 23) |
| D2-ORFIb | GGCCTGTCGT CTGGCGACCC GATCACCTCT GTGTCTAACA |
| | CCATTTATAG TTTGGTGATA TATGCAC-AG CATATGGTGT |
| | TCAGTTACA-..........(SEQ ID NO: 24) |
| D3-ORFIb | GGCCTGTCGT CCGGCGACCC GATCAGCTCT GTGTCTAACA |
| | CCATTTACAG TTTGGTGATC TACGCAC-AG CATATGGTGT |
| | TCAGTTACAT ..........(SEQ ID NO: 25) |
| LV-ORFIb | G-TTTTCAT AAAAAAAGGT TAGAAAATAA AAATAAAATC |
| | TCCATAGCTG GTGGTTATTT TAAAAGTTAG AATGAGACTA |
| | TAAGCAATGG ..........(SEQ ID NO: 26) |
| NVSL- | GGCCTGTCGT CCGGCGACCC GATCACCTCT GTGTCTAACA |
| ORFIb | CCATTTACAG TTTGGTGATC TACGCAC-AG CATATGGTGT |
| | TCAGTTACAT ..........(SEQ ID NO: 27) |
| VR2332- | GGCCTGTCGT CTGGCGACCC GATCACCTCT GTGTCTAACA |
| ORFIb | CCATTTACAG TTTGGTGATC TACGCAC-AG CATATGGTGC |
| | TCAGTTACTT ..........(SEQ ID NO: 28) |

Phylogenetic tree analysis based on the nucleotide sequences on the polymerase gene of the filed isolates as well as the reference NVSL, European (LV) and US (VR 2332) isolates was performed. A 17.5% nucleotide homology was observed between the LV-ORF1b strain and the D2-ORFIb, VR2332-ORFIb, NVSL-ORFIb, D3-ORFIb and AV-ORF1b strains; a 95.1% nucleotide homology was observed between the D2-ORFIb strain and the VR2332-ORFIb, NVSL-ORFIb, D3-ORFIb and AV-ORFIb strains; a 95.5% nucleotide homologies of those field isolates (i.e., NVSL-ORFIb, D3-ORFIb and AV-ORFIb strains) was observed with the reference US strain (the VR2332-ORFIb strain, ATCC VR-2332); a 100% nucleotide homology was observed between the NVSL-ORFIb strain and the D3-ORF1b and AV-ORFIb strains; and a 100% nucleotide homology was observed between the D3-ORFIb and AV-ORF 1b strains.

The sequencing data of the PRRSV field isolates were used to perform phylogenic analysis by Unweighted Pair Group Method with Arithmetic Mean (Geneworks Version 2.2). Accordingly, the field PRRSV isolates were grouped in the North American genotype (the NVSL-1b, Av-Lb, D3-1b, VR-23321b and D2-OREIb strains) distinct from the reference European LV strain (LV-ORF1b).

Thus shown herein, RT-PCR can be used for the detection of several RNA viruses. This technique was applied in the present example in order to characterize the strains of the field isolates collected from Guangzhou, so as to confirm the transmission of the new porcine virus from either North America or Europe to Mainland China. It has been indicated that it is a suitable diagnostic procedure not only for detection but also for differentiation between North American and European strains of PRRSV (Mardassi et al., Can J Vet Res 58, 55-64; Mardassi et al., September 1994. J Clinical Microbiology. 2197-2203; Meulenberg et al., 1993, Virology 192, 62-74) showed that most of the PRRSV isolates were able to cultivate in PAM (Porcine Alveolar Macrophages) cells from lung homogenates that supported virus replication. Until recently, propagation of North American PRRSV isolates was achieved in the MARC-145 cells highly permissive cell clones of PRRSV derived from the MA-104 cell line (Kim et al., 1993, Arch Virol 133, 477-483). In the present example, virus isolation in MARC-145 was successful; it was found to be permissive to the Guangzhou PRRSV field isolates.

### 8. Determination of Ultra-Structural Characteristics

Ultracentrifugation of concentrated extracellular virus yielded an opalescent band at the bottom of the tube corresponding to 50% sucrose density gradient. Viruses in the fractions from density gradients were spotted on formvar-carbon-coated grids by floating the grid on 30 μl aliquots of fraction from density gradients for 30 sec and then negatively stained with 3% aqueous phosphotungstic acid (PTA) pH 6.3 for 30 sec. The grids were examined on a Philips EM 208s electron microscope at different potentials after irradiating the grid with UV light for 15 min Negative stain electron micrograph of the AV and NVSL reference viral particles observed in sucrose gradient fraction of 1.34 g/ml under different potentials (90 kV and 31.5 kV). Through electron microscopic examination, this gradient fraction contained numerous spherical, enveloped viral particles 60 nm in diameter with cellular debris.

The morphological characteristics of the AV field isolates of PRRSV were also in agreement with previously described tissue culture adapted from ATCC-VR2332American isolate of the PRRSV (Murphy et al., 1992, Vet. Microbiol. 32, 101-115; Pirzadeh, B., Gagnon, C, A. and Dea, S., 1998, Can J. Vet. Res. 62, 170-177).

### 9. Viral Polypeptide Identification

Sucrose gradient purified preparations (i.e., fractions from 50% to 30%) of the field PRRSV isolates AV and the reference NVSL PRRSV were analysed by SDS-PAGE under non-reducing conditions, and viral polypeptides were deduced from Western immunoblotting experiments using commercially available homologous hyperimmune sera of pigs as the source of specific virus antibodies. Both the SDS-PAGE and Western immunoblotting assay were done according to the Fritsch, Maniatis and Sambrook, 1989, Molecular Cloning, A Laboratory Manual (Second Edition pp. 18.47-18.75).

Western immunoblotting, reference hyperimmune serum (Animal and Plant Health Inspection Service, National Veterinary Services Laboratories, Ames, Iowa), Alkaline Phosphatase (AP) labeled rabbit anti-porcine IgG (Zymed) and enzyme-substrate solution consisting of NBT and BCIP buffer solution. (Zymed, S. San Francisco, Calif., USA) were used. The relative migration distances of the bands observed in the gels correspond to approximate MW of 15 kDa, 19 kDa, 26 kDa, 45 kDa and 80 kDa. Only the 15 kDa, 19 kDa, 26 kDa and 45 kDa viral polypeptides were immunoprecipitated using the positive control antisera.

From the result of the Western immunoblotting experiments, it appears that from the bands obtained by SDS-PAGE, only those corresponding to estimated MW of 15 kDa, 19 kDa, 26 kDa and 45 kDa may in fact represent viral proteins. Other bands detected by SDS-PAGE could correspond to cellular proteins co-purified with the extracellular virions. The polypeptide patterns identified for the Guangzhou field isolate AV appear identical to those reported for the American reference isolate ATCC-VR2332 propagated in the continuous CL2621 cell line (Francki et al., 1992, Arch Virol. 2, 220-222) as well as the reference NVSL. As mentioned by the others (Plageman et al., 1992, Adv. Virus Res. 41, 91-192; Mardassi et al, 1994), these polypeptide patterns are compatible with those determined for EAV and LDV. By analogy to EAV and LDV, the 15 kDa, 19 kDa and 26 kDa polypeptides identified for PRRSV represent the major nucleocapsid protein N, the matrix protein M and the envelope protein E respectively, while the 30 kDa, 31 kDa and 45 kDa polypeptides represent minor structural protein of the viruses.

### Example 13

### Demonstration of Isolation and Characterization of a Chicken Pathogenic Virus

The example herein demonstrates the isolation and characterization of different sub-strains of chicken Infectious Bronchitis Virus (IBV) based on Si gene diversity. Five regional 113V isolates collected from different geographical region in Southern China were characterized with PCR sequencing. A pair of primer flanking the whole S-1 gene of the IBV was designed according to the published sequence data, and the expected PCR products size is 1760-base pair. The resulting PCR product was further sub-cloned into pGEM-T easy vector and subjected to sequencing. The analysis of the genetic relationship among the isolates indicates the diversity of S-1 gene of the 5 isolates in Southern China was very high. The nucleotide variation among these 5 isolates ranged from 8% to 48%, and the 5 isolates could be classified into 3 groups according to the phylogenetic tree analysis. Two conserved regions and two hyper-variable regions were also identified among these isolates. This example indicates chicken farms in different regions of China should vaccinate their chicken with their respective genotype matched vaccine strains of the particular region so as to prevent failure of vaccination. Additionally, other pathogen substrains can be identified for more effective vaccine preparation and administration in accordance with the present invention.

### Virus Collection

Field IBV isolates V1, V2, V3, V4 and V5 were obtained from Yunnan, Hunan, Hubei, Guangxi, and Guangdong provincial veterinary service stations of China.

### Virus Isolation

Each isolate was propagated and titrated in 10-day-old specific-pathogen-free (SPF) embryonated chicken eggs at 37° C. for 48 hrs. 500 microliters of allantoic fluid was collected from inoculated embryos and centrifugated at 2500×g for 5 min at 4° C. After centrifugation, the supernatant was collected; IRV genomic RNA was isolated according to manufacturer's instructions (Gibco BRL, Grand Island, N.Y.).

### RT-PCR

Viral RNA was used as a template to reverse transcribe the first strand cDNA. The superscriptase RT kit was used per manufacturer's instructions (Gibco BRL, Grand Island, NY). cDNA was synthesized from 1 μl (200 ng) viral RNA primed with random hexamers. Amplification of cDNA was performed in a volume of 25 μl that included 2.5 pl 10×PCR reaction buffer, 0.5 μl 10 mm dNTP mix, 0.5 25 mm MgCl₂, 1 μl cDNA, 1 μl Taq DNA polymerase and 50 pmole of each primer. Adjust volume to 25 μl with distilled water. PCR amplification was performed for 35 cycles (44° C. 1 min, 52° C. 2 min, 72° C. 2 min), with a final elongation step of 10 min at 72° C. Using a robocycler PCR apparatus. S1 gene was amplified by using a 1760-base pair primer. All the isolates yielded a 1760-base pair fragment with 1760-base pair primer. A Lambda DNA/HindIII marker was used to size the clone fragments. This result confirmed all isolates were IBV. The isolated were designated V1, V2, V3, V4 and V5 respectively.

### Cloning and Sequencing S-1 Gene of the 5 Isolates

The S-1 gene PCR products were purified by using a Geneclean® II kit (QBIOgene), and cloned into the plasmid pGEM-T easy vector (Promega) according to the manual. Plasmid PCR and EcoRI digestion was used to confirm the right clone (pT-S). A Lambda DNA/HindIII marker was used to size the clone fragments. The clone was sequenced by primer walking strategy. The primer and its position was listed in Table 33 and the resulting sequence of the isolates were compared by MacDNAsis (Hitachi Software Engineering Co., South San Francisco, Calif.) and PAUP (Sinauer Associates, Sunderland, Mass.).

TABLE 33
Primers used for PCR and sequencing

| | | | PCR | |
|---|---|---|---|---|
| Primer | Sequence | Strain | Product | Position |
| Primer-1 | 5′ CCGAATTCGCTATGAAAACTGAACAAAA | + | 1760 bp | −124 |
| | 3′ (SEQ ID NO: 29) | | | |
| Primer-2 | 5′ GGGTCGACATCCATAACTAACACAAGGG | − | | 1636 |
| | 3′ (SEQ ID NO: 30) | | | |
| IBV-F | 5′ TCAAAGCTTCANGGNGCNTA 3′ (SEQ ID NO: 31) | + | 573-601 | 127 |
| IBV-R | 5′ CTCGAATTCCNGTRTAYTGRCA 3′ (SEQ ID | − | bp | 708 |
| | NO: 32) | | | |
| IBV-FOR | 5′ GTATTCTGCTTTAAAAAG 3′ (SEQ ID NO: 33) | + | 788-800 | 395 |
| IBV-REV | 5′ AGCTCACCACTATAAACA 3′ (SEQ ID NO: 34) | − | bp | 1183 |

The whole sequence of Si gene was obtained by sequencing with three primer pairs (primer-1 and primer-2; IBV-F and IBV-R; IBV-FOR and IBV-REV). In order to get the whole sequence of Si gene, a 800-base pair fragment of S-1 gene for the V1, V2, V3, V4 and V5 isolates was RT-PCR amplified; and three positive clones subcloned into pGEM-T easy vector.

Nucleotide sequences in the N-terminal region of the S-I gene (Table 34) revealed two hypervariable regions and two conserved regions identified by Keeler et al., in the five isolates.

TABLE 34
S-1 gene sequence differences

| | CK4 | | | CK2 |
|---|---|---|---|---|
| | Positions | HV1 | HV2 | Positions |
| | 43 to 47 | Positions 54 to 68 | Positions 116 to 141 | 229 to 236 |
| D41 | HGGAY | .SENNAGTAPSCTAG | .KSGSNSCPLTGLIPKGQIRISAMR- | ..CQYNTG |
| | (SEQ ID | (SEQ ID NO: 36) | SVNSRLHI (SEQ ID NO: 37) | (SEQ ID |
| | NO: 35) | | | NO: 38) |
| V5 | HGGAY | .TISYNAGTA..CTAG | .KAGSN..GLIPKGQIRISAMR-SVNSRLHL | ..CQYNTG |
| | | (SEQ ID NO: 39) | (SEQ ID NO: 40) | |
| Beaudette | HGGAY | .SENNAGTAPSCTAG | .KSGSNSCPLTGLIPKGQIRISAMR- | ..CQYNTG |
| | | (SEQ ID NO: 41) | SVNSRLHR (SEQ ID NO: 42) | |
| V3 | HGGAY | .SENNAG-APSCTAG | .KSGNSCPLTGLIPKGQIRIRMASVNSRLTI | ..CQYNTG |
| | | (SEQ ID NO: 43) | (SEQ ID NO: 44) | |
| Holte | HGGAY | .SENNAGTAPSCTAG | .KAGSNSCPLTGLIPKGQIRISAMR- | ..CQYNTG |
| | | (SEQ ID NO: 45) | SVNSRLHI (SEQ ID NO: 46) | |
| V2 | HGGAY | .SENNAPTQY.SCTAG | .KSGSN..CPLTGLIPKGQIRISAMR- | ..CQYNTG |
| | | (SEQ ID NO: 47) | SVNSRLF (SEQ ID NO: 48) | |
| V4 | HGGAY | .TISY..TAPSCT----AG | ..ISGSNSCPLTGLIP..RISAMR-NVNSRLLI | ..CQYNTG |
| | | (SEQ ID NO: 49) | (SEQ ID NO: 50) | |
| V1 | HGGAY | .SEINAG....... SCTAG | .KAGSNSC..LIPKGQIRISAMR- | ..CQYNTG |
| | | (SEQ ID NO: 51) | SVNSRLHA | |
| | | | (SEQ ID NO: 52) | |

### E. Genotype Analysis

Phylogenetic tree analysis based on the comparison of S-1 nucleotide sequences of 8 IBV strains was conducted. The five IBV field isolates collected from Southern China were highly heterogeneous on the basis of nucleotide sequences (range from 48%-8%). V4 and V5 were categorized as belonging to the same group, and are closer genetically due to a high homology (92%) with the Holte strain than the mass-2 (Beaudette strain). V1, V2, and V3 were classified into different groups. V1 belongs to a new type. V2 and V3 also showed some homology with Holte, but there was also a big variation between them. The D41 was highly homologous with mass-2 (Beaudette strain).

Two hypervariable regions, 56-69 and 117-133, and two conserved regions, 43-47 and 229-236, in the N-terminal amino acid residues of the S-1 gene of Mass-typed IBV had been recognized previously. Both hypervariable regions and conserved regions were identified among the 5 isolates. The results indicate these isolates were mass-typed. However, the phylogenetic tree analysis showed the isolates had some difference with mass-typed strain. Therefore, it is contemplated that the mutation of mass-typed vaccine had led to the emerging field of virulent IBV in Southern China.

The demonstrated high diversity among these isolates indicates that chicken farms at different regions of China may vaccinate their chickens with their respective genotype matched vaccine strains of the particular region. It is contemplated that such genotyping of pathogens (e.g., IBV) may allow genotype specific the production and utilization of the vaccines and methods of the present invention.

### Example 14

### Demonstration of an Immunization of a DNA Vaccine Against Hot Cholera Virus

The example herein demonstrates immunization with a DNA vaccine of with improved efficacy. To demonstrate an improved efficacy of a DNA vaccine against hog cholera virus, a formulated crude DNA vaccine was prepared. After preparation and characterization, the crude bacteria DNA vaccines were administrated to experimental animals and immune responses induced were compared with immunization with naked DNA. A significant improvement in immunogenicity over naked DNA was achieved for both antibody and CTL induction. Specifically, a bacteria DNA vaccine against hog cholera virus induced significantly enhanced serum antibody responses (humoral) and cytotoxic T lymphocyte (cell-mediated) responses in comparison to naked DNA after intramuscular immunization in rabbit.

### A. Plasmid DNA

Immunogenic DNAs pHCV2.5 and pHCV1.25, constructed with the cytomegalovirus promoters that drive expression of the glycoprotein E₂ gene of HCV, have been described herein.

i. Immunization and Vaccine Formulation.

To induce immune responses against HCV, animals were immunized with the following protocol. Female rabbits (1 kg—2 kg) were purchased from the animal center of Hong Kong University. Rabbits were fundamentally immunized with a single intramuscular injection into the right biceps femur muscle with 0.1 ml and 0.5 ml crude bacteria preparations. 0.1 ml formulated vaccine is equal to 100 g naked plasmid DNA and so on. A booster was administered twice at 7 day intervals. The formulated vaccine is prepared as follows:

Inoculate 1 ml bacteria seeds that are propagated from a purified colony to 1.51 LB broth added with 1 mg/ml ampicillin. Incubate at 37° C. and shake at 200 rpm overnight. Spin down to obtain get the bacteria pellet. Weigh and reconstitute to a given concentration, sonication for 10 min, and then add 1 mg/l ampicillin overnight for administration.

ii. Anti-DNA Antibody Responses (ELISA)

An enzyme-linked inumunosorbent assay (ELISA) kit was used to determine antibody responses against HCV according to the manufacturer's manual (IDEXX-Co). The rabbit inoculated with a formulated DNA vaccine had a highest ELISA titer among all the rabbits two weeks later; it is even higher than commercial vaccine. Compared with the NA parameters, the data matched.

iii. Flow Cytometry

Preparation of single cell suspensions (peripheral blood PBLs), whole-blood samples were collected by cardiac puncture into heparinized syringes and placed on ice. The blood was diluted in Han's balanced salt solution (HBSS) without phenol red plus 1.5% fetal bovine serum (Sigma Chemical Co. St. Louis, Mo.) and sieved through a 15 ml rayon wool column primed with HBSS. Five ml of the filtered blood was layered over 5 ml Ficoli Hypaque (Sigma) and centrifuged at 2000 rpm for 10 min. The interface buffy coat was removed with a Pasteur pipette and rinsed three times with 5 ml HBSS. After the final rinse, total lymphocyte from 10⁴ cells per sample was determined by analysis on a flow cytometric apparatus.

Virus neutralization test for HCV CSFV neutralizing antibodies (VNAb) were titrated by the rapid fluorescent focus inhibition test (RFFIT) with modification. PK.15 was used for the detecting of VNAb against CSFV. Anti CSFV VNAb titers are expressed in serum dilution using CSFV C strain as the reference or as the reciprocal serum dilution (rd) that inhibited 50% of the fluorescent focus.

iv. Fever Response in Rabbit after a Challenge Test

Based on the principle that virulent HCV cannot cause fever in rabbits under normal circumstances, while laprinized HCV vaccine can cause fever in the rabbit, it was therefore contemplated that should formulated DNA vaccine induce immune responses in rabbit, it will neutralize the vaccine strain inoculated thereafter, and there will be no fever response.

Five rabbits (2 kg-2.5 kg) were used in this test. Rabbit No. 1 and No. 2 were injected with a high dose and low dose formulated DNA vaccine; No. 3 and No. 4 were injected with naked plasmid DNA and conventional vaccine respectively; No. 5 was set as control.

Firstly, each rabbit was primed with the above materials on day 0. After rabbits were intravenously inoculated with a laprinized vaccine, formulated vaccine and naked plasmid DNA respectively, the last two were bolstered after 2 weeks. Then, each rabbit was bolstered twice at 1 week intervals. Then the rabbits were challenged with the laprinized vaccine strain two weeks later, and the body temperature determined for each rabbit every day for 1 week.

From the temperature curve, the control rabbit had a progressive fever, rabbit No. 3 immunized with the naked plasmid DNA had a fever 4 days later; and the rabbits inoculated with the formulated vaccine (both low dose and high dose) had no fever response. The results indicated that the formulated DNA vaccine had the same efficiency as the HCV laprinized vaccine, while naked plasmid DNA could delay fever by 4 days. Compared with a formulated vaccine, the naked plasmid DNA only had partially protective capabilities.

v. Histopathological Examination of the DNA Vaccine-Injected Muscle

7 days after challenge, muscles of rabbit were resected and fixed with 10% buffered formalin and were embedded in paraffin. Sections were directly used for light microscopic observation. Rabbits injected with naked DNA and conventional commercial vaccines were set as controls.

A histopathological section injected with formulated vaccine showed massive accumulation of mononuclear cells, indicating that strong inflammation was caused by the injection; furthermore, destructive changes of the muscle fibers associated with severe inflammatory cell recruitment were noted, while in the muscle injected with naked DNA and conventional commercial vaccines, the degree of mononuclear cell infiltration and muscle fiber change were distinguishably mild.

vi. Formulated DNA Vaccine in Humoral Immunity (VN)

When rabbits were inoculated with a bacteria preparation of IICV DNA vaccine (pHCV2.5), both the rabbits inoculated with low dose or high dose had a humoral response two weeks later. After booster, the VN titer continued to increase: it still remained at a high level 1 week after challenge. Compared with the naked plasmid DNA and conventional commercial vaccine, the response of neutralization antibody in the rabbit inoculated with the formulated DNA vaccine was even better.

vii. Formulated DNA Vaccine in Cell-Mediated Immune Response

The un-clotted blood of the rabbit was analyzed by using flow cytometry: lymphocytes, monocytes and granular cells of the peripheral blood were counted respectively. The results showed rabbits immunized with the formulated DNA vaccine had the highest concentration of inflammatory cells, especially granular cells. Rabbit No. 2 immunized with a high dose of formulated DNA vaccine had a higher concentration of granular cells; this demonstrates that the non-specific immune response of the rabbit had been greatly enhanced. The rabbit injected with a low dose of formulated DNA vaccine had a relatively low concentration of these cells.

viii Bacterial DNA Vaccine Properties

This example demonstrated that a bacteria formulated DNA vaccine had a better effect over naked DNA vaccines. Superior parameters of Ab titer (ELISA), neutralization activities, cellular immune response and fever response were demonstrated for the bacterial DNA vaccine.

Histopathological examination of inflammatory cells and formulated DNA vaccine injected muscle revealed massive accumulation of inflammatory cells and segmental degeneration and necrosis of myocytes. These characteristics of the DNA bacteria vaccine indicate development strategies for effective DNA vaccine construction, preparation and administration. For example, rabbits immunized with formulated DNA vaccine will cause a cellular immune response. The higher the inoculation dose, the higher immune response it will cause: demonstrating superior properties than the commercial vaccine. It is contemplated that these characteristics may be this is due to the bacteria protein or bacteria sequence. In contrast, the effect caused by naked DNA was not as significant as formulated DNA vaccine. Moreover, it is contemplated that the rabbits had some endurance to the endo-toxin of bacteria, and/or the long term treatment to the bacteria vaccine preparation by ultrasonic techniques produced reduced toxicity, since the bacteria vaccine preparations didn't cause fever or death and clinical symptom to the rabbit. Additionally, there was no gross lesion in the injection site, and the rabbit injected with a low dose had been well absorbed, only the rabbit injected with a high dose had some tyromatosis, indicating it had a strong inflammation response once before.

### Example 15

### Demonstration of a DNA Vaccine against a Regional strain of PRRSV

This example demonstrates a DNA vaccine in the generation of both humoral and cellular immune responses and the ability of a crude bacteria formulation to act as an adjuvant. Specifically, this example demonstrates the immunogenicity of the DNA plasmid constructs in pigs and the use of the ORF 5 polypeptide of PRRSV for specific recognition by T cells to elicit cell-mediated immunity.

An understanding of the humoral immune responses to structural proteins of PRRSV following vaccination is useful for development of an effective vaccine strategy. Furthermore, the production of recombinant vaccines depends on selection of a viral protein that will induce the proper antibody response to protect the animal from disease. The development of humoral immune responses described herein was monitored by ELISA, viral neutralization (VN) and immunoblotting assays (Nelson E. A., et al., 1994, J. Veterinary Diagnostic Investigation 6, 410-415). The role of cell-mediated immunity (CMI) in protection against viral diseases had been widely documented. Most notably, it has been shown that cytotoxic T cells are critical for viral clearance of a number of viruses. The identification of epitopes recognized by T cells has proven to be useful in the design of subunit vaccines for induction of effective immune responses to various microbial pathogens. Previous studies have demonstrated that infected pigs develop CMI responses to PRRSV (Bautista E. M., et al., 1996, Archives of Virology 14, 1357-1365). However, it was unknown whether PRRSV polypeptides differ in their ability to elicit T cell immune responses in swine.

PRRSV ORF 5 envelope protein and ORF 6 matrix protein regions were chosen for the production of recombinant construct as a DNA vaccine. As mentioned (Pirzadeh B. and Dea S., 1997, J General Virology 78, 1867-1873), PRRSV GP has a role in virus infectivity and may function in attachment to cell receptors and/or in virus penetration into the cytoplasm of target cells. It is indicated that at least one neutralizing antigenic determinant is associated with PRRSV GP. Since PRRSV GP is rather abundantly present in the virion and is partially exposed in association with the lipidic envelope (Mardassi H., et al., 1996, Virology 221, 98-112; Meulenberg J. J. M, et al., 1995, Virology 206, 155-163), it shows that GP is the major viral envelope glycoprotein being recognized by most convalescent pig sera (Meulenberg J J. M, et al., 1995, Virology 206, 155-163; Nelson E. A., et al., 1993, J Clinical Microbiology 31, 3184-3189). Then in the case of ORF 6 matrix protein, Bautista (Bautista E. M., et al., 1999, Archives of Virology 144, 117-134) indicates that the-greater T cell proliferation response induced by in vitro stimulation with the product of ORF 6, as compared to the other polypeptides of PRRSV. It indicates that the matrix protein may have a major role in cell mediated immunity to PRRSV. The matrix polypeptide gene is the most conserved gene among all the PRRSV isolates tested (Kapur V., et al., 1996, J General Virology 77, 1271-1276; Meng X. J., et al., 1995, Archives of Virology 140, 745-755; Meng X. J. et al., 1995, J General Virology 76, 3181-3188; Meng X. J., et al., 1996, J General Virology 77, 1265-1270; Meng X. J., et al., 1994, J General Virology 75, 795-801; Murtaugh M. P., et al., 1995, archives of Virology 140, 1451-1460), indicating that the structure of this polypeptide may be essential for the assembly of PRRSV. The matrix protein of PRRSV was then selected for a vaccine of the present invention to demonstrate immunization.

The genomic regions encoding ORFs 5 and 6 of the regional strain PRRSV were selected and cloned into the mammalian expression vector pcDNA3.1(+) to construct a DNA vaccine. DNA immunization with plasmid encoding GPS or ORF 6 of PRRSV, under the control of a human cytomegalovirus promoter, induced anti-GP5 neutralizing antibodies or ORF 6 specific antibodies in both Balb/c mice and its natural host, pigs. The GP5 protein specificity of neutralizing sera was confirmed by immunoblotting, ELISA as well as neutralization assay. This result indicates that neutralizing epitopes for this regional strain PRRSV are present on the viral envelope glycoprotein only.

Inoculation of crude bacteria conjugated with ORF 5-plasmid construct resulted in a stronger antibody level than with plasmid construct alone as shown in the ELISA test. In addition, cellular immune response was detected in immunized mice and pigs by flow cytometrically counting the increase in the number of T cells (CD4+ and CD8+ populations). It was demonstrated that ORF 5 plasmid construct with crude bacteria as an adjuvant may be a used as a vaccine against this regional PRRSV.

### A. Animals

Seven-week-old male Balb/c mice were purchased from the Laboratory Animal Unit of the University of Hong Kong and separated in groups of five mice per cage. Mice were randomly divided into seven experimental groups. Eight piglets weaned at 5 weeks of age were obtained from a breeding farm in the Department of Veterinary Medicine, South China Agricultural University, Guangzhou, 510642. The breeding piglets were tested and proven to be seronegative for PRRSV. The piglets used in this example were randomly divided into four groups.

### B. Virus

The regional strain AV Guangzhou field isolate PRRSV was initially isolated from tissue samples of PRRSV infected pigs in Guangzhou and propagated in MARC-145 cells, a clone of MA-104 cells highly permissive to PRRSV (Kim H. S., et al., 1993, Archives of Virology 133, 477-483). Virus titers were expressed as tissue culture infective dose 50 (TCID50) per ml, as previously described (Dea S., et al., 1992, Canadian Veterinary Journal 33, 801-808).

### C. Recombinant Constructs

Viral RNA was extracted from AV strain PRRSV-infected MARC-145 cells as previously described (Mardassi H., et al., 1995, Archives of Virology 140:1405-1418). Oligonucleotide primers enabling the amplification of PRRSV ORFs 5 and 6 were designed based on the nucleotide sequence of the genome of the American strain VR-2332. HindIII or Xba1 restrictions (RE) sites for ORF 5 region and BamHI or EcoPJ for ORF 6 region were incorporated at the 5′ end of the primers to facilitate cloning. The ORF 5 and ORF 6 regions were first cloned into the TA-cloning vector (pGEM®-T and pGEM®-T Easy Vector Systems, Promega) and then directly inserted into specific RE sites of the pcDNA3.1(+) expression vector (Invitrogen) resulting in the production of pcDNA3.1-ORFs 5 and 6 constructs. Recombinant constructs were verified by sequencing and then transformed into E. Coli strain Top-10. Bacterial culture pellet was dried and sonicated in preparation for injecting the animals.

### D. Transient Expression of the Recombinant Proteins

In vitro expression of the pcDNA3.1(+)-ORF 5 or 6 constructs were tested in transient expression experiments in HEK-293 cells maintained as confluent monolayers. Cells in 6-well tissue culture plates were transfected with 5 pg plasmid DNA by liposome. For immunoflourescent staining, cells were incubated at 37° C. and fixed with 37% formalin for 15 minutes at room temperature at various times (18 hours-72 hours) post-transfection. The monolayers were then reacted for 1 hour with anti-PRRSV polyclonal antibody (National Veterinary Services Laboratories, Ames, Iowa) and the immune reaction was determined following incubation with FITC-conjugated secondary antibody (Zymed).

Transient expression of the cloned ORF 5 and ORF 6 gene were detected by immunofluorescent staining. Expression of the protein product was demonstrated in HEK-293 cells at 72 hours post-transfection. The identification of the protein product was confirmed by using porcine anti-PRRSV serum. Secondary antibody with FITC-conjugated was added to give signals to the transfected cells. Green color fluorescent signals were detected under fluorescence microscopy. The transfected cells showed bright homogenous cytoplasmic reaction by using polyclonal antibody and the expressed proteins tended to accumulate near the perinuclear region. In order to confirm the expressed proteins represented the PRRSV envelope protein and the matrix protein, Western immunoblotting was carried out using the porcine anti-PRRSV serum. Specific protein bands with size 26 kDa and 19 kDa were observed in either lane, which corresponded to envelope and matrix protein respectively.

### E. Mice Immunization Schedule

In vivo expression of pcDNA3.I-ORF 5 or 6 was verified by immunizing groups of five Balb/c mice with 500 μg of either ORF 5 or ORF 6 construct or 100 μg of ORF 5 construct; a mixture of 100 μg of each construct or crude bacteria with 50 μg or 10 μg of ORF 5 construct plasmid equivalent was diluted in 250 μl PBS. DNA was injected into the tibialis cranialis muscle with a 27-gauge needle. The mice were boosted twice with the same quantities of DNA at 2-week intervals. Control mice received 100 μg of pcDNA3. 1 (+) vector via an identical route.

Following DNA inoculation, the protein specificity of mouse sera to GP5 or ORF 6 was established by ELISA and by immunoblotting with expressed proteins. Antibodies against PBRSV antigen in the ELISA were tested positive at various time post-immunizations, indicating a humoral immune response had been generated. In general, antibodies specific to PRRSV were increased in all mice after the first booster of the constructs (day 0). The second booster occurred on day 14. Antibody titer rose to maximum value by 21 days post-immunization (PI) for groups injecting with DNA plasmid constructs and then began to decline. But for groups injecting with crude bacteria with plasmid equivalent, antibody titer rose to maximum value by day 14 PI and remained at a steady high level. Serum neutralization assay was performed using day 35 mice sera. Sera from three of the four mice inoculated with pcDNA3,1-ORF 5 and one of the three mice inoculated with mixture ofpcDNA3,1-ORES and pcDNA3.1-ORF6, together with four of the five mice inoculated with crude bacteria pcDNA3.1-ORF 5 plasmid equivalent demonstrated in vitro neutralizing activity at day 35 of PI. Neutralization was not observed in mice injected with pcDNA3,1-ORF 6, or sera obtained from the vector-injected control mice and pre-immune sera, These results indicate that PRRSV-neutralizing antibodies were specifically targeted to the epitopes of proteins encoded by ORF 5 of PRRSV.

### F. Pig Immunization Schedule

Two piglets in each group were injected three times at 2-week intervals with 100 μg ORF 5 plasmid construct or crude bacteria with 100 μg or 500 μg of ORF 5 plasmid equivalent diluted in 5 ml PBS. PIG 1 and PIG 2 were administered 100 μg ORF 5 plasmid construct; PIG 3 and PIG 4 were administered 100 μg control vector peDNA3.1; PIG 5 and PIG 6 were administered crude bacteria with 100 μg ORF 5 plasmid equivalent; and PIG 7 and PIG 8 were administered crude bacteria with 500 μg ORF 5 plasmid equivalent. Control piglets received 100 μg of pcDNA3.1 (+) vector via an identical route. Two-thirds of the volume was injected, using a 22-gauge needle, into the tibialis cranialis muscle of the leg and one-third was intradermally administered into the dorsal surface of the ear. The first booster was administered day 0, the second booster was administered day 14 and the third booster administered day 28. Preimmune and hyperimmune sera and blood lymphocytes were collected from all mice and pigs prior to each plasmid inoculation to evaluate their immune responses.

Seroconversion was also demonstrated in pigs by ELISA whereas the protein specificity of pig sera to GP5 was established by immunoblotting with samples taken 35 days post-innoculation. All pigs developed antibodies after immunization, but pigs injected with crude bacteria with ORF S plasmid equivalent (PIG 5-8) showed a stronger antibody response than pigs injected with ORF 5 plasmid construct only (PIG 1-2). Each pig may show variation in response towards vaccination, only PIG 6 and 7 developed a faster antibody response after the third booster, but only PIG 7 was able to maintain a high-level antibody level for about two weeks, then started to decline. Neutralizing antibodies were detected in sera of the DNA-immunized pigs only 2 weeks after the second booster injection and in vitro neutralizing activities still maintained until the end of the experiment. Thus, the ability of the DNA vaccine to produce a humoral immune response in another animal model (i.e., pigs) was demonstrated.

### G. Virus Neutralization and Serological Tests

Under some circumstances, as in vaccination, it may be desirable to enhance the normal immune response by administering an adjuvant with the antigen. Adjuvants enhance the body's immune responses to the antigen. A large variety of compounds have been employed as adjuvants. In the type of bacterial fractions, endotoxins enhance antibody formation if given about the same time as the antigen. They have no effect on delayed hypersensitivity, but they can break tolerance, and they have a general stimulatory activity. Endotoxins act by stimulating macrophage production of interleukin-1. Thus the use of crude bacteria as adjuvant shows substantial facilitating effects on the antigen-specific serum and antibody responses. To determine if the anti-ORF 5 and anti-ORF 6 protein monoclonal antibodies could neutralize virus infectivity, virus neutralization assay tests done on MARC-145 cells in the presence of 106.5 TCID50 of the virus.

Following DNA inoculation, humoral immune responses in each animal are monitored by ELISA and immunoblotting prior to the detection of antibody by the virus neutralization assay test. The virus neutralization assay test is less sensitive than the other tests; it could be affected by circulating immune complexes, or neutralizing antibody may actually not appear until later after initial infection. Also, the antigenic epitope(s) associated with virus neutralization may represent only one of many antigenic epitopes on a viral protein. Therefore, the immunoblotting assay detects antibody prior to the appearance of neutralizing antibody.

Mouse and pig sera were tested for the presence of specific anti-GP5 or ORF 6 antibodies by virus neutralization (VN), ELISA and Western immunoblotting tests. The VN tests were performed in duplicate with 100:1 serial dilutions of virus (10⁶⁴ TCID₅₀) in the presence of 50 fold dilution of heat-inactivated test sera, incubated for 60 minutes at 37° C. The mixtures were put in contact with confluent monolayers of MARC-145 cells seeded in 96-well plates 48 hours earlier. Cell monolayers were incubated at 37° C. and observed daily for the appearance of cytopathic effect (CPE). Neutralizing index was expressed by subtracting the reciprocal of log 10 highest virus dilution liter showing no CPE with test serum from log 10 liter with non-immune control serum. Humoral immune responses in each animal were monitored by ELISA using the PRRSV antibody test (IDEXX HERDCHEK PRRS, Westbrook, Me.) and Western blot assays were also performed (Pirzadeh B, and Dea S., 1997, J General Virology 78, 1867-1873) using ORF 5 expressed protein as antigen.

In vivo expression of foreign proteins via simple injection of plasmid DNA with or without crude bacteria conjugate into mice and its natural host was demonstrated. It was found that circulating antibodies to PRRSV are detected 14 days PI after the first dose booster by the ELISA test, whereas antibodies could still be detected by ELISA for the entire sampling period. Interestingly, it was found that those groups of animals injected with crude bacteria as adjuvant showed a higher antibody level and maintained at a steady high level after the second booster rather than declining as in other groups with only plasmid injection. The lower liters of antibodies in DNA-immunized mice and pigs as compared to the crude bacteria-ORF 5 immunized animals could be explained by the fact that the injected antigens are available to the B cells and other antigen-presenting cells which can potentially stimulate a strong antibody response.

These results demonstrate the presence of neutralization epitopes on proteins encoded by ORF 5 but not on ORF 6 encoded proteins. ORF 5 of PRRSV is has been described to elicit neutralizing antibody in pigs and Balb/c mice (Pirzadeh B. and Dea S., 1998, J General Virology 79, 989-999). It is important to note that neutralizing antibodies can only be detected with the association of the conformational neutralizing epitopes on PRRSV protein. DNA constructs poDNA3.I-ORFS and ORF6 have an advantage in that expression in mammalian cells mimics proper viral protein conformations. These constructs thus have the ability to drive antigen production in host animals resulting in immune responses that recognize native PRRSV antigens in a relevant and protective fashion (Kwang J., et al., 1999, Research in Veterinary Science 67, 199-201).

### H. Flow Cytometric Analysis of Peripheral Blood Lymphocytes (PBL)

Peripheral blood lymphocytes were prepared from heparinized blood. 0.2 ml of fresh whole blood was added into 5 ml of freshly prepared red blood cell lysing buffer (0.8% ammonium chloride, 0.083% sodium hydrogencarbonate and 0.003% EDTA-fee acid, pH 7.3) using PYREX Borosilicate glass disposable culture tube (CORNING, NEW YORK) until complete lysis of red blood cells took place. Lymphocytes were collected by centrifugation at 2000 rpm for 2 minutes. Lymphocytes were washed with 1 ml of 1× phosphate buffered saline (PBS) by vortexing the tube, and spun down at 2000 rpm for 2 minutes. Lymphocytes were then stained with appropriate FITC conjugated CD4 or R-PE conjugated CD8a (BD PharMingen) at the recommended concentration (1 μg per 1×106 cells) in darkness for 1 hour with constant shaking. Before running the analysis in a flow cytometer (EPICS ELITE ESP COULTER), 0.5 ml of wash buffer (0.1% sodium azide, 1% Bovine Serum Albumin BSA in 1× phosphate buffered saline PBS) was added into each tube to lop up the volume. At least 5000 cells were analyzed for each sample.

The ability of the DNA vaccine constructs to induce a cell-mediated immune (CMI) response was determined by estimating the increase in CD4+ and CD8+ T cell populations at various times post-immunization in mice. Boosters for tests involving measurement of CD4+ were given on days 0 and 14. A negligible amount of lymphoproliferation took place in response to mice immunized with control DNA plasmid. 83% of the immunized mice demonstrated cell mediated immune responses. Most prominent was the change in the percentages of CD4+ T cells, which peaked on day 14 and 25 of PI at roughly 50% and 42% respectively above the level of day 0. Whereas in the percentages of CD8+ T cells, which peaked on day 14 and 28 of PI at roughly 52% and 37.5% respectively above the level of day 0.

On day 14 of PI, mice of group 5 inoculated with a mixture of both constructs induced the strongest increase in percentages of both T cells. High percentages of T cells also showed in mice of group 4 inoculated with ORF 6 construct. These results indicated the ability of ORF 6 protein to induce higher T cell proliferation response.

In pigs, boosters for tests involving measurement of CD4+ were given on days 0, 14 and 28. Peripheral T cell populations of growing pigs showed then-numbers to drop transiently at day 14 post-immunization (PI) except CD8+ T cell populations in PIG 1. Further analysis on the overall trend of the T cell populations found an increase in CD4+ T cell populations, with a decrease in CD8+ T cell populations or vice versa. This observation was shown most significantly in PIG I and PIG 2. The decline in CD8+ T cell populations (a marker for cytotoxic T cells, which recognize virus-infected cells) continued for at least 3 weeks, whilst CD4+ cells (which include T-helper cells, involved in immunological memory) increased, peaking in range from days 28-42 in different pigs. This result may confirm the ability of the PRRSV to suppress the immune system of its host as this result was not shown in mice.

Another characteristic of porcine T cells included a reversal of the CD4/GD8 ratio of T cell subsets, i.e., the population of CD8+ T cells is higher than CD4+ T cells in porcine subjects, which was reversed in humans and mice. Moreover, the porcine T cell population was unique in that there was a large percentage of CD4+/CD8+ dual expressing peripheral T cells, the percent of this dual expressing cells also increased with the age of the pigs. The result was evaluated in two-color flow cytometric analysis.

It was demonstrated that during DNA constructs immunization in mice a strong influx of both CD4+ T cells and CD8+ cytotoxic T lymphocytes in the peripheral blood. This finding strongly indicates that the presence of cytolytic cells in the peripheral blood during primary immunization is protective. Cytotoxic T cells are potent at lysis of infected cells and may thus prevent spread of the virus (Samsom J-N., et al., 2000, J General Virology 497-505).

Furthermore, this type of cells has been shown to regulate cellular immunity via the production of interferon-γ whereas CD4+ T cells are implicated in eliciting an antibody response.

On the contrary, peripheral T cell populations of growing pigs show their numbers to drop transiently at 14 days post-immunization. During the sampling period, it shows an increase in CD4+ cells and a decrease in CD8+ cells in young pigs or vice versa. An increased level of proliferation response after secondary exposure was due to CD4+ cells that were effectors in this response. Many theories have been offered proposing a mechanism whereby PRRSV may alter the population of T cell subsets. Parallels with other virus infections give rise to the possibility of CD4+ T cell death, perhaps with concurrent CD8+ stimulation or that the virus may act at the level of T cell intra-thymic differentiation (Drew T. W., 2000, Veterinary Research 31, 27-39). Shimizu (Shimizu M., et al., 1996, Veterinary Immunological Immunopathology 50, 19-27) proposed that the cause of CD8+ cell proliferation is a consequence of PRRSV infection. Based on the findings that PRRSV ORF 5 region has an apoptotic effect on transfected cells (Suarez P., et al., (1996). J Virology 70, 2876-2882).

PRRSV may cause immunosuppression in its host. It was uncertain whether the decrease in CD8+ cytotoxic T cells correlated with the apoptotic ability of ORF 5 region of PRRSV. Apoptosis is a critical process in the normal functioning of the immune system. Thus the destructive cytotoxic effects of CD8+ T cells on target cells are all mediated by apoptosis. The difference of response in T cell population after immunization in mice and pigs showed that there was a variation in pathogenicity of the homologous virus among different hosts.

Pigs, like other species, have the typical CD4−/CD8+ and CD4+/CD8− T lymphocytes in their peripheral blood and secondary lymphoid organs. These cells are shown to have helper and cytolytic functions. However, unlike humans and mice, swine also have a prominent CD4/CD8 double positive (DP) lymphocyte population, comprising between 8% and 64% of the circulating pool of small resting T lymphocytes (Zuckennann F. A, 1999, Veterinary Immunology and Immunopathology 72, 55-66). The relative proportion and the absolute number of this lymphocyte subset in the peripheral blood of swine increases gradually with age. This cell population represents an independent cell lineage with no direct relation to mature CD4+ or CD8+ single positive T cells. However, dual DP cells could represent memory CD4+ T helper lymphocytes that have acquired the CD8 antigen upon prior sensitization and retained it after reversion to small lymphocytes. These cells are capable of helping B cells to produce antibody. And because of the predominance of DP cells in older pigs, it is likely that these play a major role in protective immunity due to the co-expression of CD4 and CD8 which might be advantageous for recognition of nominal antigen.

This example shows that DNA immunization with a plasmid encoding GP5 of PRRSV is able to develop both the humoral and cell-mediated immunity in mice and its natural host. Therefore, it appears that the ORF 5 may be a good candidate for a recombinant type vaccine against this regional PRRSV. However, the genomic variability of the ORF 5 genes which was recently reported between North American and European strains (Mardassi H., et al., 1995, Archives of Virology 140, 1405-1418), indicates that these variabilities may be evaluated in terms of the significance of the antigenic determinants involved in protection for the production of a vaccine of the present invention.

DNA immunization is not sufficient to inhibit virus persistence and shedding in the respiratory tract after virus challenge (Pirzadeh B. and Dea S., 1998, J General Virology 79, 989-999). And it was found that mucosal immunity is believed to play a role in protection against PRRSV infection, virus persistence and shedding; thus this aspect of immunity against PRRSV was evaluated. Accordingly, efforts to develop a multivalent DNA vaccine for PRRS and associated serological tests under another PRRSV antigen, ORF 5, was attempted, however, a preliminary humoral immune response obtained. Recently, an infectious clone was generated (Meulenberg J. J, M., et al., 1998, J. Virology. 72, 380-387) and it is contemplated that this could be mutagenised at a specific site to introduce a marker or to reduce the virulence and develop a safe and effective marker vaccine for this virus.

### Example 16

### Demonstration of a Bivalent Vaccine Against Two Pathogens

The example herein describes construction of bivalent vaccine with efficacy against two pathogens. As would be understood by one of ordinary skill in the art, bi- or multivalent vaccines of the present invention may be created using modem molecular cloning technology in light of the disclosures herein:

Laryngotracheitis Virus (ILTV) is a DNA herpes virus that causes a severe upper respiratory disease in chicken. Infectious Bronchitis Virus (IBV) is a member of the coronavirus, a single-stranded RNA virus. They are highly species-specific and both viruses cause significant economic loss in the poultry industry if not under control. In one embodiment of the invention, a bivalent virus was created to both diseases. A recombinant vaccine was created by deleting the TK gene from the ILTV genome and inserting the SI gene subcloned from the 1BV genome. Recombinant virus was generated by transforming chicken embryo kidney cells with the recombinant ILTV-IBV construct. The recombinant virus was shown to induce immunity against both ITLV and IBV in chickens challenged with ITLV and IBV. It is possible to administer the vaccine in water and/or mist.

### REFERENCES

1. U.S. Pat. No. 5,914,123 (Arntzen, Lam).
2. U.S. Pat. No. 6,136,320 (Arntzen, Lam/Prodigene).
3. U.S. Pat. No. 6,194,560 (Arntzen, Mason, Haq/TAMU).
4. Azad, A. A. et al., 1985, Virol. 143: 35-44.
5. Azad A. A. at al., 1987, Virol. 161: 145-152.
6. Bahey-El-Din, M., Casey, P. G., Griffin, B. T. & Gahan, C. G. (2008) Vaccine 26, 5304-5318.
7. Bautista E. M., et al., 1996, Archives of Virology 14, 1357-1365.
8. Bautista E. M., et al., 1999, Archives of Virology 144, 117-134.
9. Bayliss et al., 1990, J. Gen Virol. 71, 1303-1312.
10. Benfield, D. A., et al., 1992, Diseases of Swine 7th Ed, Ames, Iowa: Iowa State University Press: 756-762.
11. Benfield, D. A., et al., 1992, J Vet Diagn Invest. 4: 127-133.
12. Bevan, M. W. et al., Int. Rev. Genet. 16, 357 (1982).
13. Birghan et al., 2000, EMBO Journal 4: 114-123.
14. Bresson J L, Perronne C, Launay O, et al. Safety and immunogenicity of an inactivated split-virion influenza ANietnam/1194/2004 (H5N1) vaccine: phase I randomised trial. Lancet 2006; 367:1657-64.
15. Cavanagh, D. 1997, Arch of Virol. 142: 629-633.
16. Chen Z, Yoshikawa T, Kadowaki S, Hagiwara Y, Matsuo K, Asanuma H, et al. Protection and antibody responses in different strains of mouse immunized with plasmid DNAs encoding influenza virus haemagglutinin, neuraminidase and nucleoprotein. J Gen Virol 1999; 80(Pt 10):2559-64.
17. Chen, M. W., Cheng, T. J. R., Huang, Y., Jan, J. K., Ma, S. H., Yu, A. L., Wong, C. H. & Ho, D. D. (2008) Proc. Natl. Acad. Sci. USA 105, 7235-7240.
18. Chen et al., Jan. 13, 2009, U.S. Pat. No. 7,476,686, Substituted Aryl Thioureas and Related Compounds; Inhibitors of Viral Replication.
19. Cho, H. J., Shin, H. J., Han, I. K., Jung, W. W., Kim, Y. B., Sul, D. & Oh, Y. K. (2007) Vaccine 17, 607-616.
20. Collett, M. S., 1992, Comparative Immunology, Microbiology and Infectious Diseases 15: 145-154.
21. Conzelmann, K. K. et al., 1993, Virol 193: 329-339.
22. Curtiss and lhnen, WO 90/0248, published Mar. 22, 1990.
23. David H. Holman, Danher Wang, Nicholas U. Raja, Min Luo, Kevin M. Moore, Jan Woraratanadharm, Nutan Mytle, John Y. Donga, Multi-antigen vaccines based on complex adenovirus vectors induce protective immune responses against H5N1 avian influenza viruses. Vaccine (2008) 26, 2627-2639.
24. Davis S S. The use of soluble polymers and polymer microparticles to provide improved vaccine responses after parenteral and mucosal delivery. Vaccine 2006; 24(Suppl. 2):S7-10.
25. Dea S., et al., 1992, Canadian Veterinary Journal 33, 801-808.
26. Dea et al., Ultrastructural characteristics and morphogenesis of Porcine Reproductive and Respiratory Syndrome Vrus propagated in the highly permissive Marc-145 cell clone. Plenum Press, New York 1995.
27. Dial, D., et al., 1990, MSD Corn Mtg. Denver: Livestock Conservation Institute, 3.6.
28. De Jong, M. F. et al., 1991, European Comm Seminar on the New Pig Disease, 4:29-30 #4.
29. Denecke, J. et al., EMBO-J 11, 2345 (1992).
30. de Ruyter, P. G., Kuipers, O. P. & de Vos, W. M. (1996) Appl. Environ. Microbiol. 62, 3662-3667.
31. Drew T. W., 2000, Veterinary Research 31, 27-39.
32. Eichelberger, M., Golding, H., Hess, M., Weir, J., Subbarao, K., Luke, C. J., Friede, M.& Wood, D. (2007) Vaccine 26, 4299-4303.
33. Epstein S L, Kong W P, Misplon J A, Lo C Y, Tumpey T M, Xu L, et al. Protection against multiple influenza A subtypes by vaccination with highly conserved nucleoprotein.
Vaccine 2005; 23(46-47):5404-10 [Epub 2005 Jun. 13].
34. Eterradossi, N. et al., 1995; Paris OIE.
35. Feltquate, D. M., et al., 1997, J. Immunol, 158:2278-2284.
36. Francki, R. I. B. et al., 1991, Arch of Virol Supp 2:223-233.
37. Fritsch, Maniatis and Sambrook., 1989, Molecular Cloning, A Laboratory Manual (Second Edition. pp 18.47-18.75).
38. Fuji, Toshio, Jun. 30, 2009, U.S. Pat. No. 7,553,956, Polynucleotide Probe and Primer for Detecting Beer-Clouding Lactic Acid Bacteria and Method of Detecting Beer-Clouding Lactic Acid Bacteria.
39. Gallichan W S, Rosenthal K L. 1995, Vaccine. 13:1589-1595.
40. Gao W, Soloff A C, Lu X, Montecalvo A, Nguyen D C, Matsuoka Y, et al. Protection of mice and poultry from lethal H5N1 avian influenza virus through adenovirus-based immunization. J. Virol. 2006; 80(4):1959-64.
41. Ge, Q., McManus, M., Nguyen, T., Shen, C.-H., Sharp, P. A., Eisen, H. N., Chen, J. RNA interference of influenza virus production by directly targeting mRNA for degradation and indirectly inhibiting all viralRNA transcription. Proc. Natl. Acad. Sci. 2003; 100: 2718-2723.
42. Gilbert C, Robinson K, Le Page R W F, Wells J M. Heterologous expression of an immunogenic pneumococcal type 3 capsular polysaccharide in Lactococcus lactis. Infect Immun 2000; 68:3251-3260.
43. Gilbert et al., 1997, J. Clinical Mcrobiology January 35(1), 264-267.
44. Goddard, et al., 1994 Pet Rec. September 17; 135(12):273-274.
45. Grabstein, K. H., et al., 1994, Science, 264:961-965.
46. Grangette, C., Muller-Alouf, H., Geoffroy, M. C., Goudercourt, D. & Turneer, M. (2002) Vaccine 20, 3304-3309.
47. Gruzza, M., Fons, M., Ouriet, M. F., Duval-Iflah, Y. & Ducluzeau, R., (1994) Microb. Releases 2, 183-189.
48. Harrop et al., Jun. 2, 2009, U.S. Pat. No. 7,541,044, Administration of 5T4 Antigen and Immune Response of Cells Expression 5T4 and CEA Antigens.
49. Hata, T. and T. Maruoka, Dec. 25, 2001, U.S. Pat. No. 6,333,188, Lactic Acid Bacteria Preparation Having Biopurification Activity.
50. S. M. Haugejorden, S. M. et al., J Biol. Chem. 266, 6015 (1991).
51. Hee-Jeong Cho, Hye-Jeong Shin, In-Kwon Han, et al. Induction of mucosal and systemic immune responses following oral immunization of mice with Lactococcus lactis expressing human papillomavirus type 16 L1. Vaccine, 2007; 17: 607-616.
52. Herman, E. M. et al., Planta 182, 305 (1991).
53. Hirt H, Erlandsen S L, Dunny G M. Heterologous inducible expression of Enterococcus faecalis pCF₁₀ aggregation substance Asc10 in Lactococcus lactis and Streptococcus gordonii contributes to cell hydrophobicity and adhesion to fibrin. J. Bacteriol. 2000; 182:2299-2306.
54. Hoelscher M A, Garg S, Bangari D S, Belser J A, Lu X, et al. Development of adenoviral-vector-based pandemic influenza vaccine against antigenically distinct human H5N1 strains in mice. Lancet. 2006; 367:475-481.
55. Hofmann, M. A. et al., 1994, Arch Virol 139: 217-229.
56. Horzinek, M., 1991, Arch of Virol Supp 3:1-5.
57. Hudson, P. J., et al, 1986, Nucleic Acids Res 14: 5001-5012.
58. Itoh, Kyogo, Oct. 7, 2008, U.S. Pat. No. 7,432,354, Tumor Antigen.
59. Jeffery, K. & Taubenberger, J. K. (1998) Proc. Natl. Acad. Sci. USA 95, 9713-9715.
60. Johansson, B. E., Brett, I. C., Focosi, D. & Poland, G. A. (2006) N. Engl. J. Med. 354, 2724-2725.
61. Kapur V., et al., 1996, J General Virology 77, 1271-1276.
62. Katz J M, Lu X, Young S A, et al. Adjuvant activity of the heat-labile enterotoxin from enterotoxigenic Escherichia coli for oral administration of inactivated influenza virus vaccine. J Infect Dis 1997;175: 352-63.
63. Katz, J. M., Lu, X., Young, S. A.& Galphin, J. C., 1997, J. Infect. Dis. 175, 352-363.
64. Ke-Qin, Yuka Hoshino, Yoshihiko Toda, et al Immunogenicity and protective efficacy of orally administered recombinant Lactococcus lactis expressing surface-bound HIV Env. Blood, 2003.
65. Kibenge F S, et al., 1997, Arch Virol. 142:2401-2419.
66. Kibenge F S, Dhama V. 1997. Arch. Virol. 142:1227-1236.
67. Kilbourne, E. D., Smith, C., Brett, I., Pokorny, B. A., Johansson, B. & Cox, N. (2002) Proc. Natl. Acad. Sci. USA 99, 10748-52.
68. Kim H. S., et al., 1993, Archives of Virology 133, 477-483.
69. Kimoto, H., Nomura, M., Kobayashi, M., Mizumachi, K.& Okamoto, T. (2003) Can. J. Microbiol. 49, 707-711.
70. Klijn, N., Weerkamp, A. H. & de Vos, W. M. (1995) Appl. Environ. Microbiol. 61, 2771-2774.
71. Kodihalli S, Goto H, Kobasa D L, Krauss S, Kawaoka Y, Webster R G. DNA vaccine encoding hemagglutinin provides protectivebimmunity against H5N1 influenza virus infection in mice. J Virol 1999; 73(3):2094-8.
72. Kong, W. P., Hood, C., Yang, Z. Y., Wei, C. J., Xu, L., Garcia-Sastre, A., Tumpey, T. M. & Nabel, G. J. (2006) Proc. Natl. Acad. Sci. USA 103, 15987-15991.
73. Konig, M et al., 1995, J Virol 69:5479-86
74. Kwang J., et al., 1999, Research in Veterinary Science 67, 199-201.
75. Peggy A. Lalor, Richard J. Webby, Jane Morrow, Denis Rusalov, David C. Kaslow, Alain Rolland, and Larry R. Smith. Plasmid DNA-Based Vaccines Protect Mice and Ferrets against Lethal Challenge with A/Vietnam/1203/04 (H5N1) Influenza Virus. The Journal of Infectious Diseases 2008; 197:1643-1652.
76. U.S. Pat. No. 5,484,719 (Lam, Arntzen).
77. U.S. Pat. No. 5,612,487 (Lam, Arntzen).
78. U.S. Pat. No. 6,034,298 (Lam, Arntzen, Mason).
79. Lam, D. M. K. & Shi, J. (1996) Agro-Food-Industry high-tech March/April, 7-12.
80. Lam, D. M. K., Shi, J. & Zhang, X. Y. (1997) Foreign Medical Sciences China 20, 145-149.
81. Lavelle E C. Lectins and microparticles for enhanced oral vaccination. Methods 2006; 38:84-9.
82. Lin J, Zhang J, Dong X, et al. Safety and immunogenicity of an inactivated adjuvanted whole-virion influenza A (H5N1) vaccine: a phase I randomised controlled trial. Lancet 2006; 368:991-7.
83. Lowings, J. P. et al., 1994, J. Gen Virol 75: 3461-3468.
84. Lowings, P. et al., 1996, J Gen Virol 77:1311-1321.
85. Lu X H, Tumpey T M, Morken T, et al. Amouse model for the evaluation of pathogenesis and immunity to influenza A (H5N1) viruses isolated from humans. J Virol 1999; 73:5903-11.
86. Mardassi et al., 1994. J Clinical Microbiology. September 2197-2203.
87. Mardassi H., et al., 1995, Archives of Virology 140:1405-1418.
88. Mardassi, H. et al., 1996, Virol 221:98-112.
89. Mardassi et al., Can J Vet Res 58, 55-64.
90. Mason, H. S. et al., Proc. Nat. Acad. Sci. USA, 89:11745749 (1992).
91. Meng X. J., et al., 1994, J General Virology 75, 795-801.
92. Meng X. J., et al., 1995, Archives of Virology 140, 745-755.
93. Meng X. J. et al., 1995, J General Virology 76, 3181-3188.
94. Meng X. J., et al., 1996, J General Virology 77, 1265-1270.
95. Mercenier, A., Pavan, S. & Pot, B., 2003, Curr. Pharm. Des. 9:175-191.
96. Meulenberg et al., 1993, Virology 192, 62-74.
97. Meulenberg, J. J, at al., 1993, Virol 192: 62-72.
98. Meulenberg, et al., 1995, Virology 206:155-163.
99. Meulenberg J. J, M., et al., 1998, J. Virology. 72, 380-387.
100. Meyers et al., U.S. Pat. No. 5,811,103, issued Sep. 11, 1998.
101. Meyers et al., U.S. Pat. No. 5,925,360, issued Jul. 20, 1999.
102. Meyers et al., U.S. Pat. No. 5,935,582, issued Aug. 10, 1999.
103. Mulenberg, J. J., et al., 1996, Virol 225: 44-51.
104. Muller H, Nitschke R. 1987, Virology 159:174-177.
105. Mundt et al., 1997, J. Virol 71:5647-51.
106. Munro, S. and Pelham, H. R. B., Cell 48, 988-997 (1987).
107. Murphy et al., 1992, Vet. Microbiol. 32, 101-115.
108. Murtaugh M. P., et al., 1995, archives of Virology 140, 1451-1460.
109. Nelson E. A., et al., 1993, J Clinical Microbiology 31, 3184-3189.
110. Nelson E. A., et al., 1994, J. Veterinary Diagnostic Investigation 6, 410-415.
111. Nochi, T., Takagi, H., Yuki, Y., Yang, L. J., Masumura, T., Mejima, M., Nakanishi, U.,
Matsumura, A., Uozumi, A., Hiroi, T., Morita, S., Tanaka, K., Takaiwa, F. & Kiyono, H. (2007) Proc. Natl. Acad. Sci. USA 104, 10986-10991.
112. Nunoya T. et al., 1992, Avian Drs. 36:597-609.
113. Negrutiu, R. et al., Plant Mal. Biol. 8, 363 (1987).
114. Oblinger, V., 1992, Pig Dis Info Centre.
115. Pierre et al., 1998, Virol 249: 297-306.
116. Pirzadeh B. and Dea S., 1997, J General Virology 78, 1867-1873.
117. Pirzadeh, B., Gagnon, C, A. and Dea, S., 1998, Can J. Vet. Res. 62, 170-177.
118. Pirzadeh B. and Dea S., 1998, J General Virology 79, 989-999.
119. Poo, H., Pyo, H. M., Lee, T. Y., Yoon, S. W., Lee, J. S., Kim, C. J., Sung, M. H. & Lee, S. H. (2006) Int. J. Caner 119, 1702-1709.
120. Raymakers, J. M. L., 1991, European Comm Seminar on PRRS 11:4-5, Brussels, #16).
121. Read R C, Naylor S C, Potter C W, Bond J, Jabbal-Gill I, Fisher A, et al. Effective nasal influenza vaccine delivery using chitosan. Vaccine 2005; 23:4367-74.
122. Robinson K, Chamberlain L M, Schofield K M, Wells J M, Page R W F. Oral vaccination of mice against tetanus with recombinant Lactococcus lactis. Nat. Biotechnol. 1997; 15:653-123. Rodkey, L. S. et al., Apr. 22, 2003, U.S. Pat. No. 6,551,830, Rh Blood Group Antigen Compositions and Methods of Use.
124. Röhm, C., Horimoto, T., Kawaoka, Y., Suss, J. & Webster, R. G. (1995) Virology 209, 664-670.
125. Rowe, T., Abernathy, R. A., Hu-Primmer, J., Thompson, W. W., Lu, X. H., Lim, W., rn Fukuda, K., Cox, N. J. & Katz, J. M. (1999) J. Clin. Microbiol. 37, 937-943.
126. Rumenapf, T. et al, 1993, J Vrol, 67:3288-3294.
127. Samsom J-N., et al., 2000, J General Virology 497-505.
128. Schmid, R. M., et al., 1994, Gastroenterol, 32:665-670.
129. Shih, A. C. C., Hsiao, T. C., Ho, M. S. & Li, W. H. (2007) Proc. Natl. Acad. Sci. USA 104:6283-6288.
130. Shimizu M., et al., 1996, Veterinary Immunological Immunopathology 50, 19-27.
131. Shin et al., 1997.
132. Shin, S. et al., 1998.
133. Speis et al., 1987, Virus Res 8: 127-140.
134. Stark, I., et al., 1990, Virol 174: 286-289.
135. Steensels M, Van Borm S, Lambrecht B, De Vriese J, Le Gros F X, Bublot M, van den Berg T. Efficacy of an inactivated and a fowlpox-vectored vaccine in Muscovy ducks against an Asian H5N1 highly pathogenic avian influenza viral challeng. Avian Dis. 2007 March; 51(1 Suppl):325-31.
136. Stevens, J., Corper, A. L., Basler, C. F., Taubenberger, J. K., Palese, P. & Wilson, I. A. (2004) Science 303, 1866-1870.
137. Suarez et al., 1994 Arch Virol. 135, 89-99.
138. Suarez P., et al., (1996). J Virology 70, 2876-2882.
139. Subbarao, K., Klimov, A., Katz, J., Regnery, H., Lim, W., Hall, H., Perdue, M., Swayne, D., Bender, C., Huang, J., Hemphill, M., Rowe, T., Shaw, M., Xu, X., Fukuda, K. & Cox, N. (1998) Science 279, 393-396.
140. Suguitan A L Jr, McAuliffe J, Mills K L, Jin H, Duke G, Lu B, Luke C J, Murphy B, Swayne D E, Kemble G, Subbarao K. Live, attenuated influenza A H5N1 candidate vaccines provide broad cross-protection in mice and ferrets. PLoS Med. 2006 September; 3(9):e375.
141. Suzuki, Y., Ito, T., Suzuki, T., Holland J. R. E., Chambers, T. M., Kiso, M., Ishida, H. & Kawaoka, Y. (2000) J. Virol. 74, 11825-11831.
142. Thiel, H-J. et al., 1996, Fundamental Virology, 3rd ed. Raven Press New York, 1059-1079.
143. Thiel, H-J, et al., 1991, J. Virol 65: 4705-4712.
144. Tompkins S M, Zhao Z S, Lo C Y, Misplon J A, Liu T, Ye Z, et al. Matrix protein 2 vaccination and protection against influenza viruses, including subtype H5N1. Emerg Infect Dis 2007; 13(3):426-35.
145. Tones, C. A. T., et al., 1997, J Virol, 158:4529-4532.
146. Toro H, Tang D C, Suarez D L, Sylte M J, Pfeiffer J, Van Kampen K R. Protective avian influenza in ovo vaccination with non-replicating human adenovirus vector. Vaccine 2007; 25(15):2886-91 [Epub 2006 Sep. 25].
147. Treanor J J, Campbell J D, Zangwill K M, Rowe T, Wolff M. Safety and immunogenicity of an inactivated subvirion influenza A (H5N1) vaccine. N Engl J Med 2006; 354:1343-51.
148. Treanor J J, Wilkinson B E, Masseoud F, Hu-Primmer J, Battaglia R, O'Brien D, et al. Safety and immunogenicity of a recombinant hemagglutinin vaccine for H5 influenza in humans. Vaccine 2001; 19(13-14):1732-7.
149. Ulmer, J. B., et al., 1996, Immunol, 89: 59-67.
150. Ulmer, J. B., el at., 1996 Cur Opin Immunol, 8:531-536.
151. Van den Berg T P, 1991, Avian Pathol. 20:133-143.
152. Vilcek, S. et al., 1996, Virus Res 43: 137-147.
153. Vanderhallen, H., et al., 1999, Arch Vii al 144: 1669-1677.
154. Wandelt, C. et al., The Plant Journal 2, 181 (1992).
155. Wei, C. J., Xu, L., Kong, W. P., Shi, W., Canis, K., Stevens, J., Yang, Z. Y., Dell, A., Haslam, S. M., Wilson, I. A. & Nabel, G. J. (2008) J. Virol. 82, 6200-6208.
156. Weiland, E. et al., 1990, J Virol 64: 3563-3569.
157. Weiland, E., at al., 1999, Vet Microbiol 66: 171-186.
158. Wensvoort G., et al., 1991, Veterinary Quarterly 13, 121-130.
159. Wenzler, H. C., et al., (1989) Plant Mol. Biol. 12:41-50.
160. Wessels, et al., Dec. 3, 1996, U.S. Pat. No. 5,580,787, Cloning Vector for Use in Lactic Acid Bacteria.
161. Wiskerchen, M. A., et al., 1991, J Virol 65: 4508-4514.
162. World Health Organization. Cumulative Number of Confirmed Human Cases of Avian Influenza A (H5N1) Reported to WHO.
163. Wyeth P J, Chettle N J. 1990, Vet Rec 126:577-578.
164. Xin, K. Q., Hoshino, Y., Toda, Y., Igimi, S., Kojima, Y., Jounai, N., Ohba, K., Kushiro, A., Kiwaki, M., Hamajima, K., Klinman, D. & Okuda, K. (2003) Blood 102, 223-228.
165. Yamaguchi T, et al., 1996, Virology 223:219-223.
166. Zuckennann F. A, 1999, Veterinary Immunology and Immunopathology 72, 55-66.

What is claimed is:

1. A composition for inducing an immune response to an antigen in a subject, comprising a genetically modified lactic acid bacterium expressing the antigen, wherein the bacterium is formulated with mucoadhesive polymers.

2. The composition of claim 1, wherein the lactic acid bacterium is of the genus Lactococcus.

3. The composition of claim 2, wherein the lactic acid bacterium is of the species Lactococcus lactis.

4. The composition of claim 1, wherein the antigen is a chimeric protein.

5. The composition of claim 1, wherein the antigen is from a pathogenic virus or organism primarily affecting livestock or poultry.

6. The composition of claim 1, wherein the antigen is a hemagglutinin of avian influenza virus H5N1.

7. The composition of claim 6, wherein the hemagglutinin is HA1 hemagglutinin protein.

8. The composition of claim 6, wherein the hemagglutinin protein is expressed on the surface of the bacteria via cell wall anchor.

9. The composition of claim 1, wherein the bacterium is formulated into microcapsules.

10. The composition of claim 9, wherein the microcapsules are acid labile microcapsules.

11. The composition of claim 1, wherein the mucoadhesive polymers are hydrophilic polymers or hydrogels.

12. The composition of claim 11, wherein the hydrophilic polymers are selected from the group consisting of polyvinyl pyrrolidone (PVP), methyl cellulose (MC), sodium carboxy methylcellulose (SCMC), and hydroxypropyl cellulose (HPC).

13. The composition of claim 1, wherein the bacterium is formulated in solid dosage forms which are coated with enteric coatings.

14. A method of inducing an immune response to an antigen in a subject, comprising the step of administering to said subject the composition of claim 1, wherein the subject is a human or a non-human animal.

15. The method of claim 14, wherein the immune response is a humoral immune response, mucosal immune response, cellular immune response, or protective immune response.

16. The method of claim 14, wherein the composition is administered orally or by injection.

17. The method of claim 14, wherein the bacteria express a hemagglutinin of avian influenza virus H5N1.

18. The method of claim 14, wherein the composition is formulated in enteric coated solid dosage forms.

19. The method of claim 14, wherein the composition is administered in conjunction with an adjuvant.

20. The method of claim 19, wherein the adjuvant is Cholera Toxin B, QS-21 saponin adjuvant, or DNA immunostimulatory sequences.
